SEQUENCE LISTING (50 sequences)

SEQ ID NO: 1 - DNA, 1515 bp, Glycine max
atggctgtcgtttcgcgaagtgcgacgacctatacgcgccactacttaatacgacacgagtttgataggaaaacgaaaacctgcgttgccaataatagtttgtgttactctgctaagaaggctcctccaccgcagaggattgttggtggccgtagagtgattgttgctgcgagccctcccaccgaagacgctgtagttgccactgaccctctcacgaagcaggatctcgtcgattatcttgcctccggttgcaagcccaaggataaatggagaataggtactgaacatgagaagtttggttttgagattggaagcttgcgtcctatgaagtatgaccaaatagcagaattgctgaatggcattgctgagaggtttgactgggataaagtaatggaaggtgataaaattattggactcaaacaggggaagcagagcatatcattggagcctggtggtcagtttgaacttagtggagctcctcttgaaaccttgcatcagacttgtgctgaagttaattcccacctttatcaggttaaagctgttgctgaggaaatgggaattggatttttggggattggtttccagccaaagtggggaatcaaagacatacctataatgccaaagggaagatacgacatcatgaggaactacatgcctaaagttggctctcttgggcttgacatgatgttcaggacatgcactgtacaggtcaatctggactttagttctgaagctgacatgatcaggaaatttcgtgcaggccttgctttgcagccgatagcaacggctctttttgcaaattcaccctttaaagagggaaagccaaatggttttgtcagtatgagaagccatatttggactgatactgataaggaccgcacaggcatgctgccttttgtttttgatgactcttttgggtttgagcaatatgttgattatgctcttgatgttcctatgtattttgtctatcggaaaaacagatatatcgactgcactggaaagaccttcagggactttttggctggaagacttccttgtattcctggtgaattaccaactctcaatgattgggaaaatcacttgacaactatatttcctgaggtcaggctgaagaggtatttggagatgagaggtgctgatggagggccttggagaagattgtgtgctttaccagcattttgggtagggttattgtacgatgaactttctctaaaaagtgttttggatatgacagctgattggactccagaagaaagacaaatgttaaggaataaggttcctgtaactggtctgaagacaccattccgagacggtttgctgaagcatgttgctgaagatgttctaaagttggcaaaggatggcttggagagaagaggcttcaaggaatcgggatttttgaatgaggttgccgaggtggttagaacaggtgtcactccagctgagaggcttttggaattgtatcatggaaagtgggagcaatccgtagatcatgtgtttgaggaattgctttattaa

SEQ ID NO: 2 - protein, 504 aa, Glycine max
MAVVSRSATTYTRHYLIRHEFDRKTKTCVANNSLCYSAKKAPPPQRIVGGRRVIVAASPPTEDAVVATDPLTKQDLVDYLASGCKPKDKWRIGTEHEKFGFEIGSLRPMKYDQIAELLNGIAERFDWDKVMEGDKIIGLKQGKQSISLEPGGQFELSGAPLETLHQTCAEVNSHLYQVKAVAEEMGIGFLGIGFQPKWGIKDIPIMPKGRYDIMRNYMPKVGSLGLDMMFRTCTVQVNLDFSSEADMIRKFRAGLALQPIATALFANSPFKEGKPNGFVSMRSHIWTDTDKDRTGMLPFVFDDSFGFEQYVDYALDVPMYFVYRKNRYIDCTGKTFRDFLAGRLPCIPGELPTLNDWENHLTTIFPEVRLKRYLEMRGADGGPWRRLCALPAFWVGLLYDELSLKSVLDMTADWTPEERQMLRNKVPVTGLKTPFRDGLLKHVAEDVLKLAKDGLERRGFKESGFLNEVAEVVRTGVTPAERLLELYHGKWEQSVDHVFEELLY

SEQ ID NO: 3 - DNA, 1350 bp, Glycine max
atggcgagccctcccaccgaagacgctgtagttgccactgaccctctcacgaagcaggatctcgtcgattatcttgcctccggttgcaagcccaaggataaatggagaataggtactgaacatgagaagtttggttttgagattggaagcttgcgtcctatgaagtatgaccaaatagcagaattgctgaatggcattgctgagaggtttgactgggataaagtaatggaaggtgataaaattattggactcaaacaggggaagcagagcatatcattggagcctggtggtcagtttgaacttagtggagctcctcttgaaaccttgcatcagacttgtgctgaagttaattcccacctttatcaggttaaagctgttgctgaggaaatgggaattggatttttggggattggtttccagccaaagtggggaatcaaagacatacctataatgccaaagggaagatacgacatcatgaggaactacatgcctaaagttggctctcttgggcttgacatgatgttcaggacatgcactgtacaggtcaatctggactttagttctgaagctgacatgatcaggaaatttcgtgcaggccttgctttgcagccgatagcaacggctctttttgcaaattcaccctttaaagagggaaagccaaatggttttgtcagtatgagaagccatatttggactgatactgataaggaccgcacaggcatgctgccttttgtttttgatgactcttttgggtttgagcaatatgttgattatgctcttgatgttcctatgtattttgtctatcggaaaaacagatatatcgactgcactggaaagaccttcagggactttttggctggaagacttccttgtattcctggtgaattaccaactctcaatgattgggaaaatcacttgacaactatatttcctgaggtcaggctgaagaggtatttggagatgagaggtgctgatggagggccttggagaagattgtgtgctttaccagcattttgggtagggttattgtacgatgaactttctctaaaaagtgttttggatatgacagctgattggactccagaagaaagacaaatgttaaggaataaggttcctgtaactggtctgaagacaccattccgagacggtttgctgaagcatgttgctgaagatgttctaaagttggcaaaggatggcttggagagaagaggcttcaaggaatcgggatttttgaatgaggttgccgaggtggttagaacaggtgtcactccagctgagaggcttttggaattgtatcatggaaagtgggagcaatccgtagatcatgtgtttgaggaattgctttattaa

SEQ ID NO: 4 - protein, 449 aa, Glycine max
MASPPTEDAVVATDPLTKQDLVDYLASGCKPKDKWRIGTEHEKFGFEIGSLRPMKYDQIAELLNGIAERFDWDKVMEGDKIIGLKQGKQSISLEPGGQFELSGAPLETLHQTCAEVNSHLYQVKAVAEEMGIGFLGIGFQPKWGIKDIPIMPKGRYDIMRNYMPKVGSLGLDMMFRTCTVQVNLDFSSEADMIRKFRAGLALQPIATALFANSPFKEGKPNGFVSMRSHIWTDTDKDRTGMLPFVFDDSFGFEQYVDYALDVPMYFVYRKNRYIDCTGKTFRDFLAGRLPCIPGELPTLNDWENHLTTIFPEVRLKRYLEMRGADGGPWRRLCALPAFWVGLLYDELSLKSVLDMTADWTPEERQMLRNKVPVTGLKTPFRDGLLKHVAEDVLKLAKDGLERRGFKESGFLNEVAEVVRTGVTPAERLLELYHGKWEQSVDHVFEELLY

SEQ ID NO: 5 - DNA, 960 bp, Glycine max
atgggaattggatttttggggattggtttccagccaaagtggggaatcaaagacatacctataatgccaaagggaagatacgacattatgaggaattacatgcctaaagttggctctcttgggcttgacatgatgttcaggacatgcactgtacaggtcaatctggactttagttctgaagctgacatgatcaggaaatttcgtgcaggtcttgctttgcagccaatagcaacggctctttttgcaaattcaccctttaaagagggaaagccaaatggttttttcagtatgagaagccatatttggactgatactgacaaggatcgcacaggcatgctgccttttgtttttgatgactcttttgggtttcagcagtatgttgattatgcacttgatgttcctatgtattttgtctatcggaaacacagatatatcgactgtactggaaagaccttcagggacttcttggctggaagacttccttgtattcctggtgaattaccaactctcaatgattgggaaaatcacttgacaactatatttcctgaggtcaggctgaagagatatttggagatgagaggtgctgatggagggccttggagaaggttatgtgctttaccagcattttgggtagggttattgtacgatgaagtttctctacaaagtgttttggatatgacagctgattggactccagaagaaagacaaatgctaaggaataaggttcctgtaactggtttgaagacaccattccgagacggtttgctgaagcatgttgctgaagatgttctaaagttggcaaaggatggcttggaaagaagaggcttcaaggaatcaggatttttgaatgaggttgccgaggtggttagaacaggtgtcactccagccgagaggcttttggaattgtatcatggaaagtgggagcaatccgtagatcacgtgtatgaggaattgctgtat

SEQ ID NO: 6 - protein, 320 aa, Glycine max
MGIGFLGIGFQPKWGIKDIPIMPKGRYDIMRNYMPKVGSLGLDMMFRTCTVQVNLDFSSEADMIRKFRAGLALQPIATALFANSPFKEGKPNGFFSMRSHIWTDTDKDRTGMLPFVFDDSFGFQQYVDYALDVPMYFVYRKHRYIDCTGKTFRDFLAGRLPCIPGELPTLNDWENHLTTIFPEVRLKRYLEMRGADGGPWRRLCALPAFWVGLLYDEVSLQSVLDMTADWTPEERQMLRNKVPVTGLKTPFRDGLLKHVAEDVLKLAKDGLERRGFKESGFLNEVAEVVRTGVTPAERLLELYHGKWEQSVDHVYEELLY

SEQ ID NO: 7 - DNA, 1512 bp, Zea mays
atggcggtggcgtcgcggctggcggtcgcgcgggtgtcgccggacggcgcgcgccccgcggcggcggcggcggcaggggggagggggaggagcgggctcgcggcggttcggctcccgtcgaccgccggttgggtgaggaggagggggcgcggcggggccgtcgcggccagccctcccacggaggaggccgtgcagatgacggagccgctcaccaaggaggacctcgtcgcctacctcgtctccgggtgcaagcccaaggagaattggagaattgggacggagcacgaaaagttcggtttcgaagtcgacactttacgccctttaaaatatgatcagattcgtgacatactgaacggtcttgctgagagatttgattgggacaagataatggaaaaaaacaatgttatcggtctcaagcagggaaagcaaagcatctcactagaacctggaggccaatttgaacttagtggcgctcctctcgaaacattacatcaaacttgtgccgaggtcaattcgcatctttatcaggtcaaggcagttggagaggaaatgggaataggatttcttgggcttggctttcagccaaaatgggcactgagtgacataccaataatgccaaagggaagatacgaaataatgaggaattacatgcctaaagttggtactcttggccttgatatgatgttccggacatgtactgtgcaggttaatcttgacttcagttcagaacaggatatgataaggaaatttcgtgctggcctcgctttgcagcctattgcaactgcaatatttgccaattctccgttcaaagaaggaaaaccaaatggatttctcagcttaaggagccatatctggacagatactgataataatcgtgcagggatgctcccttttgtctttgacgactcatttgggtttgagcaatatgtggactatgcattagaagtccccatgtattttgtgtaccgaaataaaaagtatattgactgcaccggaatgtcgtttcgggattttatgcaaggaaagcttccacaggctcctggggagttgcctactcttaccgattgggagaaccatctaacaacaatttttccagaggttaggctaaagaggtaccttgagatgagaggtgctgatggtggcccatggaggagattgtgtgcgttgcctgcattttgggttgggctgctgtacgacgaggaatcgttacaaagcattttagacatgacttttgattggacaaaggaggaaagagagatgttaagacggaaggtaccatcgactggtttgaagacgccgtttcgtgatggatatgtaagagatttagctgaggaagttctaaaactggccaagaatggactggaaagaagagggtacaaggaggttggtttccttagagaggtcgacgaagtagtgagaacaggagtgacgcctgcggagaggctgctgagcccgtacgagaccaagtggcaacgcaacgtcgaccatgttttcgagcatttgttatactga

SEQ ID NO: 8 - protein, 503 aa, Zea mays
MAVASRLAVARVSPDGARPAAAAAAGGRGRSGLAAVRLPSTAGWVRRRGRGGAVAASPPTEEAVQMTEPLTKEDLVAYLVSGCKPKENWRIGTEHEKFGFEVDTLRPLKYDQIRDILNGLAERFDWDKIMEKNNVIGLKQGKQSISLEPGGQFELSGAPLETLHQTCAEVNSHLYQVKAVGEEMGIGFLGLGFQPKWALSDIPIMPKGRYEIMRNYMPKVGTLGLDMMFRTCTVQVNLDFSSEQDMIRKFRAGLALQPIATAIFANSPFKEGKPNGFLSLRSHIWTDTDNNRAGMLPFVFDDSFGFEQYVDYALEVPMYFVYRNKKYIDCTGMSFRDFMQGKLPQAPGELPTLTDWENHLTTIFPEVRLKRYLEMRGADGGPWRRLCALPAFWVGLLYDEESLQSILDMTFDWTKEEREMLRRKVPSTGLKTPFRDGYVRDLAEEVLKLAKNGLERRGYKEVGFLREVDEVVRTGVTPAERLLSPYETKWQRNVDHVFEHLLY

SEQ ID NO: 9 - DNA, 1350 bp, Zea mays
atggccagccctcccacggaggaggccgtgcagatgacggagccgctcaccaaggaggacctcgtcgcctacctcgtctccgggtgcaagcccaaggagaattggagaattgggacggagcacgaaaagttcggtttcgaagtcgacactttacgccctttaaaatatgatcagattcgtgacatactgaacggtcttgctgagagatttgattgggacaagataatggaaaaaaacaatgttatcggtctcaagcagggaaagcaaagcatctcactagaacctggaggccaatttgaacttagtggcgctcctctcgaaacattacatcaaacttgtgccgaggtcaattcgcatctttatcaggtcaaggcagttggagaggaaatgggaataggatttcttgggcttggctttcagccaaaatgggcactgagtgacataccaataatgccaaagggaagatacgaaataatgaggaattacatgcctaaagttggtactcttggccttgatatgatgttccggacatgtactgtgcaggttaatcttgacttcagttcagaacaggatatgataaggaaatttcgtgctggcctcgctttgcagcctattgcaactgcaatatttgccaattctccgttcaaagaaggaaaaccaaatggatttctcagcttaaggagccatatctggacagatactgataataatcgtgcagggatgctcccttttgtctttgacgactcatttgggtttgagcaatatgtggactatgcattagaagtccccatgtattttgtgtaccgaaataaaaagtatattgactgcaccggaatgtcgtttcgggattttatgcaaggaaagcttccacaggctcctggggagttgcctactcttaccgattgggagaaccatctaacaacaatttttccagaggttaggctaaagaggtaccttgagatgagaggtgctgatggtggcccatggaggagattgtgtgcgttgcctgcattttgggttgggctgctgtacgacgaggaatcgttacaaagcattttagacatgacttttgattggacaaaggaggaaagagagatgttaagacggaaggtaccatcgactggtttgaagacgccgtttcgtgatggatatgtaagagatttagctgaggaagttctaaaactggccaagaatggactggaaagaagagggtacaaggaggttggtttccttagagaggtcgacgaagtagtgagaacaggagtgacgcctgcggagaggctgctgagcccgtacgagaccaagtggcaacgcaacgtcgaccatgttttcgagcatttgttatactga

SEQ ID NO: 10 - protein, 449 aa, Zea mays
MASPPTEEAVQMTEPLTKEDLVAYLVSGCKPKENWRIGTEHEKFGFEVDTLRPLKYDQIRDILNGLAERFDWDKIMEKNNVIGLKQGKQSISLEPGGQFELSGAPLETLHQTCAEVNSHLYQVKAVGEEMGIGFLGLGFQPKWALSDIPIMPKGRYEIMRNYMPKVGTLGLDMMFRTCTVQVNLDFSSEQDMIRKFRAGLALQPIATAIFANSPFKEGKPNGFLSLRSHIWTDTDNNRAGMLPFVFDDSFGFEQYVDYALEVPMYFVYRNKKYIDCTGMSFRDFMQGKLPQAPGELPTLTDWENHLTTIFPEVRLKRYLEMRGADGGPWRRLCALPAFWVGLLYDEESLQSILDMTFDWTKEEREMLRRKVPSTGLKTPFRDGYVRDLAEEVLKLAKNGLERRGYKEVGFLREVDEVVRTGVTPAERLLSPYETKWQRNVDHVFEHLLY

SEQ ID NO: 11 - DNA, 1503 bp, Zea mays
atggccgtggcgtcgcggctcgcggtcacgcgtgtgtcgccggcggacggcgcgcgccccgcggcggcggcggggaggaggagtgggctcgcggtggttcggctcccgccgaccgacagcagggggagaaggaggaggcgctgcggggccgtcgcggccagccccccgacggaggaggtcgtgcagatgacggagccgctcaccaaggaggacctcgtcgcctacctcgtctccgggtgcaagcccaaggagaactggagaattggcacggagcatgaaaagtttggttttgaagtcgacacattacgccctataaaatatgatcagattcgtgacatactgaacgggctcgctgagagatttgattgggagaagataatggaaggaaacattgttatcggcctcaagcagggaaagcaaagcatctcactagaacctggaggccaatttgaacttagtggcgctcctctcgaaacgttacatcaaacttgtgctgaggtctactcacatctatatcaggtcaaagcagtcggagaagaaatgggaataggatttcttgggcttggctttcagccaaaatgggcactgagtgacataccaataatgccaaagggaagatacgaaataatgaggaattacatgcctaaagttggtactcttggccttgatatgatgttccggacatgtactgtgcaggttaatcttgacttcagttcagaacaggatatgataaggaaatttcgcgctggcctcgctttgcagcctattgcaactgcaatatttgccaattctcccttcaaagaaggaaaaccaaatggatttctcagcctaaggagccatatctggacagataccgataacaaccgtgcagggatgctcccttttgtctttgacaactcatttgggtttgagcaatatgtggattatgcattagatgtccccatgtattttgtgtaccgaaataataagtatattgactgcaccggaatgtcatttcgggattttatgcaaggaaagctccgacaagctcctggggagttgcctactcttaatgattgggagaaccatctaacaacaatttttcctgaggttaggttaaagagataccttgagatgagaggtgctgatggtggcccatggaggagattgtgtgcgctgcctgcattttgggttgggctgctgtacgatgaggaatcattacaaagcattttagacatgacttttgactggacacaggaggaaagagagatgctaagacataaggtaccgttgactggtctgaagacaccatttcgcgatggatatgttagagatttagccgaggaagttctaaaactggccaagaatggattggaaagaagaggatacaaggaggtcggtttccttagagaggttgacgaagtggtgaggacaggagtgacacctgccgagagacttctgcatctgtacgagacgaagtggcaacgcaacgtagaccatgttttcgagcacttgctatactga

SEQ ID NO: 12 - protein, 500 aa, Zea mays
MAVASRLAVTRVSPADGARPAAAAGRRSGLAVVRLPPTDSRGRRRRRCGAVAASPPTEEVVQMTEPLTKEDLVAYLVSGCKPKENWRIGTEHEKFGFEVDTLRPIKYDQIRDILNGLAERFDWEKIMEGNIVIGLKQGKQSISLEPGGQFELSGAPLETLHQTCAEVYSHLYQVKAVGEEMGIGFLGLGFQPKWALSDIPIMPKGRYEIMRNYMPKVGTLGLDMMFRTCTVQVNLDFSSEQDMIRKFRAGLALQPIATAIFANSPFKEGKPNGFLSLRSHIWTDTDNNRAGMLPFVFDNSFGFEQYVDYALDVPMYFVYRNNKYIDCTGMSFRDFMQGKLRQAPGELPTLNDWENHLTTIFPEVRLKRYLEMRGADGGPWRRLCALPAFWVGLLYDEESLQSILDMTFDWTQEEREMLRHKVPLTGLKTPFRDGYVRDLAEEVLKLAKNGLERRGYKEVGFLREVDEVVRTGVTPAERLLHLYETKWQRNVDHVFEHLLY

SEQ ID NO: 13 - DNA, 1350 bp, Zea mays
atggccagccccccgacggaggaggtcgtgcagatgacggagccgctcaccaaggaggacctcgtcgcctacctcgtctccgggtgcaagcccaaggagaactggagaattggcacggagcatgaaaagtttggttttgaagtcgacacattacgccctataaaatatgatcagattcgtgacatactgaacgggctcgctgagagatttgattgggagaagataatggaaggaaacattgttatcggcctcaagcagggaaagcaaagcatctcactagaacctggaggccaatttgaacttagtggcgctcctctcgaaacgttacatcaaacttgtgctgaggtctactcacatctatatcaggtcaaagcagtcggagaagaaatgggaataggatttcttgggcttggctttcagccaaaatgggcactgagtgacataccaataatgccaaagggaagatacgaaataatgaggaattacatgcctaaagttggtactcttggccttgatatgatgttccggacatgtactgtgcaggttaatcttgacttcagttcagaacaggatatgataaggaaatttcgcgctggcctcgctttgcagcctattgcaactgcaatatttgccaattctcccttcaaagaaggaaaaccaaatggatttctcagcctaaggagccatatctggacagataccgataacaaccgtgcagggatgctcccttttgtctttgacaactcatttgggtttgagcaatatgtggattatgcattagatgtccccatgtattttgtgtaccgaaataataagtatattgactgcaccggaatgtcatttcgggattttatgcaaggaaagctccgacaagctcctggggagttgcctactcttaatgattgggagaaccatctaacaacaatttttcctgaggttaggttaaagagataccttgagatgagaggtgctgatggtggcccatggaggagattgtgtgcgctgcctgcattttgggttgggctgctgtacgatgaggaatcattacaaagcattttagacatgacttttgactggacacaggaggaaagagagatgctaagacataaggtaccgttgactggtctgaagacaccatttcgcgatggatatgttagagatttagccgaggaagttctaaaactggccaagaatggattggaaagaagaggatacaaggaggtcggtttccttagagaggttgacgaagtggtgaggacaggagtgacacctgccgagagacttctgcatctgtacgagacgaagtggcaacgcaacgtagaccatgttttcgagcacttgctatactga

SEQ ID NO: 14 - protein, 449 aa, Zea mays
MASPPTEEVVQMTEPLTKEDLVAYLVSGCKPKENWRIGTEHEKFGFEVDTLRPIKYDQIRDILNGLAERFDWEKIMEGNIVIGLKQGKQSISLEPGGQFELSGAPLETLHQTCAEVYSHLYQVKAVGEEMGIGFLGLGFQPKWALSDIPIMPKGRYEIMRNYMPKVGTLGLDMMFRTCTVQVNLDFSSEQDMIRKFRAGLALQPIATAIFANSPFKEGKPNGFLSLRSHIWTDTDNNRAGMLPFVFDNSFGFEQYVDYALDVPMYFVYRNNKYIDCTGMSFRDFMQGKLRQAPGELPTLNDWENHLTTIFPEVRLKRYLEMRGADGGPWRRLCALPAFWVGLLYDEESLQSILDMTFDWTQEEREMLRHKVPLTGLKTPFRDGYVRDLAEEVLKLAKNGLERRGYKEVGFLREVDEVVRTGVTPAERLLHLYETKWQRNVDHVFEHLLY

SEQ ID NO: 15 - DNA, 1848 bp, Helianthus annuus
gccttccgccaccggataaaaagaaatggtattaatgtctcagacgagtccatcacatggcattcgtactgagattttacagtctaaatctggatatacttcactttttagtggggcaaacaacacaaatgcatttagacacaggacctcaaccgttgcgtttccacggaattcctcaaaatcttcccaaaatatgcatgtagatgccattggtgagaaagtcaaaaggggcaataaagtaattgttgctgcaagcccccccacagaggacgcggttgttgctacagaaccacttacaaaagaagatcttgtgggataccttgcttctggctgcaagcctaaggaaaactggagaataggaactgaacatgaaaaattcggttttgatcttaaaacattgcgtcctatgacgtatgaacaaattgctcatctgctaaatgctatttccgagagatttggttgggacaaagtcatggaaggcgacaatataattggacttcaacagggaaaacaaagtatatctctggaacctggtggtcgtggtcagtttgagctgagtggtgcgcctcttgaaactctccatcaaacttgtgcagaagttaattcacacctttaccaggttaaagctgttgctgaagagatgggaatcgggtttattggaattggttttcaacctaaatgggaaaggaaagatataccagtaatgcccaagggaagatacgagattatgcggaattacatgcctaaagttggttctcttggacttgacatgatgttcaggacatgtactgttcaggttaacttggacttctcttctgaagctgacatgataagaaaattccgtgctggtcttgctttacaacctatcgctacagcactgtttgctaattcgccatttacagaaggaaagccgaatggttatctcagcatgaggagccaaatatggacagacaccgataataatcgttctggaatgcttccttttgtctttgatgattcctttggatttgagcaatatgttgaatatgctctcgatgtccctatgtattttgtttatcggaagaaaaagtatatcgactgtgcgggattgtccttcagggacttcctcgccggaaaactcccttcgattcccggagaatatccaactctcaatgattgggagaatcacctcacaacaatatttccggaggtgagacttaaaaggtacttggaaacgaggggtgctgatggagggccatggaggaggttatgtgcattgcctgctttttgggtgggcatattgtatgatgatatttctctgcaaaatgttttggacatgacagccgattggactcaaggcgaaagacagatgttgagaaataaggtgcctgtaactggtctgaaaaccccattccgtgatggattgctgaaacatgttgctgaagaagttttgcagttagcaaaggatggcctggagagaagaggatataaagaaacagggttcttaaatgaagtagcagaggtggtcagaacaggtttaacaccagcagagaagcttctggaactgtatcatggaaaatggggacaaaatgttgaccctgtatttgaggaattactctattaagatattcatgttgttgtccatatttatgtaatgaataaggtgtgtgctgcgtgcatgaagtgatcatggacttagtggccggtgtgatcagtaatgcaacaagacgcatttagtgagtgatactaccattcgaaacttctgaattgtaggcttctttgttcacctcagatttacataaaataagttttgtatttgtatttctttcttttaagacaccattctactggtctattatcaagcttaat

SEQ ID NO: 16 - protein, 525 aa, Helianthus annuus
MVLMSQTSPSHGIRTEILQSKSGYTSLFSGANNTNAFRHRTSTVAFPRNSSKSSQNMHVDAIGEKVKRGNKVIVAASPPTEDAVVATEPLTKEDLVGYLASGCKPKENWRIGTEHEKFGFDLKTLRPMTYEQIAHLLNAISERFGWDKVMEGDNIIGLQQGKQSISLEPGGRGQFELSGAPLETLHQTCAEVNSHLYQVKAVAEEMGIGFIGIGFQPKWERKDIPVMPKGRYEIMRNYMPKVGSLGLDMMFRTCTVQVNLDFSSEADMIRKFRAGLALQPIATALFANSPFTEGKPNGYLSMRSQIWTDTDNNRSGMLPFVFDDSFGFEQYVEYALDVPMYFVYRKKKYIDCAGLSFRDFLAGKLPSIPGEYPTLNDWENHLTTIFPEVRLKRYLETRGADGGPWRRLCALPAFWVGILYDDISLQNVLDMTADWTQGERQMLRNKVPVTGLKTPFRDGLLKHVAEEVLQLAKDGLERRGYKETGFLNEVAEVVRTGLTPAEKLLELYHGKWGQNVDPVFEELLY

SEQ ID NO: 17 - DNA, 1356 bp, Helianthus annuus
atggcaagcccccccacagaggacgcggttgttgctacagaaccacttacaaaagaagatcttgtgggataccttgcttctggctgcaagcctaaggaaaactggagaataggaactgaacatgaaaaattcggttttgatcttaaaacattgcgtcctatgacgtatgaacaaattgctcatctgctaaatgctatttccgagagatttggttgggacaaagtcatggaaggcgacaatataattggacttcaacagggaaaacaaagtatatctctggaacctggtggtcgtggtcagtttgagctgagtggtgcgcctcttgaaactctccatcaaacttgtgcagaagttaattcacacctttaccaggttaaagctgttgctgaagagatgggaatcgggtttattggaattggttttcaacctaaatgggaaaggaaagatataccagtaatgcccaagggaagatacgagattatgcggaattacatgcctaaagttggttctcttggacttgacatgatgttcaggacatgtactgttcaggttaacttggacttctcttctgaagctgacatgataagaaaattccgtgctggtcttgctttacaacctatcgctacagcactgtttgctaattcgccatttacagaaggaaagccgaatggttatctcagcatgaggagccaaatatggacagacaccgataataatcgttctggaatgcttccttttgtctttgatgattcctttggatttgagcaatatgttgaatatgctctcgatgtccctatgtattttgtttatcggaagaaaaagtatatcgactgtgcgggattgtccttcagggacttcctcgccggaaaactcccttcgattcccggagaatatccaactctcaatgattgggagaatcacctcacaacaatatttccggaggtgagacttaaaaggtacttggaaacgaggggtgctgatggagggccatggaggaggttatgtgcattgcctgctttttgggtgggcatattgtatgatgatatttctctgcaaaatgttttggacatgacagccgattggactcaaggcgaaagacagatgttgagaaataaggtgcctgtaactggtctgaaaaccccattccgtgatggattgctgaaacatgttgctgaagaagttttgcagttagcaaaggatggcctggagagaagaggatataaagaaacagggttcttaaatgaagtagcagaggtggtcagaacaggtttaacaccagcagagaagcttctggaactgtatcatggaaaatggggacaaaatgttgaccctgtatttgaggaattactctattaa

SEQ ID NO: 18 - protein, 451 aa, Helianthus annuus
MASPPTEDAVVATEPLTKEDLVGYLASGCKPKENWRIGTEHEKFGFDLKTLRPMTYEQIAHLLNAISERFGWDKVMEGDNIIGLQQGKQSISLEPGGRGQFELSGAPLETLHQTCAEVNSHLYQVKAVAEEMGIGFIGIGFQPKWERKDIPVMPKGRYEIMRNYMPKVGSLGLDMMFRTCTVQVNLDFSSEADMIRKFRAGLALQPIATALFANSPFTEGKPNGYLSMRSQIWTDTDNNRSGMLPFVFDDSFGFEQYVEYALDVPMYFVYRKKKYIDCAGLSFRDFLAGKLPSIPGEYPTLNDWENHLTTIFPEVRLKRYLETRGADGGPWRRLCALPAFWVGILYDDISLQNVLDMTADWTQGERQMLRNKVPVTGLKTPFRDGLLKHVAEEVLQLAKDGLERRGYKETGFLNEVAEVVRTGLTPAEKLLELYHGKWGQNVDPVFEELLY

SEQ ID NO: 19 - DNA, 1864 bp, Arabidopsis thaliana
ctcaatctccgtcaagcttgacgaatttcaggagctatatataccatggcgctcttgtctcaagcaggaggatcatacactgttgttccttctggagtttgttcaaagactggaactaaagctgttgtttctggtggcgtgaggaatttggatgttttgaggatgaaagaagcttttggtagctccaactctaggagtctatctaccaaatcaatgcttctccattctgttaagaggagtaagagagggcatcaattgattgttgcggcaagtcctccaacggaagaggctgtagttgcaactgagccgttgacgagagaggatctcattgcctatcttgcctctggatgcaaaacaaaggacaaatatagaataggtacagaacatgagaaatttggttttgaggtcaatactttgcgccctatgaagtatgatcaaatagccgagcttcttaatggtatcgctgaaagatttgaatgggaaaaagtaatggaaggtgacaagatcattggtctgaagcagggaaagcaaagcatttcacttgaacctgggggtcagttcgagcttagtggtgcacctcttgagactttgcatcaaacttgtgctgaagtcaattcacatctttatcaggtaaaagcagttgctgaggaaatgggaattggtttcttaggaatcggcttccagcccaaatggcgtcgggaggatatacccatcatgccaaaggggagatacgacattatgagaaactacatgccgaaagttggtacccttggtcttgatatgatgctccgaacgtgtactgttcaggttaatctggattttagctcagaagctgatatgatcaggaagtttcgtgctggtcttgctttacaacctatagcaacggctctatttgcgaattccccttttacagaaggaaagccaaacggatttctcagcatgagaagccacatatggacagacactgacaaggaccgcacaggaatgctaccatttgttttcgatgactcttttgggtttgagcagtatgttgactacgcactcgatgtccctatgtactttgcctacagaaagaacaaatacatcgactgtactggaatgacatttcggcaattcttggctggaaaacttccctgtctccctggtgaactgccttcatataatgattgggaaaaccatctgacaacaatattcccagaggttcggttgaagagatacttggagatgagaggtgctgatggaggtccctggaggaggctgtgtgccctgccagctttctgggtgggtttattatatgatgatgatagtctccaagctatcctggatctgacagctgactggactccagcagagagagagatgctaaggaacaaagtcccagttactggcttaaagactccttttagggatggtttgttaaagcatgtcgctgaagatgtcctgaaactcgcaaaggatggtttagagcgcagaggctacaaggaagccggtttcttgaacgcagtcgatgaagtggtcagaacaggagttacgcctgcggagaagctcttggagatgtacaatggagaatggggacaaagcgtagatcccgtgttcgaagagctgctgtactaagagaatgggacgtgaacaaaaggtgtctataaacctctgggtgtgagtttatgctatctgaagaactcgagtctcaggaataaggatttttttttttggttgtaatcggattttaaaaactgattttgttttagaaattcgaagcattgaaaatcagaagaaaaattgtatgtactaaacgatttcggtgtgggaaatcgtttgggagggtgtgtttggatctttgaataaattacccatttttcttgtc

SEQ ID NO: 20 - protein, 522 aa, Arabidopsis thaliana
MALLSQAGGSYTVVPSGVCSKTGTKAVVSGGVRNLDVLRMKEAFGSSNSRSLSTKSMLLHSVKRSKRGHQLIVAASPPTEEAVVATEPLTREDLIAYLASGCKTKDKYRIGTEHEKFGFEVNTLRPMKYDQIAELLNGIAERFEWEKVMEGDKIIGLKQGKQSISLEPGGQFELSGAPLETLHQTCAEVNSHLYQVKAVAEEMGIGFLGIGFQPKWRREDIPIMPKGRYDIMRNYMPKVGTLGLDMMLRTCTVQVNLDFSSEADMIRKFRAGLALQPIATALFANSPFTEGKPNGFLSMRSHIWTDTDKDRTGMLPFVFDDSFGFEQYVDYALDVPMYFAYRKNKYIDCTGMTFRQFLAGKLPCLPGELPSYNDWENHLTTIFPEVRLKRYLEMRGADGGPWRRLCALPAFWVGLLYDDDSLQAILDLTADWTPAEREMLRNKVPVTGLKTPFRDGLLKHVAEDVLKLAKDGLERRGYKEAGFLNAVDEVVRTGVTPAEKLLEMYNGEWGQSVDPVFEELLY

SEQ ID NO: 21 - DNA, 1347 bp, Arabidopsis thaliana
atggcaagtcctccaacggaagaggctgtagttgcaactgagccgttgacgagagaggatctcattgcctatcttgcctctggatgcaaaacaaaggacaaatatagaataggtacagaacatgagaaatttggttttgaggtcaatactttgcgccctatgaagtatgatcaaatagccgagcttcttaatggtatcgctgaaagatttgaatgggaaaaagtaatggaaggtgacaagatcattggtctgaagcagggaaagcaaagcatttcacttgaacctgggggtcagttcgagcttagtggtgcacctcttgagactttgcatcaaacttgtgctgaagtcaattcacatctttatcaggtaaaagcagttgctgaggaaatgggaattggtttcttaggaattggcttccagcccaaatggcgtcgggaggatatacccatcatgccaaaggggagatacgacattatgagaaactacatgccgaaagttggtacccttggtcttgatatgatgctccgaacgtgtactgttcaggttaatctggattttagctcagaagctgatatgatcaggaagtttcgtgctggtcttgctttacaacctatagcaacggctctatttgcgaattccccttttacagaaggaaagccaaacggatttctcagcatgagaagccacatatggacagacactgacaaggaccgcacaggaatgctaccatttgttttcgatgactcttttgggtttgagcagtatgttgactacgcactcgatgtccctatgtactttgcctacagaaagaacaaatacatcgactgtactggaatgacatttcggcaattcttggctggaaaacttccctgtctccctggtgaactgccttcatataatgattgggaaaaccatctgacaacaatattcccagaggttcggttgaagagatacttggagatgagaggtgctgatggaggtccctggaggaggctgtgtgccctgccagctttctgggtgggtttattatatgatgatgatagtctccaagctatcctggatctgacagctgactggactccagcagagagagagatgctaaggaacaaagtcccagttactggcttaaagactccttttagggatggtttgttaaagcatgtcgctgaagatgtcctgaaactcgcaaaggatggtttagagcgcagaggctacaaggaagccggtttcttgaacgcagtcgatgaagtggtcagaacaggagttacgcctgcggagaagctcttggagatgtacaatggagaatggggacaaagcgtagatcccgtgttcgaagagctgctgtac

SEQ ID NO: 22 - protein, 449 aa, Arabidopsis thaliana
MASPPTEEAVVATEPLTREDLIAYLASGCKTKDKYRIGTEHEKFGFEVNTLRPMKYDQIAELLNGIAERFEWEKVMEGDKIIGLKQGKQSISLEPGGQFELSGAPLETLHQTCAEVNSHLYQVKAVAEEMGIGFLGIGFQPKWRREDIPIMPKGRYDIMRNYMPKVGTLGLDMMLRTCTVQVNLDFSSEADMIRKFRAGLALQPIATALFANSPFTEGKPNGFLSMRSHIWTDTDKDRTGMLPFVFDDSFGFEQYVDYALDVPMYFAYRKNKYIDCTGMTFRQFLAGKLPCLPGELPSYNDWENHLTTIFPEVRLKRYLEMRGADGGPWRRLCALPAFWVGLLYDDDSLQAILDLTADWTPAEREMLRNKVPVTGLKTPFRDGLLKHVAEDVLKLAKDGLERRGYKEAGFLNAVDEVVRTGVTPAEKLLEMYNGEWGQSVDPVFEELLY

SEQ ID NO: 23 - protein, 508 aa, Phaseolus vulgaris
MAVLGRTTAAYTHRHLPRRHFDGQTKASAPNTFSCSNWDSAKKLSPTQRIVTRGGRVIVAASPPTEDAVVATDPLTKQDLVDYLASGCKPREKWRIGTEHEKFGFEFGSLRPMKYEQIAELLNGIAERFDWDKIMEGDKIIGLKQGKQSISLEPGGQFELSGAPLETLHQTCAEVNSHLYQVKAVAEEMEIGFLGIGFQPKWGIEDIPVMPKGRYDIMRNYMPKVGSLGLDIMFRTCTVQVNLDFSSEADMIRKFRAGLALQPIATALFANSPFKEGKPNGFVSMRSHIWTDTDKDRTGMLPFVFDDSFGFEQYVDYALDVPMYFVYRKHRYIDCTGKTFRDFLAGRLPCIPGELPTLNDWENHLTTIFPEVRLKRYLEMRGADGGPWRRLCALPALWVGLLYDEASLQSLLDLTADWTPEERQMLRNKVPVTGLKTPFRDGLLKHVAEDVLQLAKDGLERRGFKESGFLNEVAEVVRTGVTPAERLLELYHGKWEQSVDHVFEELLY

SEQ ID NO: 24 - protein, 438 aa, Zea mays
MTEPLTKEDLVAYLVSGCKPKENWRIGTEHEKFGFEVDTLRPLKYDQIRDILNGLAERFDWDKIMEKNNVIGLKQGKQSISLEPGGQFELSGAPLETLHQTCAEVNSHLYQVKAVGEEMGIGFLGLGFQPKWALSDIPIMPKGRYEIMRNYMPKVGTLGLDMMFRTCTVQVNLDFSSEQDMIRKFRAGLALQPIATAIFANSPFKEGKPNGFLSLRSHIWTDTDNNRAGMLPFVFDDSFGFEQYVDYALEVPMYFVYRNKKYIDCTGMSFRDFMQGKLPQAPGELPTLTDWENHLTTIFPEVRLKRYLEMRGADGGPWRRLCALPAFWVGLLYDEESLQSILDMTFDWTKEEREMLRRKVPSTGLKTPFRDGYVRDLAEEVLKLAKNGLERRGYKEVGFLREVDEVVRTGVTPAERLLSPYETKWQRNVDHVFEHLLY

SEQ ID NO: 25 - protein, 523 aa, Zinnia violacea
MVLMSQASPSHGIHAEILQSKSGYSSLLNGASNTNAFRHQTSKVAFSRNYLKYTQAMHVDAVGGNFKRGNKVIVAASPPTEDAVVATEPLTKEDLVGYLASGCKPKENWRIGTEHEKFGFDLKTLRPMTYEQIAHLLNAISERFDWEKVMEGDNIIGLKQGKQSISLEPGGQFELSGAPLETLHQTCAEVNSHLYQVKAVAEEMGIGFIGIGFQPKWERKDIPIMPKGRYEIMRNYMPKVGSLGLDMMFRTCTVQVNLDFSSEADMIRKFRAGLALQPIATALFANSPFTEGKPNGYLSMRSQIWTDTDNDRSGMLPFVFDDSFGFEQYVEYALDVPMYFVYRKKKYIDCAGLSFRDFLAGKLPPIPGEYPTLNDWENHLTTIFPEVRLKRYLEMRGADGGPWRRLCALPAFWVGVLYDDISLQNVLDMTADWTQEERQMLRNKVPVAGLKTPFRDGLLKHVAEEVLKFAKDGLERRGYKETGFLNEVAEVVRTGLTPAEKLLELYHGKWGQSVDPVFEELLY

SEQ ID NO: 26 - protein, 505 aa, Glycine max
  misc_feature (99)..(99): Xaa can be any naturally occurring amino acid
MAVVSRSATTYTRHYLIRHEFDRKTKTCVANNSLCYSAKKAPPPQRIVGGRRVIVAASPPTEDAVVATDPLTKQDLVDYLASGCKPKDKWRIGTEHEKXGFEIGSLRPMKYDQIAELLNGIAERFDWDKVMEGDKIIGLKQGKQSISLEPGGQFELSGAPLETLHQTCAEVNSHLYQVKAVAEEMGIGFLGIGFQPKWGIKDIPIMPKGRYDIMRNYMPKVGSLGLDMMFRTCTVQVNLDFSSEADMIKKFRAGLALQPIATALFANSPFKEGKPNGFVSMRSHIWTDTDKDRTGMLPFVFDDSFGFEQYVDYAXLDVPMYYVFRKHRYIDCTGKTFRDFLAGRLPCIPGELPTLNDWENHLTTIFALPAFRVELLNDEADGGPWRRLCALPAFWVGLLYDELSLKSVLDMTADWTPEERQMLRNKVPVTGLKTPFRDGLLKHVAEDVLKLAKDGLERRGFKESGFLNEVAEVVRTGVTPAERLLELYHGKWEQSVDHVFEELLY

SEQ ID NO: 27 - protein, 492 aa, Oryza sativa
MAVASRLAVARVAPDGGAAGRRRRRGRPVVAVPTAGRGRGGAVAASPPTEEAVQMTEPLTKEDLVAYLVSGCKPKENWRIGTEHEKFGFEVDTLRPIKYDQIRDILNGLAERFDWDKIVEENNVIGLKQGKQSISLEPGGQFELSGAPLETLHQTCAEVNSHLYQVKAVGEEMGIGFLGIGFQPKWALSDIPIMPKGRYEIMRNYMPKVGSLGLDMMFRTCTVQVNLDFSSEQDMIRKFRTGLALQPIATAIFANSPFKEGKPNGYLSLRSHIWTDTDNNRSGMLPFVFDDSFGFERYVDYALDIPMYFVYRNKKYIDCTGMSFRDFMVGKLPQAPGELPTLNDWENHLTTIFPEVRLKRYLEMRGADGGPWRRLCALPVFWVGLLYDEESLQSISDMTSDWTNEEREMLRRKVPVTGLKTPFRDGYVRDLAEEILQLSKNGLERRGYKEVGFLREVDAVISSGVTPAERLLNLYETKWQRSVDPVFQELLY

SEQ ID NO: 28 - protein, 522 aa, Arabidopsis thaliana
MALLSQAGGSYTVVPSGVCSKAGTKAVVSGGVRNLDVLRMKEAFGSSYSRSLSTKSMLLHSVKRSKRGHQLIVAASPPTEEAVVATEPLTREDLIAYLASGCKTKDKYRIGTEHEKFGFEVNTLRPMKYDQIAELLNGIAERFEWEKVMEGDKIIGLKQGKQSISLEPGGQFELSGAPLETLHQTCAEVNSHLYQVKAVAEEMGIGFLGIGFQPKWRREDIPIMPKGRYDIMRNYMPKVGTLGLDMMLRTCTVQVNLDFSSEADMIRKFRAGLALQPIATALFANSPFTEGKPNGFLSMRSHIWTDTDKDRTGMLPFVFDDSFGFEQYVDYALDVPMYFAYRKNKYIDCTGMTFRQFLAGKLPCLPGELPSYNDWENHLTTIFPEVRLKRYLEMRGADGGPWRRLCALPAFWVGLLYDDDSLQAILDLTADWTPAEREMLRNKVPVTGLKTPFRDGLLKHVAEDVLKLAKDGLERRGYKEAGFLNAVDEVVRTGVTPAEKLLEMYNGEWGQSVDPVFEELLY

SEQ ID NO: 29 - DNA, 1512 bp, Zea mays
atggcggtggcgtcgcggctggcggtcgcgcgggtgtcgccggacggcgcgcgccccgcggcggcggcggcggcaggggggagggggaggagcgggctcgcggcggttcggctcccgtcgaccgccggttgggtgaggaggagggggcgcggcggggccgtcgcggccagccctcccacggaggaggccgtgcagatgacggagccgctcaccaaggaggacctcgtcgcctacctcgtctccgggtgcaagcccaaggagaattggagaattgggacggagcacgaaaagttcggtttcgaagtcgacactttacgccctataaaatatgatcagattcgtgacatactgaacggtcttgctgagagatttgattgggacaagataatggaagaaaacaatgttatcggtctcaagcagggaaagcaaagcatctcactagaacctggaggccaatttgaacttagtggcgctcctctcgaaacattacatcaaacttgtgctgaggtcaattcgcatctttatcaggttaaagcagttggagaggaaatgggaataggatttcttgggcttggctttcagccaaaatgggcactgagtgacataccaataatgccaaagggaagatacgaaataatgaggaattacatgcctaaagttggtactcttggccttgatatgatgttccggacatgtactgtgcaggttaatcttgacttcagttcagaacaggatatgataaggaaatttcgcgctggcctcgctttgcagcctattgcaactgcaatatttgccaattctccgttcaaagaaggaaaaccaaatggatttctcagcttaaggagccatatctggacagatactgataataatcgtgcagggatgctcccttttgtctttgacgactcatttgggtttgagcaatatgtggactatgcattagaagtccccatgtattttgtgtaccgaaataaaaagtatattgactgcaccggaatgtcgtttcgggattttatgcaaggaaagcttccacaggctcctggggagttgcccactcttaacgattgggagaaccatctaacaacaatttttcctgaggttaggctaaagaggtaccttgagatgagaggtgctgatggtggcccatggaggagattgtgtgcgttgcctgcattttgggttgggctgctgtacgacgaggaatcgttacaaagcattttagacatgacttttgattggacaaaggaggaaagagagatgctaagacggaaggtaccatcgactggtttgaagacgccgtttcgtgatggatatgtaagagatttagctgaggaagttctaaaactggccaaggttggactggaaagaagagggtacaaggaggttggtttccttagagaggtcgacgaagtagtgagaacaggagtgacgcctgcggagaggctgctgaacctgtacgagaccaagtggcaacgcaacgtcgaccatgttttcgagcatttgttatactga

SEQ ID NO: 30 - protein, 503 aa, Zea mays
MAVASRLAVARVSPDGARPAAAAAAGGRGRSGLAAVRLPSTAGWVRRRGRGGAVAASPPTEEAVQMTEPLTKEDLVAYLVSGCKPKENWRIGTEHEKFGFEVDTLRPIKYDQIRDILNGLAERFDWDKIMEENNVIGLKQGKQSISLEPGGQFELSGAPLETLHQTCAEVNSHLYQVKAVGEEMGIGFLGLGFQPKWALSDIPIMPKGRYEIMRNYMPKVGTLGLDMMFRTCTVQVNLDFSSEQDMIRKFRAGLALQPIATAIFANSPFKEGKPNGFLSLRSHIWTDTDNNRAGMLPFVFDDSFGFEQYVDYALEVPMYFVYRNKKYIDCTGMSFRDFMQGKLPQAPGELPTLNDWENHLTTIFPEVRLKRYLEMRGADGGPWRRLCALPAFWVGLLYDEESLQSILDMTFDWTKEEREMLRRKVPSTGLKTPFRDGYVRDLAEEVLKLAKVGLERRGYKEVGFLREVDEVVRTGVTPAERLLNLYETKWQRNVDHVFEHLLY

SEQ ID NO: 31 - DNA, 1350 bp, Zea mays
atggccagccctcccacggaggaggccgtgcagatgacggagccgctcaccaaggaggacctcgtcgcctacctcgtctccgggtgcaagcccaaggagaattggagaattgggacggagcacgaaaagttcggtttcgaagtcgacactttacgccctataaaatatgatcagattcgtgacatactgaacggtcttgctgagagatttgattgggacaagataatggaagaaaacaatgttatcggtctcaagcagggaaagcaaagcatctcactagaacctggaggccaatttgaacttagtggcgctcctctcgaaacattacatcaaacttgtgctgaggtcaattcgcatctttatcaggttaaagcagttggagaggaaatgggaataggatttcttgggcttggctttcagccaaaatgggcactgagtgacataccaataatgccaaagggaagatacgaaataatgaggaattacatgcctaaagttggtactcttggccttgatatgatgttccggacatgtactgtgcaggttaatcttgacttcagttcagaacaggatatgataaggaaatttcgcgctggcctcgctttgcagcctattgcaactgcaatatttgccaattctccgttcaaagaaggaaaaccaaatggatttctcagcttaaggagccatatctggacagatactgataataatcgtgcagggatgctcccttttgtctttgacgactcatttgggtttgagcaatatgtggactatgcattagaagtccccatgtattttgtgtaccgaaataaaaagtatattgactgcaccggaatgtcgtttcgggattttatgcaaggaaagcttccacaggctcctggggagttgcccactcttaacgattgggagaaccatctaacaacaatttttcctgaggttaggctaaagaggtaccttgagatgagaggtgctgatggtggcccatggaggagattgtgtgcgttgcctgcattttgggttgggctgctgtacgacgaggaatcgttacaaagcattttagacatgacttttgattggacaaaggaggaaagagagatgctaagacggaaggtaccatcgactggtttgaagacgccgtttcgtgatggatatgtaagagatttagctgaggaagttctaaaactggccaaggttggactggaaagaagagggtacaaggaggttggtttccttagagaggtcgacgaagtagtgagaacaggagtgacgcctgcggagaggctgctgaacctgtacgagaccaagtggcaacgcaacgtcgaccatgttttcgagcatttgttatactga

SEQ ID NO: 32 - protein, 449 aa, Zea mays
MASPPTEEAVQMTEPLTKEDLVAYLVSGCKPKENWRIGTEHEKFGFEVDTLRPIKYDQIRDILNGLAERFDWDKIMEENNVIGLKQGKQSISLEPGGQFELSGAPLETLHQTCAEVNSHLYQVKAVGEEMGIGFLGLGFQPKWALSDIPIMPKGRYEIMRNYMPKVGTLGLDMMFRTCTVQVNLDFSSEQDMIRKFRAGLALQPIATAIFANSPFKEGKPNGFLSLRSHIWTDTDNNRAGMLPFVFDDSFGFEQYVDYALEVPMYFVYRNKKYIDCTGMSFRDFMQGKLPQAPGELPTLNDWENHLTTIFPEVRLKRYLEMRGADGGPWRRLCALPAFWVGLLYDEESLQSILDMTFDWTKEEREMLRRKVPSTGLKTPFRDGYVRDLAEEVLKLAKVGLERRGYKEVGFLREVDEVVRTGVTPAERLLNLYETKWQRNVDHVFEHLLY

SEQ ID NO: 33 - DNA, 25 bp, Artificial Sequence
  sequence of attB1 site
acaagtttgtacaaaaaagcaggct

SEQ ID NO: 34 - DNA, 25 bp, Artificial Sequence
  sequence of attB2 site
accactttgtacaagaaagctgggt

SEQ ID NO: 35 - DNA, 54 bp, Artificial Sequence
  sequence of the VC062 primer
ttaaacaagtttgtacaaaaaagcaggctgcaattaaccctcactaaagggaac

SEQ ID NO: 36 - DNA, 53 bp, Artificial Sequence
  sequence of the VC063 primer
ttaaaccactttgtacaagaaagctgggtgcgtaatacgactcactatagggc

SEQ ID NO: 37 - DNA, 24 bp, Artificial Sequence
  forward primer GM-GSH-F3
ccatgggaattggatttttgggga

SEQ ID NO: 38 - DNA, 28 bp, Artificial Sequence
  reverse primer GM-GSH-R1
ttcgaagtatatgagaagcctcaaggca

SEQ ID NO: 39 - DNA, 18 bp, Artificial Sequence
  primer PHN_131845
gccatggctgtcgtttcg

SEQ ID NO: 40 - DNA, 28 bp, Artificial Sequence
  primer PHN_131846
ttcgaagtatatgagaagcctcaaggca

SEQ ID NO: 41 - DNA, 1660 bp, Glycine max
gccatggctgtcgtttcgcgaagtgcgacgacctatacgcgccactacttaatacgacacgagtttgataggaaaacgaaaacctgcgttgccaataatagtttgtgttactctgctaagaaggctcctccaccgcagaggattgttggtggccgtagagtgattgttgctgcgagccctcccaccgaagacgctgtagttgccactgaccctctcacgaagcaggatctcgtcgattatcttgcctccggttgcaagcccaaggataaatggagaataggtactgaacatgagaagtttggttttgagattggaagcttgcgtcctatgaagtatgaccaaatagcagaattgctgaatggcattgctgagaggtttgactgggataaagtaatggaaggtgataaaattattggactcaaacaggggaagcagagcatatcattggagcctggtggtcagtttgaacttagtggagctcctcttgaaaccttgcatcagacttgtgctgaagttaattcccacctttatcaggttaaagctgttgctgaggaaatgggaattggatttttggggattggtttccagccaaagtggggaatcaaagacatacctataatgccaaagggaagatacgacatcatgaggaactacatgcctaaagttggctctcttgggcttgacatgatgttcaggacatgcactgtgcaggtcaatctggactttagttctgaagctgacatgatcaagaaatttcgtgcaggccttgctttgcagccgatagcaacggctctttttgcaaattcaccctttaaagagggaaagccaaatggttttgtcagtatgagaagccatatttggactgatactgataaggaccgcacaggcatgctgccttttgtttttgatgactcttttgggtttgagcaatatgttgattatgctcttgatgttcctatgtattttgtctatcggaaaaacagatatatcgactgcactggaaagaccttcagggactttttggctggaagacttccttgtattcctggtgaattaccaactctcaatgattgggaaaatcacttgacaactatatttcctgaggtcaggctgaagaggtatttggagatgagaggtgctgatggagggccttggagaagattgtgtgctttaccagcattttgggtagggttattgtacgatgaactttctctaaaaagtgttttggatatgacagctgattggactccagaagaaagacaaatgttaaggaataaggttcctgtaactggtctgaagacaccattccgagacggtttgctgaagcatgttgctgaagatgttctaaagttggcaaaggatggcttggagagaagaggcttcaaggaatcgggatttttgaatgaggttgccgaggtggttagaacaggtgtcactccagctgagaggcttttggaattgtatcatggaaagtgggagcaatccgtagatcatgtgtttgaggaattgctttattaaggtagtattgtctttcaaatgtctgtggaagattgtgtaatcctttggttatagttctggttgtctctcatttgagcttcatttagatataggaaataatataaatgtaatttttgccttgaggcttctcatatacttcgaa

SEQ ID NO: 42 - DNA, 1515 bp, Glycine max
atggctgtcgtttcgcgaagtgcgacgacctatacgcgccactacttaatacgacacgagtttgataggaaaacgaaaacctgcgttgccaataatagtttgtgttactctgctaagaaggctcctccaccgcagaggattgttggtggccgtagagtgattgttgctgcgagccctcccaccgaagacgctgtagttgccactgaccctctcacgaagcaggatctcgtcgattatcttgcctccggttgcaagcccaaggataaatggagaataggtactgaacatgagaagtttggttttgagattggaagcttgcgtcctatgaagtatgaccaaatagcagaattgctgaatggcattgctgagaggtttgactgggataaagtaatggaaggtgataaaattattggactcaaacaggggaagcagagcatatcattggagcctggtggtcagtttgaacttagtggagctcctcttgaaaccttgcatcagacttgtgctgaagttaattcccacctttatcaggttaaagctgttgctgaggaaatgggaattggatttttggggattggtttccagccaaagtggggaatcaaagacatacctataatgccaaagggaagatacgacatcatgaggaactacatgcctaaagttggctctcttgggcttgacatgatgttcaggacatgcactgtgcaggtcaatctggactttagttctgaagctgacatgatcaagaaatttcgtgcaggccttgctttgcagccgatagcaacggctctttttgcaaattcaccctttaaagagggaaagccaaatggttttgtcagtatgagaagccatatttggactgatactgataaggaccgcacaggcatgctgccttttgtttttgatgactcttttgggtttgagcaatatgttgattatgctcttgatgttcctatgtattttgtctatcggaaaaacagatatatcgactgcactggaaagaccttcagggactttttggctggaagacttccttgtattcctggtgaattaccaactctcaatgattgggaaaatcacttgacaactatatttcctgaggtcaggctgaagaggtatttggagatgagaggtgctgatggagggccttggagaagattgtgtgctttaccagcattttgggtagggttattgtacgatgaactttctctaaaaagtgttttggatatgacagctgattggactccagaagaaagacaaatgttaaggaataaggttcctgtaactggtctgaagacaccattccgagacggtttgctgaagcatgttgctgaagatgttctaaagttggcaaaggatggcttggagagaagaggcttcaaggaatcgggatttttgaatgaggttgccgaggtggttagaacaggtgtcactccagctgagaggcttttggaattgtatcatggaaagtgggagcaatccgtagatcatgtgtttgaggaattgctttattaa

SEQ ID NO: 43 - protein, 504 aa, Glycine max
MAVVSRSATTYTRHYLIRHEFDRKTKTCVANNSLCYSAKKAPPPQRIVGGRRVIVAASPPTEDAVVATDPLTKQDLVDYLASGCKPKDKWRIGTEHEKFGFEIGSLRPMKYDQIAELLNGIAERFDWDKVMEGDKIIGLKQGKQSISLEPGGQFELSGAPLETLHQTCAEVNSHLYQVKAVAEEMGIGFLGIGFQPKWGIKDIPIMPKGRYDIMRNYMPKVGSLGLDMMFRTCTVQVNLDFSSEADMIKKFRAGLALQPIATALFANSPFKEGKPNGFVSMRSHIWTDTDKDRTGMLPFVFDDSFGFEQYVDYALDVPMYFVYRKNRYIDCTGKTFRDFLAGRLPCIPGELPTLNDWENHLTTIFPEVRLKRYLEMRGADGGPWRRLCALPAFWVGLLYDELSLKSVLDMTADWTPEERQMLRNKVPVTGLKTPFRDGLLKHVAEDVLKLAKDGLERRGFKESGFLNEVAEVVRTGVTPAERLLELYHGKWEQSVDHVFEELLY

SEQ ID NO: 44 - DNA, 1356 bp, Glycine max
atggttgctgcgagccctcccaccgaagacgctgtagttgccactgaccctctcacgaagcaggatctcgtcgattatcttgcctccggttgcaagcccaaggataaatggagaataggtactgaacatgagaagtttggttttgagattggaagcttgcgtcctatgaagtatgaccaaatagcagaattgctgaatggcattgctgagaggtttgactgggataaagtaatggaaggtgataaaattattggactcaaacaggggaagcagagcatatcattggagcctggtggtcagtttgaacttagtggagctcctcttgaaaccttgcatcagacttgtgctgaagttaattcccacctttatcaggttaaagctgttgctgaggaaatgggaattggatttttggggattggtttccagccaaagtggggaatcaaagacatacctataatgccaaagggaagatacgacatcatgaggaactacatgcctaaagttggctctcttgggcttgacatgatgttcaggacatgcactgtgcaggtcaatctggactttagttctgaagctgacatgatcaagaaatttcgtgcaggccttgctttgcagccgatagcaacggctctttttgcaaattcaccctttaaagagggaaagccaaatggttttgtcagtatgagaagccatatttggactgatactgataaggaccgcacaggcatgctgccttttgtttttgatgactcttttgggtttgagcaatatgttgattatgctcttgatgttcctatgtattttgtctatcggaaaaacagatatatcgactgcactggaaagaccttcagggactttttggctggaagacttccttgtattcctggtgaattaccaactctcaatgattgggaaaatcacttgacaactatatttcctgaggtcaggctgaagaggtatttggagatgagaggtgctgatggagggccttggagaagattgtgtgctttaccagcattttgggtagggttattgtacgatgaactttctctaaaaagtgttttggatatgacagctgattggactccagaagaaagacaaatgttaaggaataaggttcctgtaactggtctgaagacaccattccgagacggtttgctgaagcatgttgctgaagatgttctaaagttggcaaaggatggcttggagagaagaggcttcaaggaatcgggatttttgaatgaggttgccgaggtggttagaacaggtgtcactccagctgagaggcttttggaattgtatcatggaaagtgggagcaatccgtagatcatgtgtttgaggaattgctttattaa

SEQ ID NO: 45 - protein, 451 aa, Glycine max
MVAASPPTEDAVVATDPLTKQDLVDYLASGCKPKDKWRIGTEHEKFGFEIGSLRPMKYDQIAELLNGIAERFDWDKVMEGDKIIGLKQGKQSISLEPGGQFELSGAPLETLHQTCAEVNSHLYQVKAVAEEMGIGFLGIGFQPKWGIKDIPIMPKGRYDIMRNYMPKVGSLGLDMMFRTCTVQVNLDFSSEADMIKKFRAGLALQPIATALFANSPFKEGKPNGFVSMRSHIWTDTDKDRTGMLPFVFDDSFGFEQYVDYALDVPMYFVYRKNRYIDCTGKTFRDFLAGRLPCIPGELPTLNDWENHLTTIFPEVRLKRYLEMRGADGGPWRRLCALPAFWVGLLYDELSLKSVLDMTADWTPEERQMLRNKVPVTGLKTPFRDGLLKHVAEDVLKLAKDGLERRGFKESGFLNEVAEVVRTGVTPAERLLELYHGKWEQSVDHVFEELLY

SEQ ID NO: 46 - DNA, 21 bp, Artificial Sequence
  primer PHN_GM-GSH2m
ccatggttgctgcgagccctc

SEQ ID NO: 47 - protein, 449 aa, Phaseolus vulgaris
MASPPTEDAVVATDPLTKQDLVDYLASGCKPREKWRIGTEHEKFGFEFGSLRPMKYEQIAELLNGIAERFDWDKIMEGDKIIGLKQGKQSISLEPGGQFELSGAPLETLHQTCAEVNSHLYQVKAVAEEMEIGFLGIGFQPKWGIEDIPVMPKGRYDIMRNYMPKVGSLGLDIMFRTCTVQVNLDFSSEADMIRKFRAGLALQPIATALFANSPFKEGKPNGFVSMRSHIWTDTDKDRTGMLPFVFDDSFGFEQYVDYALDVPMYFVYRKHRYIDCTGKTFRDFLAGRLPCIPGELPTLNDWENHLTTIFPEVRLKRYLEMRGADGGPWRRLCALPALWVGLLYDEASLQSLLDLTADWTPEERQMLRNKVPVTGLKTPFRDGLLKHVAEDVLQLAKDGLERRGFKESGFLNEVAEVVRTGVTPAERLLELYHGKWEQSVDHVFEELLY

SEQ ID NO: 48 - protein, 449 aa, Zinnia violacea
MASPPTEDAVVATEPLTKEDLVGYLASGCKPKENWRIGTEHEKFGFDLKTLRPMTYEQIAHLLNAISERFDWEKVMEGDNIIGLKQGKQSISLEPGGQFELSGAPLETLHQTCAEVNSHLYQVKAVAEEMGIGFIGIGFQPKWERKDIPIMPKGRYEIMRNYMPKVGSLGLDMMFRTCTVQVNLDFSSEADMIRKFRAGLALQPIATALFANSPFTEGKPNGYLSMRSQIWTDTDNDRSGMLPFVFDDSFGFEQYVEYALDVPMYFVYRKKKYIDCAGLSFRDFLAGKLPPIPGEYPTLNDWENHLTTIFPEVRLKRYLEMRGADGGPWRRLCALPAFWVGVLYDDISLQNVLDMTADWTQEERQMLRNKVPVAGLKTPFRDGLLKHVAEEVLKFAKDGLERRGYKETGFLNEVAEVVRTGLTPAEKLLELYHGKWGQSVDPVFEELLY

SEQ ID NO: 49 - protein, 450 aa, Glycine max
  misc_feature (44)..(44): Xaa can be any naturally occurring amino acid
MASPPTEDAVVATDPLTKQDLVDYLASGCKPKDKWRIGTEHEKXGFEIGSLRPMKYDQIAELLNGIAERFDWDKVMEGDKIIGLKQGKQSISLEPGGQFELSGAPLETLHQTCAEVNSHLYQVKAVAEEMGIGFLGIGFQPKWGIKDIPIMPKGRYDIMRNYMPKVGSLGLDMMFRTCTVQVNLDFSSEADMIKKFRAGLALQPIATALFANSPFKEGKPNGFVSMRSHIWTDTDKDRTGMLPFVFDDSFGFEQYVDYAXLDVPMYYVFRKHRYIDCTGKTFRDFLAGRLPCIPGELPTLNDWENHLTTIFALPAFRVELLNDEADGGPWRRLCALPAFWVGLLYDELSLKSVLDMTADWTPEERQMLRNKVPVTGLKTPFRDGLLKHVAEDVLKLAKDGLERRGFKESGFLNEVAEVVRTGVTPAERLLELYHGKWEQSVDHVFEELLY

SEQ ID NO: 50 - protein, 449 aa, Oryza sativa
MASPPTEEAVQMTEPLTKEDLVAYLVSGCKPKENWRIGTEHEKFGFEVDTLRPIKYDQIRDILNGLAERFDWDKIVEENNVIGLKQGKQSISLEPGGQFELSGAPLETLHQTCAEVNSHLYQVKAVGEEMGIGFLGIGFQPKWALSDIPIMPKGRYEIMRNYMPKVGSLGLDMMFRTCTVQVNLDFSSEQDMIRKFRTGLALQPIATAIFANSPFKEGKPNGYLSLRSHIWTDTDNNRSGMLPFVFDDSFGFERYVDYALDIPMYFVYRNKKYIDCTGMSFRDFMVGKLPQAPGELPTLNDWENHLTTIFPEVRLKRYLEMRGADGGPWRRLCALPVFWVGLLYDEESLQSISDMTSDWTNEEREMLRRKVPVTGLKTPFRDGYVRDLAEEILQLSKNGLERRGYKEVGFLREVDAVISSGVTPAERLLNLYETKWQRSVDPVFQELLY

(19) United States
(12) Patent Application Publication
(10) Pub. No.: US 2012/0004114 A1
(43) Pub. Date: Jan. 5, 2012

(54) NUCLEOTIDE SEQUENCES ENCODING GSH1 POLYPEPTIDES AND METHODS OF USE

(75) Inventors: Stephen M. Allen, Wilmington, DE (US); Jeffrey E. Habben, Urbandale, IA (US); Guofu Li, Johnston, IA (US); Emil M. Orozco, JR., Cochranville, PA (US); Hajime Sakai, Newark, DE (US); Nicholas J. Bate, Urbandale, IA (US); Carl R. Simmons, Des Moines, IA (US); Ken'Ichi Ogawa, Kyoto (JP)

(73) Assignee: E. I. DU PONT DE NEMOURS AND COMPANY AND PIONEER HI-BRED INTERNATIONAL, Wilmington, DE (US)

(21) Appl. No.: 13/141,102

(22) PCT Filed: Dec. 21, 2009
(86) PCT No.: PCT/US09/68906

§ 371 Date: Sep. 16, 2011

Related U.S. Application Data
(60) Provisional application No. 61/139,869, filed on Dec. 22, 2008.

(51) Int. Cl. C40B 20/08; C40B 20/00; A01H 5/00; A01H 5/10; C12N 15/52; C12N 15/63

(52) U.S. Cl. 506/6; 536/23.2; 435/320.1; 800/298; 800/320.1; 800/312; 800/322; 800/320; 800/306; 800/320.3; 800/314; 800/320.2; 506/2

(57) ABSTRACT

Isolated polynucleotides and polypeptides and recombinant DNA constructs useful for improving agronomic traits, compositions (such as plants or seeds) comprising these recombinant DNA constructs, and methods utilizing these recombinant DNA constructs. The recombinant DNA construct comprises a polynucleotide operably linked to a promoter that is functional in a plant, wherein said polynucleotide encodes a GSH1 polypeptide.

### CROSS REFERENCE TO RELATED APPLICATIONS

This application claims the benefit of U.S. Provisional Application No. 61/139,869, filed Dec. 22, 2008, the entire content of which is herein incorporated by reference.

### FIELD OF THE INVENTION

The field of invention relates to plant breeding and genetics and, in particular, relates to recombinant DNA constructs useful in plants for improvement of agronomic traits.

### BACKGROUND OF THE INVENTION

The enzyme glutamate-cysteine ligase (GSH1) catalyzes the first and rate-limiting step of glutathione biosynthesis. The GSH1 gene is encoded by a single-copy gene in Arabidopsis (locus At4g23100). The GSH1 polypeptide also has been called gamma-glutamylcysteine synthetase (γ-ECS), cadmium insensitive 2 (CAD2; Cobbett et al., 1998, Plant J. 16:73-78), phytoalexin deficient 2 (PAD2; Pansy et al., 2006, Plant J. 49:159-172) and root meristemless 1 (RML1; Vernoux et al., 2000, Plant Cell 12:97-109). The Arabidopsis GSH1 polypeptide has a transit peptide and is targeted to the plastid.

The GSH1 polypeptide is involved in the following biological processes: glutathione biosynthesis; response to heat; defense response to bacteria; incompatible interaction; glucosinolate biosynthetic process; indole phytoalexin biosynthetic process; flower development; response to jasmonic acid stimulus; response to cadmium ion; response to ozone; defense response to fungus and defense response to insects.

### SUMMARY OF THE INVENTION

The present invention includes:

In one embodiment, the present invention includes an isolated polynucleotide comprising: (a) a nucleotide sequence encoding a polypeptide with GSH1 activity, wherein the polypeptide has an amino acid sequence of at least 97%, 98%, 99% or 100% sequence identity, based on the Clustal V method of alignment with pairwise alignment default parameters of KTUPLE=1, GAP PENALTY=3, WINDOW=5 and DIAGONALS SAVED=5, when compared to SEQ ID NO:2, 4, 6, 12, 14, 16, 18, 30, 32, 43 or 45; or (b) the full complement of the nucleotide sequence of (a). The polynucleotide may comprise the amino acid sequence of SEQ ID NO:2, 4, 6, 12, 14, 16, 18, 30, 32, 43 or 45. The polynucleotide of may comprise SEQ ID NO:1, 3, 5, 11, 13, 15, 17, 29, 31, 42 or 44.

In another embodiment, the present invention includes a recombinant DNA construct comprising any of the isolated polynucleotides of the present invention operably linked to at least one regulatory sequence, and a transgenic cell, a transgenic plant and a transgenic seed, wherein each transgenic entity comprises the recombinant DNA construct.

In another embodiment, the present invention includes a plant comprising in its genome a recombinant DNA construct comprising a polynucleotide operably linked to at least one regulatory element, wherein said polynucleotide encodes a polypeptide having an amino acid sequence of at least 50% sequence identity, based on the Clustal V method of alignment, when compared to SEQ ID NO:2, 4, 6, 8, 10, 12, 14, 16, 18, 20, 22, 23, 24, 25, 26, 27, 28, 30, 32, 43, 45, 47, 48, 49 and 50, and wherein said plant exhibits an alteration of at least one agronomic characteristic when compared to a control plant not comprising said recombinant DNA construct.

In another embodiment, the present invention includes any plant of the current invention, wherein the plant exhibits an alteration of at least one agronomic characteristic when compared, under water limiting conditions, to a control plant not comprising said recombinant DNA construct.

In another embodiment, the present invention includes any plant of the current invention, wherein the plant exhibits an alteration of at least one agronomic characteristic when compared, under nitrogen limiting conditions, to a control plant not comprising said recombinant DNA construct.

In another embodiment, the present invention includes any plant of the current invention, wherein the plant exhibits an alteration of at least one agronomic characteristic when cultivated at a planting density higher than that which allows sufficient increases in biomass quantity per unit area and in seed yield per unit area for a control plant not comprising said recombinant DNA construct.

For any of the plants of the current invention, the at least one agronomic characteristic may be selected from the group consisting of greenness, yield, growth rate, biomass, fresh weight at maturation, dry weight at maturation, fruit yield, seed yield, total plant nitrogen content, fruit nitrogen content, seed nitrogen content, whole plant free amino acid content, fruit free amino acid content, seed free amino acid content, fruit protein content, seed protein content, protein content in a vegetative tissue, drought tolerance, nitrogen uptake, root lodging, harvest index, stalk lodging, plant height, ear height, ear length, early seedling vigor, seedling emergence under low temperature stress and disease resistance.

In another embodiment, the present invention includes any plant of the current invention wherein the plant exhibits an increase in seed yield, biomass, or both when compared to a control plant.

In another embodiment, the present invention includes any plant of the current invention wherein the plant comprises an alteration in root architecture when compared to a control plant.

In another embodiment, the present invention includes a seed that comprises in its genome a recombinant DNA construct comprising a polynucleotide operably linked to at least one regulatory element, wherein said polynucleotide encodes a polypeptide having an amino acid sequence of at least 50% sequence identity, based on the Clustal V method of alignment, when compared to SEQ ID NO:2, 4, 6, 8, 10, 12, 14, 16, 18, 20, 22, 23, 24, 25, 26, 27, 28, 30, 32, 43, 45, 47, 48, 49 and 50, and wherein a plant produced from said seed exhibits an increase in at least one trait selected from the group consisting of: drought tolerance, seed yield and biomass, when compared to a control plant not comprising said recombinant DNA construct.

In another embodiment, the present invention includes a method of determining an alteration of at least one agronomic characteristic in a plant, comprising: (a) obtaining a transgenic plant, wherein the transgenic plant comprises in its genome a recombinant DNA construct comprising a polynucleotide operably linked to at least one regulatory element, wherein said polynucleotide encodes a polypeptide having an amino acid sequence of at least 50% sequence identity, based on the Clustal V method of alignment, when compared to SEQ ID NO:2, 4, 6, 8, 10, 12, 14, 16, 18, 20, 22, 23, 24, 25, 26, 27, 28, 30, 32, 43, 45, 47, 48, 49 and 50: (b) obtaining a progeny plant derived from the transgenic plant, wherein the progeny plant comprises in its genome the recombinant DNA construct; and (c) determining whether the transgenic plant exhibits an alteration of at least one agronomic characteristic when compared to a control plant not comprising the recombinant DNA construct.

In another embodiment, the present invention includes any method of the current invention wherein said determining step also comprise determining whether the transgenic plant exhibits an alteration of at least one agronomic characteristic when compared, under water limiting conditions, to a control plant not comprising the recombinant DNA construct.

In another embodiment, the present invention includes any method of the current invention wherein said determining step also comprises determining whether the transgenic plant exhibits an alteration of at least one agronomic characteristic when compared, under nitrogen limiting conditions, to a control plant not comprising the recombinant DNA construct.

In another embodiment, the present invention includes any method of the current invention wherein said determining step also comprises determining whether the transgenic plant exhibits an alteration of at least one agronomic characteristic when cultivated at a planting density higher than that which allows sufficient increases in biomass quantity per unit area and in seed yield per unit area for a control plant not comprising the recombinant DNA construct.

In another embodiment, the present invention includes any method of the current invention wherein said at least one agronomic characteristic is at least one selected from the group consisting of greenness, yield, growth rate, biomass, fresh weight at maturation, dry weight at maturation, fruit yield, seed yield, total plant nitrogen content, fruit nitrogen content, seed nitrogen content, whole plant free amino acid content, fruit free amino acid content, seed free amino acid content, fruit protein content, seed protein content, protein content in a vegetative tissue, drought tolerance, nitrogen uptake, root lodging, harvest index, stalk lodging, plant height, ear height, ear length, early seedling vigor, seedling emergence under low temperature stress and disease resistance.

In another embodiment, the present invention includes any method of the current invention wherein said plant exhibits an increase in seed yield, biomass, or both when compared to said control plant.

In another embodiment, the present invention includes any method of the current invention wherein the plant further comprises and alteration in root architecture when compared to said control plant.

In another embodiment, the present invention includes any plant or seed of the current invention, or any method of the current invention, wherein the plant or seed of the composition or method is selected from the group consisting of: maize, soybean, sunflower, sorghum, canola, wheat, alfalfa, cotton, rice, barley, millet, sugar cane and switchgrass.

### BRIEF DESCRIPTION OF THE DRAWINGS AND SEQUENCE LISTING

The invention can be more fully understood from the following detailed description and the accompanying drawings and Sequence Listing which form a part of this application.

FIGS. 1A-1E present an alignment of the amino acid sequences of the GSH1 precursor polypeptides set forth in SEQ ID NOs:2, 8, 12, 30, 16, 20, 23, 25, 26, 27, 28 and the maize GSH1 polypeptide of SEQ ID NO:24 that lacks a transit peptide. A consensus sequence is presented where a residue is shown if identical in all sequences, otherwise, a period is shown.

FIG. 2 presents the percent sequence identities and divergence values for each pair of amino acid sequences presented in FIGS. 1A-1E.

FIGS. 3A-3C present an alignment of the amino acid sequences of the GSH1 mature polypeptides set forth in SEQ ID NOs:4, 10, 14, 32, 18, 22 and the maize GSH1 polypeptide of SEQ ID NO:24 that lacks a transit peptide. A consensus sequence is presented where a residue is shown if identical in all sequences, otherwise, a period is shown.

FIG. 4 presents the percent sequence identities and divergence values for each pair of amino acid sequences presented in FIGS. 3A-3C.

SEQ ID NO:1 is a nucleotide sequence encoding a soybean GSH1 precursor polypeptide and corresponds to a contig of the nucleotide sequences of the cDNA inserts of clones sr1.pk0076.f7 and sl2.pk0035.d12.

SEQ ID NO:2 is the amino acid sequence of the soybean GSH1 precursor polypeptide encoded SEQ ID NO:1.

SEQ ID NO:3 is a nucleotide sequence encoding a putative soybean GSH1 mature polypeptide, and corresponds to an ATG start codon followed by nucleotides 169-1515 of SEQ ID NO:1.

SEQ ID NO:4 is the amino acid sequence of the soybean GSH1 mature polypeptide encoded by SEQ ID NO:3.

SEQ ID NO:5 is a nucleotide sequence encoding a soybean GSH1 truncated polypeptide consisting of the carboxy-terminal 320 amino acids, and was prepared using the PCR primers of SEQ ID NO:37 and SEQ ID NO:38. The GSH1 gene fragment was amplified from cDNA generated from R6 pod tissue of the soybean variety JACK.

SEQ ID NO:6 is the amino acid sequence of the soybean GSH1 truncated polypeptide encoded by SEQ ID NO:5.

SEQ ID NO:7 is a nucleotide sequence encoding a maize GSH1 precursor polypeptide, designated Zm-GSH1a. SEQ ID NO:7 is a contig, designated PCO664734, assembled from 19 maize sequences.

SEQ ID NO:8 is the amino acid sequence of the Zm-GSH1a precursor polypeptide encoded by SEQ ID NO:7.

SEQ ID NO:9 is a nucleotide sequence encoding a putative Zm-GSH1a mature polypeptide, and corresponds to an ATG start codon followed by nucleotides 163-1350 of SEQ ID NO:7.

SEQ ID NO:10 is the amino acid sequence of the Zm-GSH1a mature polypeptide encoded by SEQ ID NO:9.

SEQ ID NO:11 is a nucleotide sequence encoding a second maize GSH1 precursor polypeptide, designated Zm-GSH1b. SEQ ID NO:11 is a contig, designated PCO664735, assembled from 44 maize sequences.

SEQ ID NO:12 is the amino acid sequence of the Zm-GSH1b precursor polypeptide encoded by SEQ ID NO:11.

SEQ ID NO:13 is a nucleotide sequence encoding a putative Zm-GSH1b mature polypeptide, and corresponds to an ATG start codon followed by nucleotides 157-1503 of SEQ ID NO:11.

SEQ ID NO:14 is the amino acid sequence of the Zm-GSH1b mature polypeptide encoded by SEQ ID NO:13.

SEQ ID NO:15 is a nucleotide sequence encoding a sunflower GSH1 precursor polypeptide and corresponds to a contig of the nucleotide sequences of the cDNA inserts of clones hss1c.pk021.l4, hls1c.pk008.e8, hso1c.pk021.k15 and the EST sequence of NCBI GI No. 22468001.

SEQ ID NO:16 is the amino acid sequence of the sunflower GSH1 precursor polypeptide encoded by SEQ ID NO:15.

SEQ ID NO:17 is a nucleotide sequence encoding a putative sunflower mature polypeptide, and corresponds to an ATG start codon followed by nucleotides 251-1603 of SEQ ID NO:15.

SEQ ID NO:18 is the amino acid sequence of the sunflower GSH1 mature polypeptide encoded by SEQ ID NO:17.

SEQ ID NO:19 is the nucleotide sequence corresponding to NCBI GI No. 1742962, for a cDNA encoding an Arabidopsis GSH1 precursor polypeptide.

SEQ ID NO:20 is the amino acid sequence of the Arabidopsis GSH1 precursor polypeptide encoded by SEQ ID NO:19.

SEQ ID NO:21 is the nucleotide sequence from done custom7.pk139.f7 encoding an ATG start codon followed by a sequence encoding the mature Arabidopsis GSH1 polypeptide.

SEQ ID NO:22 is the amino acid sequence of the mature Arabidopsis GSH1 polypeptide encoded by SEQ ID NO:21.

SEQ ID NO:23 is the amino acid sequence corresponding to NCBI GI No. 6651029 for a Phaseoius vulgaris GSH1 precursor polypeptide.

SEQ ID NO:24 is the amino acid sequence corresponding to NCBI GI No. 162464176 for a Zea mays GSH1 polypeptide. This amino acid sequence does not contain a chloroplast transit peptide.

SEQ ID NO:25 is the amino acid sequence corresponding to NCBI GI No. 50058088 for a Zinnia violacea GSH1 precursor polypeptide.

SEQ ID NO:26 is the amino acid sequence presented as SEQ ID NO: 252666 of US Patent Publication No. US2004031072-A1 for a soybean GSH1 precursor polypeptide.

SEQ ID NO:27 is the amino acid sequence presented as SEQ ID NO: 56195 of Japanese Patent Publication No. JP2005185101-A for a rice GSH1 precursor polypeptide.

SEQ ID NO:28 is the amino acid sequence presented as SEQ ID NO: 2265 of International POT Patent Publication No. WO2002010210-A2 for an Arabidopsis GSH1 precursor polypeptide.

SEQ ID NO:29 is a nucleotide sequence encoding a maize GSH1 precursor polypeptide, designated Zm-GSH1c, derived from genomic sequencing of a region of chromosome 6.

SEQ ID NO:30 is the amino acid sequence of the Zm-GSH1c precursor polypeptide encoded by SEQ ID NO:29.

SEQ ID NO:31 is a nucleotide sequence encoding a putative Zm-GSH1c mature polypeptide, and corresponds to an ATG start codon followed by nucleotides 166-1512 of SEQ ID NO:29.

SEQ ID NO:32 is the amino acid sequence of the Zm-GSH1c mature polypeptide encoded by SEQ ID NO:31.

SEQ ID NO:33 is the nucleotide sequence of the attB1 site.

SEQ ID NO:34 is the nucleotide sequence of the attB2 site.

SEQ ID NO:35 is the nucleotide sequence of the VC062 primer, containing the T3 promoter and attB1 site, useful to amplify cDNA inserts cloned into a BLUESCRIPT® II SK(+) vector (Stratagene).

SEQ ID NO:36 is the nucleotide sequence of the VC063 primer, containing the T7 promoter and attB2 site, useful to amplify cDNA inserts cloned into a BLUESCRIPT® II SK(+) vector (Stratagene).

SEQ ID NO:37 is the forward primer, “GM-GSH-F3”, used to PCR amplify the nucleic acid sequence of SEQ ID NO:5 encoding the soybean truncated GSH1 polypeptide. This primer has an Ncol site at the 5′ end.

SEQ ID NO:38 is the reverse primer, “GM-GSH-R1”, used to PCR amplify the nucleic acid sequence of SEQ ID NO:5 encoding the soybean truncated GSH1 polypeptide. This primer has an Sful site at the 5′ end.

SEQ ID NO:39 is the forward primer, “PHN_{—}131845”, used to FOR amplify the nucleic acid sequence of SEQ ID NO:41 encoding the soybean precursor GM-GSH1b polypeptide from cDNA clone ssl.pk0035.b9. This primer has an Ncol site next to the first nucleotide at the 5′ end.

SEQ ID NO:40 is the reverse primer, “PHN_{—}131846”, used to FOR amplify the nucleic acid sequence of SEQ ID NO:41 encoding the soybean precursor GM-GSH1b polypeptide from cDNA clone ssl.pk0035.b9. This primer has an Sful site at the 5′ end.

SEQ ID NO:41 is the nucleotide sequence of the FOR product obtained from cDNA clone ssl.pk0035.b9; it encodes the GM-GSH1b precursor polypeptide.

SEQ ID NO:42 is the nucleotide sequence of the protein-coding locus from cDNA clone ssl.pk0035.b9; it encodes the GM-GSH1b precursor polypeptide.

SEQ ID NO:43 is the amino acid sequence of the soybean GM-GSH1b precursor polypeptide encoded by SEQ ID NO:42.

SEQ ID NO:44 is the nucleotide sequence of a putative GM-GSH1b mature polypeptide, and corresponds to an ATG start codon followed by nucleotides 163-1515 of SEQ ID NO:42.

SEQ ID NO:45 is the amino acid sequence of the putative GM-GSH1b mature polypeptide encoded by SEQ ID NO:44.

SEQ ID NO:46 is the nucleotide sequence of forward primer PHN_GM-GSH2m, used with SEQ ID NO:40 to PCR amplify SEQ ID NO:44, the sequence encoding the putative GM-GSH1b mature polypeptide.

SEQ ID NO:47 is the amino acid sequence of a putative mature GSH1 polypeptide from Phaseolus vulgaris; it corresponds to SEQ ID NO:23 with a deletion of amino acid residues 2-60 containing the transit peptide.

SEQ ID NO:48 is the amino acid sequence of a putative mature GSH1 polypeptide from Zinnia violacea; it corresponds to SEQ ID NO:25 with a deletion of amino acid residues 2-75 containing the transit peptide.

SEQ ID NO:49 is the amino acid sequence of a putative mature GSH1 polypeptide from Glycine max; it corresponds to SEQ ID NO:26 with a deletion of amino acid residues 2-56 containing the transit peptide.

SEQ ID NO:50 is the amino acid sequence of a putative mature GSH1 polypeptide from Oryza sativa; it corresponds to SEQ ID NO:27 with a deletion of amino acid residues 2-44 containing the transit peptide.

The sequence descriptions and Sequence Listing attached hereto comply with the rules governing nucleotide and/or amino acid sequence disclosures in patent applications as set forth in 37 C.F.R. §1.821-1.825.

The Sequence Listing contains the one letter code for nucleotide sequence characters and the three letter codes for amino acids as defined in conformity with the IUPAC-IUBMB standards described in Nucleic Acids Res. 13:3021-3030 (1985) and in the Biochemical J. 219 (No. 2):345-373 (1984) which are herein incorporated by reference. The symbols and format used for nucleotide and amino acid sequence data comply with the rules set forth in 37 C.F.R. §1.822.

### DETAILED DESCRIPTION

The disclosure of each reference set forth herein is hereby incorporated by reference in its entirety.

As used herein and in the appended claims, the singular forms “a”, “an”, and “the” include plural reference unless the context clearly dictates otherwise. Thus, for example, reference to “a plant” includes a plurality of such plants, reference to “a cell” includes one or more cells and equivalents thereof known to those skilled in the art, and so forth.

As used herein:

The enzyme glutamate-cysteine ligase (GSH1; EC 6.3.2.2), which catalyzes the first and rate-limiting step of glutathione biosynthesis, is also known as gamma-glutamylcysteine synthetase, (γ-ECS), cadmium insensitive 2 (CAD2), phytoalexin deficient 2 (PAD2) and root meristemless 1 (RML1).

A polypeptide with “GSH1 activity” is a polypeptide with glutamate-cysteine ligase activity or gamma-glutamylcysteine synthetase activity (EC 6.3.2.2). Enzymatic assays are available for determining GSH1 activity (Noctor and Foyer, 1998, Anal. Biochem. 264:98-110; Noctor et al., 2002, Exp. Bot. 53:1283-1304; Hothorn et al., 2006, J. Biol. Chem. 281:27557-27565).

A transformed plant having a glutamate-cysteine ligase (GSH1) gene has been found to be increased in at least one agronomic trait selected from the group consisting of the number of flowers, the number of seeds, and the weight of seeds, as compared to a corresponding wild-type plant, when cultivated at a planting density higher than that which allows sufficient increases in biomass quantity per unit area and in seed yield per unit area. (EP2123753A1).

The “planting density” means the number of individuals planted per unit area. Generally, in a case where plants are grown, seedlings or young plants are planted or thinned at appropriate intervals. This is because when a planting density for individuals increases, the biomass productivity per individual decreases and the biomass productivity per unit area levels off. As such, each plant has a planting density appropriate for its biomass productivity per unit area. Planting of the plant at a planting density higher than the appropriate planting density causes a decrease in crop yields with respect to purchases costs of seeds or seedlings, and therefore such planting is not preferable. In the present invention, the “planting density which allows sufficient increases in biomass quantity per unit area and in seed yield per unit area” means an optimal planting density for each breed (that is, an optimal planting density at which the biomass productivity per unit area is largest). Although the optimal planting density varies depending on the breed of plant, a person skilled in the art can easily know an optimal planting density for each plant to be used. Even in a case where the plant according to the present invention is cultivated at a planting density higher than that which allows sufficient increases in biomass quantity per unit area and in seed yield per unit area, the biomass quantity per unit area or the seed yield per unit area is further increased in comparison with that of a parent plant/wild-type plant. The planting density at which the plant of the present invention is cultivated is not limited to one higher than the optimal planting density. The planting density is preferably not less than 30%, more preferably not less than 60%, further preferably not less than 100% of the optimal planting density for each breed.

In the present invention, the “expression level of GSH1” means an amount of GSH1 mRNA or an amount of GSH1 protein.

The “increase in an expression level of GSH1” means that a plant is increased in the mRNA level or the protein level in comparison with an expression level of GSH1 of a parent plant of the same breed. The expression level of GSH1 is compared with that of GSH1 at a corresponding part in the parent plant of the same breed cultured under the same condition. A case where the expression level increases at least 1.1 times greater than that of the parent plant is preferably considered as a case where the expression level is increased. Here, it is more preferable that the expression level of the plant has a significant difference of 5% by a t-test compared with that of the parent plant, in order to be considered that there is an increase in the expression level. It is preferable that the expression levels of the plant and the parent plant be measured at the same time by the same method. However, data stored as background data may be also used.

In the present invention, “the number of flowers” means the number of flowers of a single individual or plants planted per unit area.

Further, “the number of seeds” means the number of seeds of a single individual or plants planted per unit area.

The “increase in the number of flowers” means that a plant increases in the number of flowers in comparison with that of a parent plant of the same breed cultivated under the same condition. Further, the “increase in the number of seeds” means that the plant increases in the number of seeds in comparison with that of a parent plant of the same breed cultivated under the same condition.

In the present specification, the “GSH1 having no chloroplast targeting signal peptide” means a GSH1 having no chloroplast targeting signal peptide that functions properly. The GSH1 having no chloroplast targeting signal peptide encompasses: one that lacks an entire chloroplast targeting signal peptide region that is normally present; one that partially lacks a chloroplast targeting signal peptide region and lost of the chloroplast targeting function; one that lost a chloroplast targeting function due to substitution or addition of amino acids; one that normally has no chloroplast targeting signal peptide; and the like.

Here, the expression “one or several amino acids are deleted, substituted, or added” means that an amino acid(s) is/are deleted, substituted, or added to the extent that the amino acid(s) (preferably not more than 10, more preferably not more than 7, further preferably not more than 5 amino acids) are deleted, substituted, or added from/in/to the amino acid sequence by a well-known peptide mutant production method such as a site-directed mutagenesis method. Such a protein mutant obtained in the above manner is not limited to an artificially-mutated protein mutant produced by the well-known polypeptide mutant production method, but may be a naturally-occurred protein mutant obtained by isolating it from among natural proteins.

It has been well known in the related field of the present invention that several amino acids in an amino sequence of a protein can be easily modified without significantly affecting the structure or function of the protein. Further, it has been also well known that some natural proteins have mutants that do not significantly change the structures or functions of these natural proteins.

Preferable mutants have conservative or nonconservative substitution, deletion, or addition of amino acids. Silent substitution, addition, and deletion are preferred, and conservative substitution is especially preferred. These mutations do not change polypeptide activity of the present invention.

Typical conservative substitutions encompass: substitution of one of aliphatic amino acids Ala, Val, Leu, and Ile with another amino acid; exchange of hydroxyl residues Ser and Thr; exchange of acidic residues Asp and Glu; substitution between amide residues Asn and Gln; exchange of basic residues Lys and Arg; and substitution between aromatic residues Phe and Tyr.

In the present invention, a polynucleotide that hybridizes under a stringent condition with the polynucleotide of the current invention can be used, as long as the polynucleotide can encode a protein having the GSH1 activity. Such a polynucleotide encompass, for example, a polynucleotide encoding a polypeptide having an amino acid sequence in which one or several amino acids are deleted, substituted, or added from/in/to the amino acid sequence of any of the polypeptides of the current invention.

In the present invention, the “stringent condition” means that hybridization occurs only when sequences share at least 90%, preferably at least 95%, most preferably at least 97% similarity with each other. More specifically, the stringent condition may be a condition where polynucleotides are incubated in a hybridization solution (50% formamide, 5×SSC [150 mM NaCl, 15 mM trisodium citrate], 50 mM sodium phosphate [pH 7.6], 5×Denhart's solution, 10% dextran sulfate, and 20 μg/ml of sheared denatured salmon sperm DNA) overnight at 42° C., and then the filter is washed with 0.1×SSC at about 65° C.

The hybridization can be carried out by well-known methods such as a method disclosed in Sambrook at al., Molecular Cloning, A Laboratory Manual, 3rd Ed., Cold Spring Harbor Laboratory (2001). Normally, stringency increases (hybridization becomes difficult) at a higher temperature and at a lower salt concentration. As stringency increases more, a more homologous polynucleotide can be obtained.

In the present specification, the “biomass quantity” means the dry weight of an individual plant. Further, the “seed yield” means the weight of all seeds of a single individual plant or seed yield per unit area.

In the present invention, the “harvest index” means a value calculated by dividing “the weight of all seeds of an individual plant” by “the dry weight of the individual plant including the seed weight”,

“Arabidopsis” and “Arabidopsis thaliana” are used interchangeably herein, unless otherwise indicated.

The terms “monocot” and “monocotyledonous plant” are used interchangeably herein. A monocot of the current invention includes the Gramineae.

The terms “dicot” and “dicotyledonous plant” are used interchangeably herein. A dicot of the current invention includes the following families: Brassicaceae, Leguminosae, and Solanaceae.

The terms “full complement” and “full-length complement” are used interchangeably herein, and refer to a complement of a given nucleotide sequence, wherein the complement and the nucleotide sequence consist of the same number of nucleotides and are 100% complementary.

An “Expressed Sequence Tag” (“EST”) is a DNA sequence derived from a cDNA library and therefore is a sequence which has been transcribed. An EST is typically obtained by a single sequencing pass of a cDNA insert. The sequence of an entire cDNA insert is termed the “Full-Insert Sequence” (“FIS”). A “Contig” sequence is a sequence assembled from two or more sequences that can be selected from, but not limited to, the group consisting of an EST, FIS and PCR sequence. A sequence encoding an entire or functional protein is termed a “Complete Gene Sequence” (“CGS”) and can be derived from an FIS or a contig.

A “trait” refers to a physiological, morphological, biochemical, or physical characteristic of a plant or particular plant material or cell. In some instances, this characteristic is visible to the human eye, such as seed or plant size, or can be measured by biochemical techniques, such as detecting the protein, starch, or oil content of seed or leaves, or by observation of a metabolic or physiological process, e.g. by measuring tolerance to water deprivation or particular salt or sugar concentrations, or by the observation of the expression level of a gene or genes, or by agricultural observations such as osmotic stress tolerance or yield.

“Agronomic characteristic” is a measurable parameter including but not limited to, greenness, yield, growth rate, biomass, fresh weight at maturation, dry weight at maturation, fruit yield, seed yield, total plant nitrogen content, fruit nitrogen content, seed nitrogen content, nitrogen content in a vegetative tissue, total plant free amino acid content, fruit free amino acid content, seed free amino acid content, free amino acid content in a vegetative tissue, total plant protein content, fruit protein content, seed protein content, protein content in a vegetative tissue, drought tolerance, nitrogen uptake, root lodging, harvest index, stalk lodging, plant height, ear height, ear length, salt tolerance, early seedling vigor and seedling emergence under low temperature stress.

Increased biomass can be measured, for example, as an increase in plant height, plant total leaf area, plant fresh weight, plant dry weight or plant seed yield, as compared with control plants.

The ability to increase the biomass or size of a plant would have several important commercial applications. Crop species may be generated that produce larger cultivars, generating higher yield in, for example, plants in which the vegetative portion of the plant is useful as food, biofuel or both.

Increased leaf size may be of particular interest. Increasing leaf biomass can be used to increase production of plant-derived pharmaceutical or industrial products. An increase in total plant photosynthesis is typically achieved by increasing leaf area of the plant. Additional photosynthetic capacity may be used to increase the yield derived from particular plant tissue, including the leaves, roots, fruits or seed, or permit the growth of a plant under decreased light intensity or under high light intensity.

Modification of the biomass of another tissue, such as root tissue, may be useful to improve a plant's ability to grow under harsh environmental conditions, including drought or nutrient deprivation, because larger roots may better reach water or nutrients or take up water or nutrients.

For some ornamental plants, the ability to provide larger varieties would be highly desirable. For many plants, including fruit-bearing trees, trees that are used for lumber production, or trees and shrubs that serve as view or wind screens, increased stature provides improved benefits in the forms of greater yield or improved screening.

“Transgenic” refers to any cell, cell line, callus, tissue, plant part or plant, the genome of which has been altered by the presence of a heterologous nucleic acid, such as a recombinant DNA construct, including those initial transgenic events as well as those created by sexual crosses or asexual propagation from the initial transgenic event. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation.

“Genome” as it applies to plant cells encompasses not only chromosomal DNA found within the nucleus, but organelle DNA found within subcellular components (e.g., mitochondrial, plastid) of the cell.

“Plant” includes reference to whole plants, plant organs, plant tissues, seeds and plant cells and progeny of same. Plant cells include, without limitation, cells from seeds, suspension cultures, embryos, meristematic regions, callus tissue, leaves, roots, shoots, gametophytes, sporophytes, pollen, and microspores.

“Progeny” comprises any subsequent generation of a plant.

“Transgenic plant” includes reference to a plant which comprises within its genome a heterologous polynucleotide. The heterologous polynucleotide may be stably integrated within the genome such that the polynucleotide is passed on to successive generations. The heterologous polynucleotide may be integrated into the genome alone or as part of a recombinant DNA construct.

“Heterologous” with respect to sequence means a sequence that originates from a foreign species, or, if from the same species, is substantially modified from its native form in composition and/or genomic locus by deliberate human intervention.

“Polynucleotide”, “nucleic acid sequence”, “nucleotide sequence”, or “nucleic acid fragment” are used interchangeably and is a polymer of RNA or DNA that is single- or double-stranded, optionally containing synthetic, non-natural or altered nucleotide bases. Nucleotides (usually found in their 5′-monophosphate form) are referred to by their single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

“Polypeptide”, “peptide”, “amino acid sequence” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The terms “polypeptide”, “peptide”, “amino acid sequence”, and “protein” are also inclusive of modifications including, but not limited to, glycosylation, lipid attachment, gamma-carboxylation of glutamic add residues, hydroxylation and ADP-ribosylation.

“Messenger RNA (mRNA)” refers to the RNA that is without introns and that can be translated into protein by the cell.

“cDNA” refers to a DNA that is complementary to and synthesized from a mRNA template using the enzyme reverse transcriptase. The cDNA can be single-stranded or converted into the double-stranded form using the Klenow fragment of DNA polymerase I.

“Mature” protein refers to a post-translationally processed polypeptide; i.e., one from which any pre- or pro-peptides present in the primary translation product have been removed.

“Precursor” protein refers to the primary product of translation of mRNA; i.e., with pre- and pro-peptides still present. Pre- and pro-peptides may be and are not limited to intracellular localization signals.

“Isolated” refers to materials, such as nucleic acid molecules and/or proteins, which are substantially free or otherwise removed from components that normally accompany or interact with the materials in a naturally occurring environment. Isolated polynucleotides may be purified from a host cell in which they naturally occur. Conventional nucleic acid purification methods known to skilled artisans may be used to obtain isolated polynucleotides. The term also embraces recombinant polynucleotides and chemically synthesized polynucleolides.

“Recombinant” refers to an artificial combination of two otherwise separated segments of sequence, e.g., by chemical synthesis or by the manipulation of isolated segments of nucleic acids by genetic engineering techniques. “Recombinant” also includes reference to a cell or vector, that has been modified by the introduction of a heterologous nucleic acid or a cell derived from a cell so modified, but does not encompass the alteration of the cell or vector by naturally occurring events (e.g., spontaneous mutation, natural transformation/transduction/transposition) such as those occurring without deliberate human intervention.

“Recombinant DNA construct” refers to a combination of nucleic acid fragments that are not normally found together in nature. Accordingly, a recombinant DNA construct may comprise regulatory sequences and coding sequences that are derived from different sources, or regulatory sequences and coding sequences derived from the same source, but arranged in a manner different than that normally found in nature.

The terms “entry clone” and “entry vector” are used interchangeably herein.

“Regulatory sequences” refer to nucleotide sequences located upstream (5′ non-coding sequences), within, or downstream (3′ non-coding sequences) of a coding sequence, and which influence the transcription. RNA processing or stability, or translation of the associated coding sequence. Regulatory sequences may include, but are not limited to, promoters, translation leader sequences, introns, and polyadenylation recognition sequences. The terms “regulatory sequence” and “regulatory element” are used interchangeably herein.

“Promoter” refers to a nucleic acid fragment capable of controlling transcription of another nucleic acid fragment.

“Promoter functional in a plant” is a promoter capable of controlling transcription in plant cells whether or not its origin is from a plant cell.

“Tissue-specific promoter” and “tissue-preferred promoter” are used interchangeably, and refer to a promoter that is expressed predominantly but not necessarily exclusively in one tissue or organ, but that may also be expressed in one specific cell.

“Developmentally regulated promoter” refers to a promoter whose activity is determined by developmental events.

“Operably linked” refers to the association of nucleic acid fragments in a single fragment so that the function of one is regulated by the other. For example, a promoter is operably linked with a nucleic acid fragment when it is capable of regulating the transcription of that nucleic acid fragment.

“Expression” refers to the production of a functional product. For example, expression of a nucleic acid fragment may refer to transcription of the nucleic acid fragment (e.g., transcription resulting in mRNA or functional RNA) and/or translation of mRNA into a precursor or mature protein.

“Phenotype” means the detectable characteristics of a cell or organism.

“Introduced” in the context of inserting a nucleic acid fragment (e.g., a recombinant DNA construct) into a cell, means “transfection” or “transformation” or “transduction” and includes reference to the incorporation of a nucleic acid fragment into a eukaryotic or prokaryotic cell where the nucleic acid fragment may be incorporated into the genome of the cell (e.g., chromosome, plasmid, plastid or mitochondrial DNA), converted into an autonomous replicon, or transiently expressed (e.g., transfected mRNA).

A “transformed cell” is any cell into which a nucleic acid fragment (e.g., a recombinant DNA construct) has been introduced.

“Transformation” as used herein refers to both stable transformation and transient transformation.

“Stable transformation” refers to the introduction of a nucleic acid fragment into a genome of a host organism resulting in genetically stable inheritance. Once stably transformed, the nucleic acid fragment is stably integrated in the genome of the host organism and any subsequent generation.

“Transient transformation” refers to the introduction of a nucleic acid fragment into the nucleus, or DNA-containing organelle, of a host organism resulting in gene expression without genetically stable inheritance.

“Allele” is one of several alternative forms of a gene occupying a given locus on a chromosome. When the alleles present at a given locus on a pair of homologous chromosomes in a diploid plant are the same that plant is homozygous at that locus. If the alleles present at a given locus on a pair of homologous chromosomes in a diploid plant differ that plant is heterozygous at that locus. If a transgene is present on one of a pair of homologous chromosomes in a diploid plant that plant is hemizygous at that locus.

A “chloroplast transit peptide” is an amino acid sequence which is translated in conjunction with a protein and directs the protein to the chloroplast or other plastid types present in the cell in which the protein is made. “Chloroplast transit sequence” refers to a nucleotide sequence that encodes a chloroplast transit peptide. A “signal peptide” is an amino acid sequence which is translated in conjunction with a protein and directs the protein to the secretory system (Chrispeels (1991)Ann. Rev. Plant Phys. Plant Mol. Biol. 42:21-53). If the protein is to be directed to a vacuole, a vacuolar targeting signal (supra) can further be added, or if to the endoplasmic reticulum, endoplasmic reticulum retention signal (supra) may be added. If the protein is to be directed to the nucleus, any signal peptide present should be removed and instead a nuclear localization signal included (Raikhel (1992) Plant Phys. 100:1627-1632). A “mitochondrial signal peptide” is an amino add sequence which directs a precursor protein into the mitochondria (Zhang and Glaser (2002) Trends Plant Sci 7:14-21).

Sequence alignments and percent identity calculations may be determined using a variety of comparison methods designed to detect homologous sequences including, but not limited to, the MEGALIGN® program of the LASERGENE® bioinformatics computing suite (DNASTAR® Inc., Madison, Wis.). Unless stated otherwise, multiple alignment of the sequences provided herein were performed using the Clustal V method of alignment (Higgins and Sharp (1989) CABIOS. 5:151-153) with the default parameters (GAP PENALTY=10, GAP LENGTH PENALTY=10). Default parameters for pairwise alignments and calculation of percent identity of protein sequences using the Clustal V method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4. After alignment of the sequences, using the Clustal V program, it is possible to obtain “percent identity” and “divergence” values by viewing the “sequence distances” table on the same program; unless stated otherwise, percent identities and divergences provided and claimed herein were calculated in this manner.

Standard recombinant DNA and molecular cloning techniques used herein are well known in the art and are described more fully in Sambrook, J., Fritsch, E. F. and Maniatis, T. Molecular Cloning: A Laboratory Manual; Cold Spring Harbor Laboratory Press Cold Spring Harbor, 1989 (hereinafter “Sambrook”).

Turning now to embodiments:

Embodiments include isolated polynucleotides and polypeptides, recombinant DNA constructs useful for conferring drought tolerance, compositions (such as plants or seeds) comprising these recombinant DNA constructs, and methods utilizing these recombinant DNA constructs.

Isolated Polynucleotides and Polypeptides:

The present invention includes the following isolated polynucleotides and polypeptides:

An isolated polynucleotide comprising: (i) a nucleic acid sequence encoding a polypeptide having an amino add sequence of at least 50%, 51%, 52%, 53%, 54%, 55%, 56%, 57%, 58%, 59%, 60%, 56%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO:2, 4, 6, 12, 14, 16, 18, 30, 32, 43 or 45; or (ii) a full complement of the nucleic add sequence of (i), wherein the full complement and the nucleic add sequence of (i) consist of the same number of nucleotides and are 100% complementary. Any of the foregoing isolated polynucleotides may be utilized in any recombinant DNA constructs (including suppression DNA constructs) of the present invention. The polypeptide is preferably a GSH1 polypeptide.

An isolated polypeptide having an amino acid sequence of at least 50%, 51%, 52%, 53%, 54%, 55%, 56%, 57%, 58%, 59%, 60%, 56%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO:2, 4, 6, 12, 14, 16, 18, 30, 32, 43 or 45. The polypeptide is preferably a GSH1 polypeptide.

An isolated polynucleotide comprising (i) a nucleic acid sequence of at least 50%, 51%, 52%, 53%, 54%, 55%, 56%, 57%, 58%, 59%, 60%, 56%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO:1, 3, 5, 11, 13, 15, 17, 29, 31, 42 or 44; or (ii) a full complement of the nucleic acid sequence of (i). Any of the foregoing isolated polynucleotides may be utilized in any recombinant DNA constructs (including suppression DNA constructs) of the present invention. The isolated polynucleotide preferably encodes a GSH1 polypeptide.

Recombinant DNA Constructs and Suppression DNA Constructs:

In one aspect, the present invention includes recombinant DNA constructs (including suppression DNA constructs).

In one embodiment, a recombinant DNA construct comprises a polynucleotide operably linked to at least one regulatory sequence (e.g., a promoter functional in a plant), wherein the polynucleotide comprises (i) a nucleic acid sequence encoding an amino acid sequence of at least 50%, 51%, 52%, 53%, 54%, 55%, 56%, 57%, 58%, 59%, 60%, 56%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO:2, 4, 6, 8, 10, 12, 14, 16, 18, 20, 22, 23, 24, 25, 26, 27, 28, 30, 32, 43, 45, 47, 48, 49 and 50; or (ii) a full complement of the nucleic acid sequence of (i).

In another embodiment, a recombinant DNA construct comprises a polynucleotide operably linked to at least one regulatory sequence (e.g., a promoter functional in a plant), wherein said polynucleotide comprises (i) a nucleic acid sequence of at least 50%, 51%, 52%, 53%, 54%, 55%, 56%, 57%, 58%, 59%, 60%, 56%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO:1, 3, 5, 7, 9, 11, 13, 15, 17, 19, 21, 29 or 31; or (ii) a full complement of the nucleic acid sequence of (i).

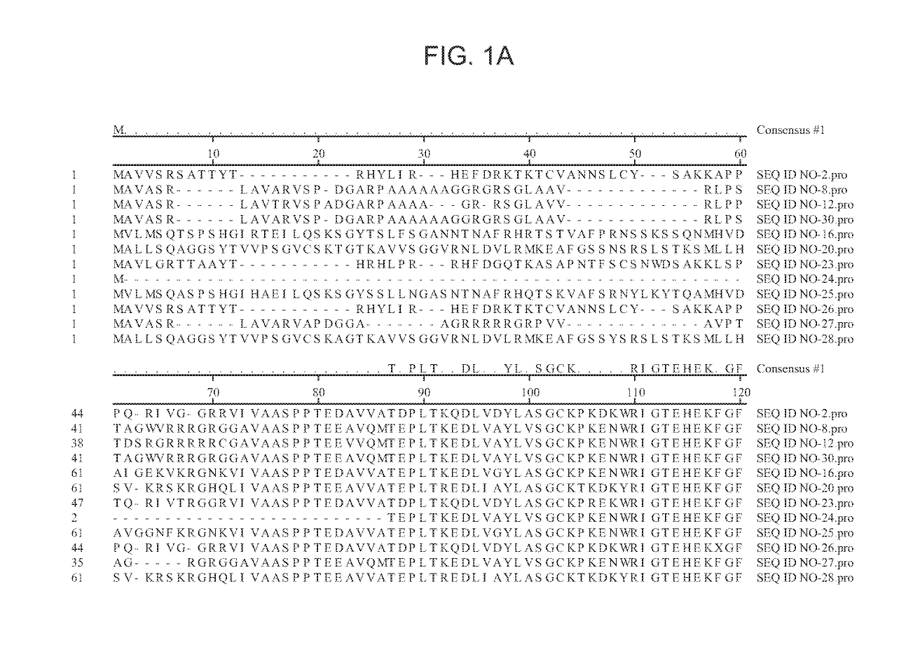

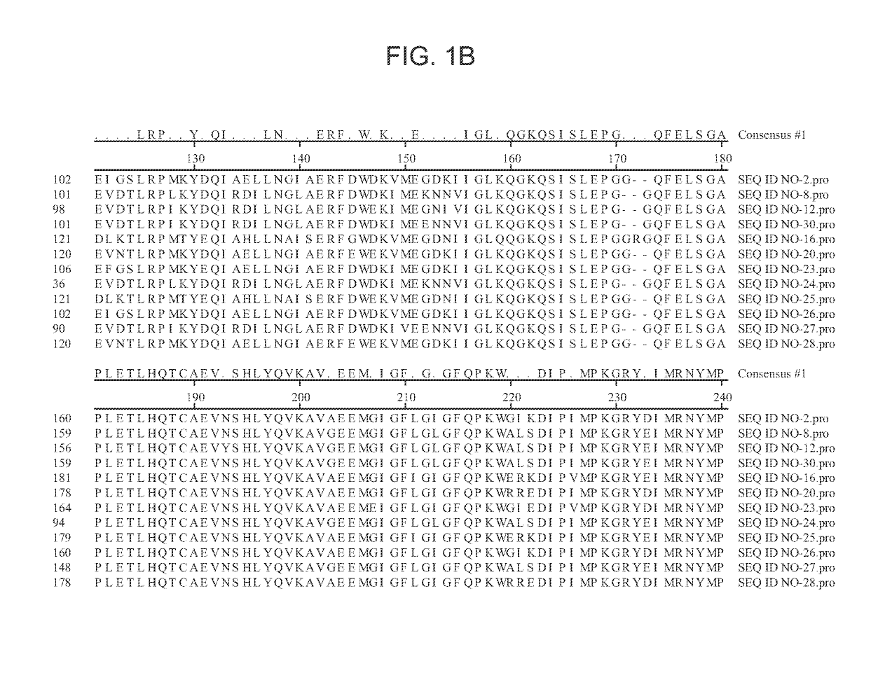

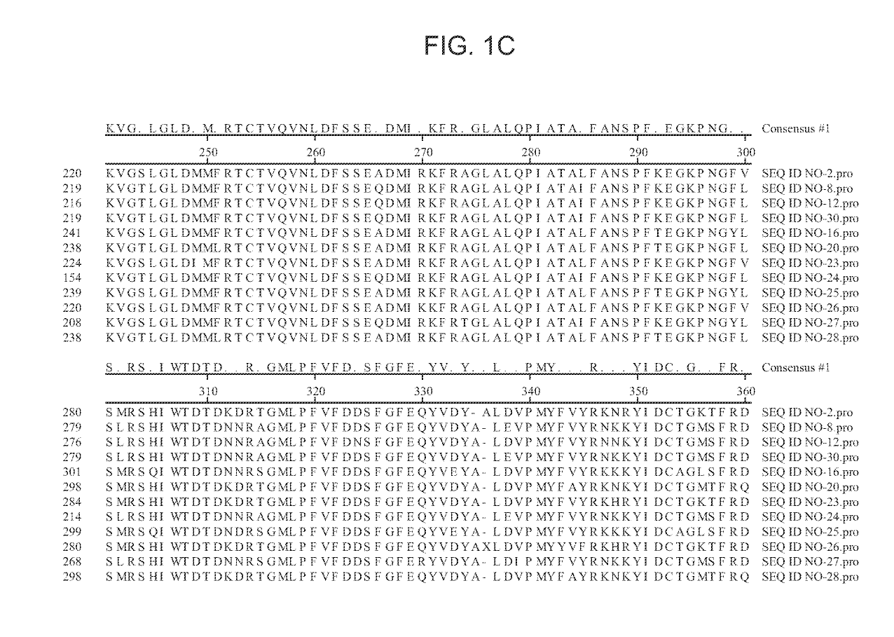

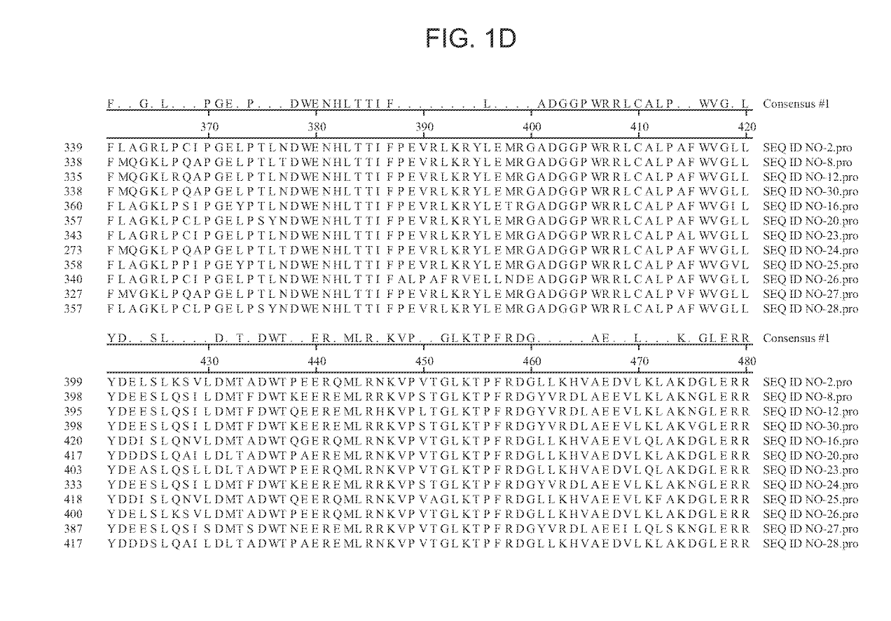

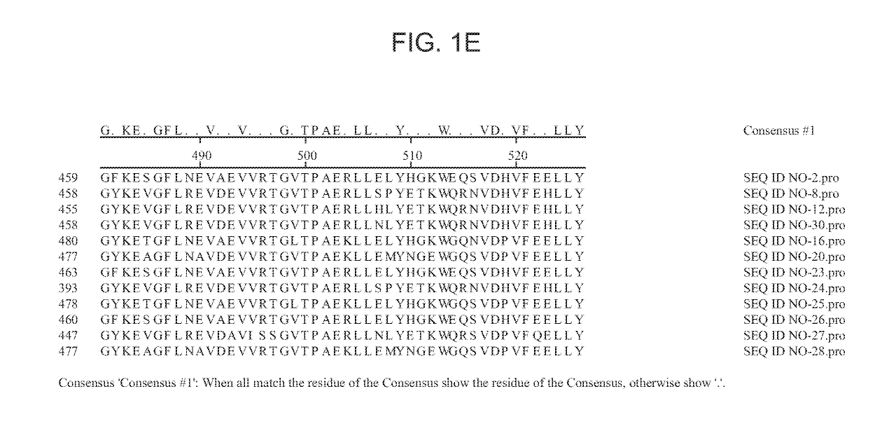

FIGS. 1A-1E present an alignment of the amino acid sequences of the GSH1 precursor polypeptides set forth in SEQ ID NOs:2, 8, 12, 30, 16, 20, 23, 25, 26, 27, 28 and the maize GSH1 polypeptide of SEQ ID NO:24 that lacks a transit peptide.

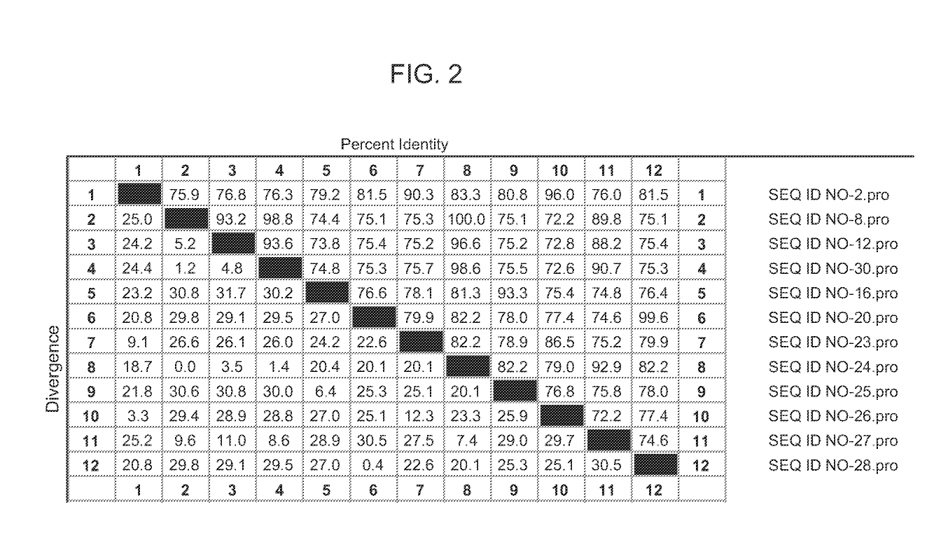

FIG. 2 shows the percent sequence identity and the divergence values for each pair of amino acids sequences displayed in FIGS. 1A-1E.

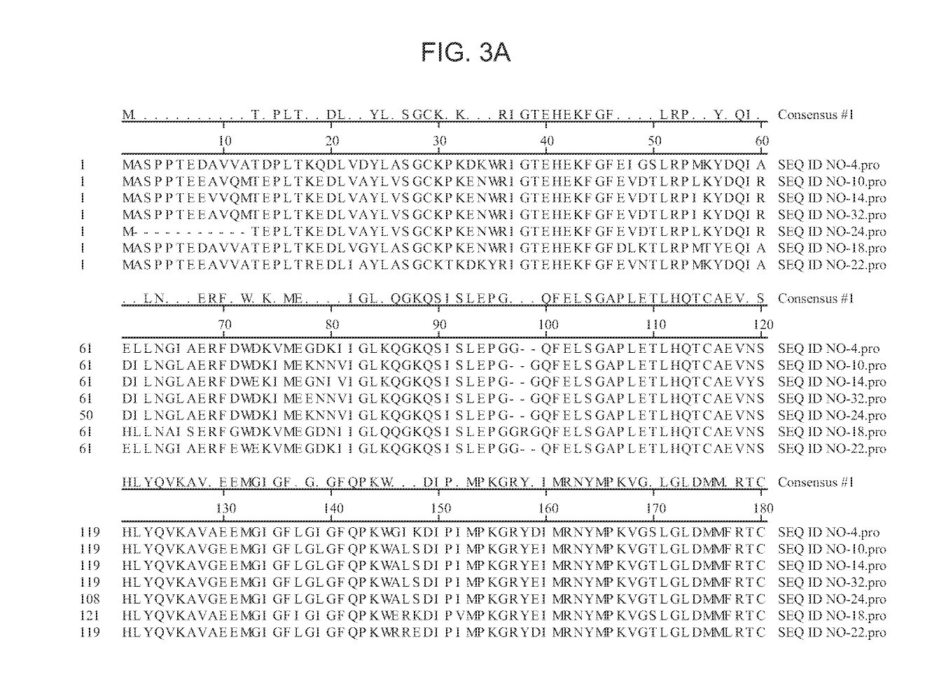

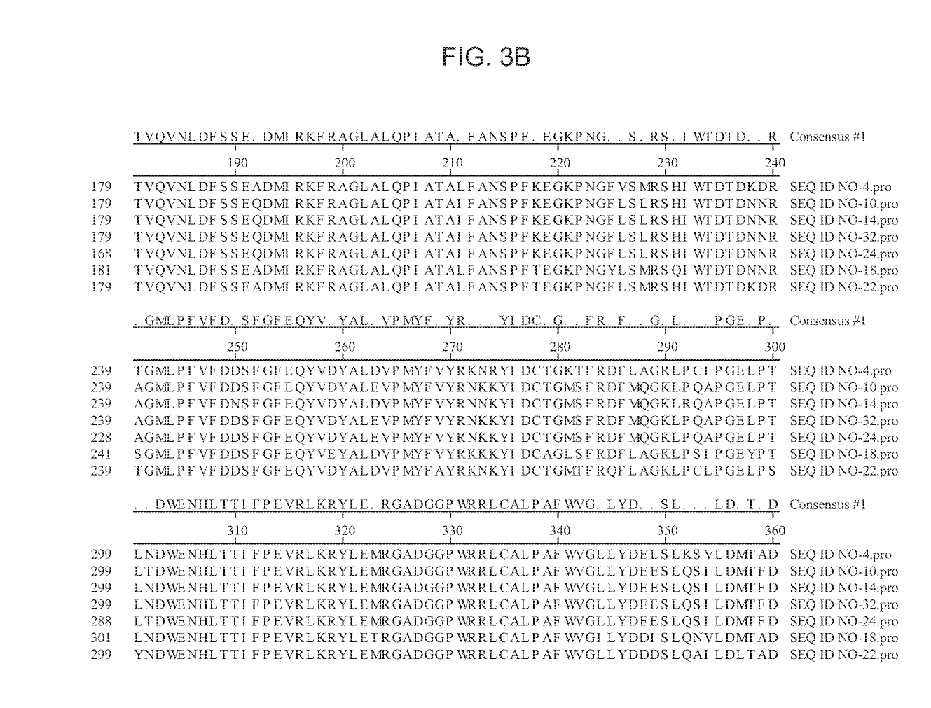

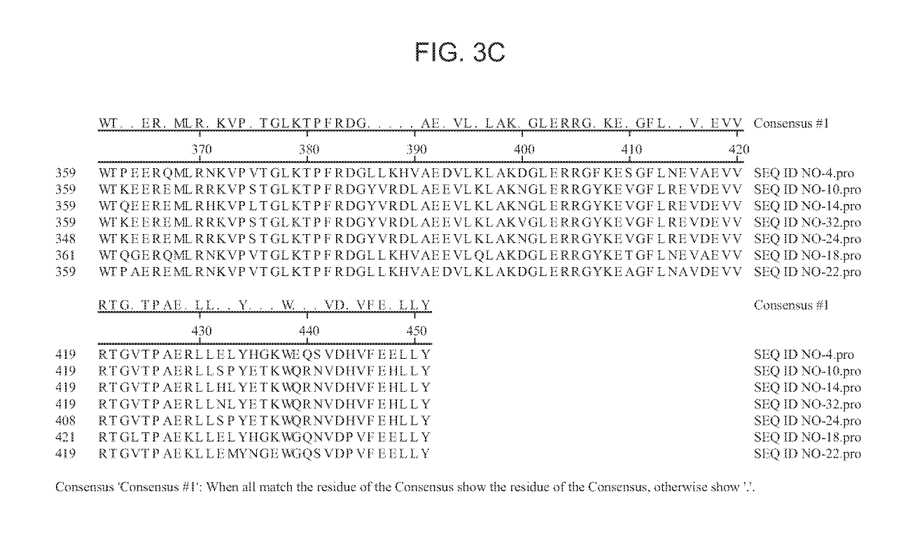

FIGS. 3A-3C present an alignment of the amino acid sequences of the GSH1 mature polypeptides set forth in SEQ ID NOs:4, 10, 14, 32, 18, 22 and the maize GSH1 polypeptide of SEQ ID NO:24 that lacks a transit peptide.

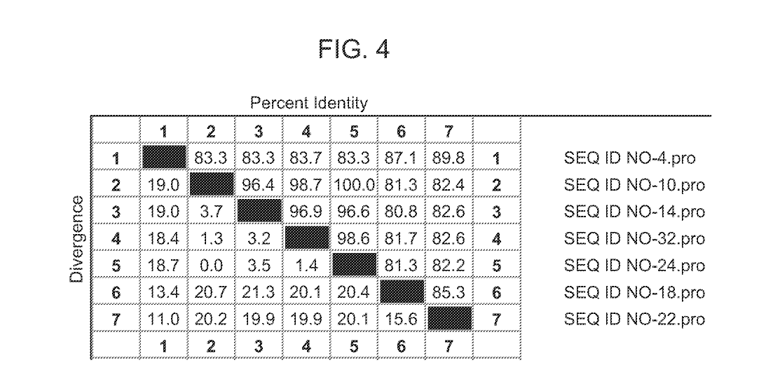

FIG. 4 shows the percent sequence identity and the divergence values for each pair of amino acids sequences displayed in FIGS. 3A-3C.

The multiple alignment of the sequences was performed using the MEGALIGN® program of the LASERGENE® bioinformatics computing suite (DNASTAR® Inc., Madison, Wis.); in particular, using the Clustal V method of alignment (Higgins and Sharp (1989) CABIOS. 5:151-153) with the multiple alignment default parameters of GAP PENALTY=10 and GAP LENGTH PENALTY=10, and the pairwise alignment default parameters of KTUPLE=1, GAP PENALTY=3, WINDOW=5 and DIAGONALS SAVED=5.

In another embodiment, a recombinant DNA construct comprises a polynucleotide operably linked to at least one regulatory sequence (e.g., a promoter functional in a plant), wherein said polynucleotide encodes a GSH1 polypeptide. For example, the GSH1 polypeptide may be from Arabidopsis thaliana, Zea mays, Glycine max, Glycine tabacina, Glycine soja and Glycine tomentella.

For a sequence encoding a chloroplast-localized precursor polypeptide, removal of the sequence encoding the transit peptide would be expected to result in production of a modified or “mature” polypeptide that is targeted to the cytoplasm. Embodiments of the current invention include both precursor GSH1 polypeptides that are targeted to the chloroplast and modified or mature GSH1 polypeptides that are targeted to the cytoplasm.

In another aspect, the present invention includes suppression DNA constructs.

A suppression DNA construct may comprise at least one regulatory sequence (for example, a promoter functional in a plant) operably linked to (a) all or part of: (i) a nucleic acid sequence encoding a polypeptide having an amino acid sequence of at least 50%, 51%, 52%, 53%, 54%, 55%, 56%, 57%, 58%, 59%, 60%, 56%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO:2, 4, 6, 8, 10, 12, 14, 16, 18, 20, 22, 23, 24, 25, 26, 27, 28, 30, 32, 43, 45, 47, 48, 49 and 50, or (ii) a full complement of the nucleic acid sequence of (a)(i); or (b) a region derived from all or part of a sense strand or antisense strand of a target gene of interest, said region having a nucleic acid sequence of at least 50%, 51%, 52%, 53%, 54%, 55%, 56%, 57%, 58%, 59%, 60%, 56%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to said all or part of a sense strand or antisense strand from which said region is derived, and wherein said target gene of interest encodes a GSH1 polypeptide; or (c) ail or part of: (i) a nucleic acid sequence of at least 50%, 51%, 52%, 53%, 54%, 55%, 56%, 57%, 58%, 59%, 60%, 56%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO:1, 3, 5, 7, 9, 11, 13, 15, 17, 19, 21, 29 or 31, or (ii) a full complement of the nucleic acid sequence of (c)(i). The suppression DNA construct may comprise a cosuppression construct, antisense construct, viral-suppression construct, hairpin suppression construct, stern-loop suppression construct, double-stranded RNA-producing construct, RNAi construct, or small RNA construct (e.g., an siRNA construct or an miRNA construct).

It is understood, as those skilled in the art will appreciate, that the invention encompasses more than the specific exemplary sequences. Alterations in a nucleic acid fragment which result in the production of a chemically equivalent amino acid at a given site, but do not affect the functional properties of the encoded polypeptide, are well known in the art. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the polypeptide molecule would also not be expected to alter the activity of the polypeptide. Each of the proposed modifications is well within the routine skill in the art, as is determination of retention of biological activity of the encoded products.

“Suppression DNA construct” is a recombinant DNA construct which when transformed or stably integrated into the genome of the plant, results in “silencing” of a target gene in the plant. The target gene may be endogenous or transgenic to the plant. “Silencing,” as used herein with respect to the target gene, refers generally to the suppression of levels of mRNA or protein/enzyme expressed by the target gene, and/or the level of the enzyme activity or protein functionality. The terms “suppression”, “suppressing” and “silencing”, used interchangeably herein, include lowering, reducing, declining, decreasing, inhibiting, eliminating or preventing. “Silencing” or “gene silencing” does not specify mechanism and is inclusive, and not limited to, anti-sense, cosuppression, viral-suppression, hairpin suppression, stem-loop suppression, RNAi-based approaches, and small RNA-based approaches.

A suppression DNA construct may comprise a region derived from a target gene of interest and may comprise all or part of the nucleic acid sequence of the sense strand (or antisense strand) of the target gene of interest. Depending upon the approach to be utilized, the region may be 100% identical or less than 100% identical (e.g., at least 50%, 51%, 52%, 53%, 54%, 55%, 56%, 57%, 58%, 59%, 60%, 56%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical) to all or part of the sense strand (or antisense strand) of the gene of interest.

Suppression DNA constructs are well-known in the art, are readily constructed once the target gene of interest is selected, and include, without limitation, cosuppression constructs, antisense constructs, viral-suppression constructs, hairpin suppression constructs, stem-loop suppression constructs, double-stranded RNA-producing constructs, and more generally, RNAi (RNA interference) constructs and small RNA constructs such as siRNA (short interfering RNA) constructs and miRNA (microRNA) constructs.

“Antisense inhibition” refers to the production of antisense RNA transcripts capable of suppressing the expression of the target gene or gene product. “Antisense RNA” refers to an RNA transcript that is complementary to all or part of a target primary transcript or mRNA and that blocks the expression of a target isolated nucleic acid fragment (U.S. Pat. No. 5,107,065). The complementarity of an antisense RNA may be with any part of the specific gene transcript, i.e., at the 5′ non-coding sequence, 3° non-coding sequence, introns, or the coding sequence.

“Cosuppression” refers to the production of sense RNA transcripts capable of suppressing the expression of the target gene or gene product. “Sense” RNA refers to RNA transcript that includes the mRNA and can be translated into protein within a cell or in vitro. Cosuppression constructs in plants have been previously designed by focusing on overexpression of a nucleic acid sequence having homology to a native mRNA, in the sense orientation, which results in the reduction of all RNA having homology to the overexpressed sequence (see Vaucheret et al., Plant J. 16:651-659 (1998); and Gura, Nature 404:804-808 (2000)).

Another variation describes the use of plant viral sequences to direct the suppression of proximal mRNA encoding sequences (POT Publication No. WO 98/36083 published on Aug. 20, 1998).

RNA interference refers to the process of sequence-specific post-transcriptional gene silencing in animals mediated by short interfering RNAs (siRNAs) (Fire et al. Nature 391:806 (1998)). The corresponding process in plants is commonly referred to as post-transcriptional gene silencing (PTGS) or RNA silencing and is also referred to as quelling in fungi. The process of post-transcriptional gene silencing is thought to be an evolutionarily-conserved cellular defense mechanism used to prevent the expression of foreign genes and is commonly shared by diverse flora and phyla (Fire at al., Trends Genet. 15:358 (1999)).

Small RNAs play an important role in controlling gene expression. Regulation of many developmental processes, including flowering, is controlled by small RNAs. It is now possible to engineer changes in gene expression of plant genes by using transgenic constructs which produce small RNAs in the plant.

Small RNAs appear to function by base-pairing to complementary RNA or DNA target sequences. When bound to RNA, small RNAs trigger either RNA cleavage or translational inhibition of the target sequence. When bound to DNA target sequences, it is thought that small RNAs can mediate DNA methylation of the target sequence. The consequence of these events, regardless of the specific mechanism, is that gene expression is inhibited.

MicroRNAs (miRNAs) are noncoding RNAs of about 19 to about 24 nucleotides (nt) in length that have been identified in both animals and plants (Lagos-Quintana et al., Science 294:853-858 (2001), Lagos-Quintana et al. Curr. Biol. 12:735-739 (2002); Lau et al., Science 294:858-862 (2001); Lee and Ambros, Science 294:862-864 (2001); Llave et al., Plant Cell 14:1605-1619 (2002); Mourelatos et al., Genes Dev, 16:720-728 (2002); Park et al., Curr. Biol. 12:1484-1495 (2002); Reinhart et al., Genes. Dev. 16:1616-1626 (2002)). They are processed from longer precursor transcripts that range in size from approximately 70 to 200 nt, and these precursor transcripts have the ability to form stable hairpin structures.

MicroRNAs (miRNAs) appear to regulate target genes by binding to complementary sequences located in the transcripts produced by these genes. It seems likely that miRNAs can enter at least two pathways of target gene regulation: (1) translational inhibition; and (2) RNA cleavage. MicroRNAs entering the RNA cleavage pathway are analogous to the 21-25 nt short interfering RNAs (siRNAs) generated during RNA interference (RNAi) in animals and posttranscriptional gene silencing (PTGS) in plants, and likely are incorporated into an RNA-induced silencing complex (RISC) that is similar or identical to that seen for RNAi.

Regulatory Sequences:

A recombinant DNA construct (including a suppression DNA construct) of the present invention may comprise at least one regulatory sequence.

A regulatory sequence may be a promoter.

A number of promoters can be used in recombinant DNA constructs of the present invention. The promoters can be selected based on the desired outcome, and may include constitutive, tissue-specific, inducible, or other promoters for expression in the host organism.

Promoters that cause a gene to be expressed in most cell types at most times are commonly referred to as “constitutive promoters”.

High level, constitutive expression of the candidate gene under control of the 355 or UBI promoter may have pleiotropic effects, although candidate gene efficacy may be estimated when driven by a constitutive promoter. Use of tissue-specific and/or stress-specific promoters may eliminate undesirable effects but retain the ability to enhance drought tolerance. This effect has been observed in Arabidopsis (Kasuga et al. (1999) Nature Biotechnol. 17:287-91).

Suitable constitutive promoters for use in a plant host cell include, for example, the core promoter of the Rsyn7 promoter and other constitutive promoters disclosed in WO 99/43838 and U.S. Pat. No. 6,072,050; the core CaMV 35S promoter (Odell et al., Nature 313:810-812 (1985)); rice actin (McElroy et al., Plant Cell 2:163-171 (1990)); ubiquitin (Christensen et al., Plant Mol, Biol. 12:619-632 (1989) and Christensen at al., Plant Mol. Biol. 18:675-689 (1992)); pEMU (Last et al., Theor. Appl. Genet. 81:581-588 (1991)); MAS (Velten et al., EMBO J. 3:2723-2730 (1984)); ALS promoter (U.S. Pat. No. 5,659,026), and the like. Other constitutive promoters include, for example, those discussed in U.S. Pat. Nos. 5,608,149; 5,608,144; 5,604,121; 5,569,597; 5,466,785; 5,399,680; 5,268,463; 5,608,142; and 6,177,611.

In choosing a promoter to use in the methods of the invention, it may be desirable to use a tissue-specific or developmentally regulated promoter.

For example, a tissue-specific or developmentally regulated promoter for use in the current invention may be a DNA sequence which regulates the expression of a DNA sequence selectively in the cells/tissues of a plant critical to tassel development, seed set, or both, and limits the expression of such a DNA sequence to the period of tassel development or seed maturation in the plant. Any identifiable promoter may be used in the methods of the present invention which causes the desired temporal and spatial expression.

Promoters which are seed or embryo-specific and may be useful in the invention include soybean Kunitz trypsin inhibitor (Kti3, Jofuku and Goldberg, Plant Cell 1:1079-1093 (1989)), patatin (potato tubers) (Rocha-Sosa, M., at al. (1989) EMBO J. 8:23-29), convicilin, vicilin, and legumin (pea cotyledons) (Rerie, W. G., et al. (1991) Mol. Gen. Genet. 259:149-157; Newbigin, E. J., et al. (1990) Planta 180:461-470; Higgins, T. J. V., et al. (1988) Plant. Mol. Biol. 11:683-695), zein (maize endosperm) (Schemthaner, J. P., et al. (1988) EMBO J. 7:1249-1255), phaseolin (bean cotyledon) (Segupta-Gopalan, C., et al. (1985) Proc. Natl. Acad. Sci. U.S.A. 82:3320-3324), phytohemagglutinin (bean cotyledon) (Voelker, T. at al. (1987) EMBO J. 6:3571-3577), B-conglycinin and glycinin (soybean cotyledon) (Chen, Z-L, et al. (1988) EMBO J. 7:297-302), glutelin (rice endosperm), hordein (barley endosperm) (Marris, C., et al. (1988) Plant Mol. Biol. 10:359-366), glutenin and gliadin (wheat endosperm) (Colot, V., et al. (1987) EMBO J. 6:3559-3564), and sporamin (sweet potato tuberous root) (Hattori, T., et al. (1990) Plant Mol. Biol. 14:595-604). Promoters of seed-specific genes operably linked to heterologous coding regions in chimeric gene constructions maintain their temporal and spatial expression pattern in transgenic plants. Such examples include Arabidopsis thaliana 2S seed storage protein gene promoter to express enkephalin peptides in Arabidopsis and Brassica napus seeds (Vanderkerckhove et al., Bio/Technology 7:L929-932 (1989)), bean lectin and bean beta-phaseolin promoters to express luciferase (Riggs at al., Plant Sci. 63:47-57 (1989)), and wheat glutenin promoters to express chloramphenicol acetyl transferase (Colot et al., EMBO J 6:3559-3564 (1987)).

Inducible promoters selectively express an operably linked DNA sequence in response to the presence of an endogenous or exogenous stimulus, for example by chemical compounds (chemical inducers) or in response to environmental, hormonal, chemical, and/or developmental signals. Inducible or regulated promoters include, for example, promoters regulated by light, heat, stress, flooding or drought, phytohormones, wounding, or chemicals such as ethanol, jasmonate, salicylic acid, or safeners.

Promoters for use in the current invention include the following: 1) the stress-inducible RD29A promoter (Kasuga at al. (1999) Nature Biotechnol. 17:28 T-91); 2) the barley promoter, B22E; expression of B22E is specific to the pedicel in developing maize kernels (“Primary Structure of a Novel Barley Gene Differentially Expressed in Immature Aleurone Layers”. Klemsdal, S. S. et al., Mol. Gen. Genet. 228(1/2):9-16 (1991)); and 3) maize promoter, Zag2 (“Identification and molecular characterization of ZAG1, the maize homolog of the Arabidopsis floral homeotic gene AGAMOUS”, Schmidt, R. J. et al., Plant Cell 5(7):729-737 (1993); “Structural characterization, chromosomal localization and phylogenetic evaluation of two pairs of AGAMOUS-like MADS-box genes from maize”, Theissen et al. Gene 156(2):155-166 (1995); NCBI GenBank Accession No. X80206)). Zag2 transcripts can be detected 5 days prior to pollination to 7 to 8 days after pollination (“DAP”), and directs expression in the carpel of developing female inflorescences and Ciml which is specific to the nucleus of developing maize kernels. Ciml transcript is detected 4 to 5 days before pollination to 6 to 8 DAP. Other useful promoters include any promoter which can be derived from a gene whose expression is maternally associated with developing female florets.

Additional promoters for regulating the expression of the nucleotide sequences of the present invention in plants are stalk-specific promoters. Such stalk-specific promoters include the alfalfa S2A promoter (GenBank Accession No. EF030816; Abrahams et al., Plant Mol. Biol. 27:513-528 (1995)) and S2B promoter (GenBank Accession No. EF030817) and the like, herein incorporated by reference.

Promoters may be derived in their entirety from a native gene, or be composed of different elements derived from different promoters found in nature, or even comprise synthetic DNA segments.

Promoters for use in the current invention may include: RIP2, mLIP15, ZmCOR1, Rab17, CaMV 35S, RD29A, B22E, Zag2, SAM synthetase, ubiquitin, CaMV 19S, nos, Adh, sucrose synthase. R-allele, the vascular tissue preferred promoters S2A (Genbank accession number EF030816) and S2B (Genbank accession number EF030817), and the constitutive promoter GOS2 from Zea mays. Other promoters include root preferred promoters, such as the maize NAS2 promoter, the maize Cyclo promoter (US 200610156439, published Jul. 13, 2006), the maize ROOTMET2 promoter (WO05063998, published Jul. 14, 2005), the CR1BIO promoter (WO06055487, published May 26, 2006), the CRWAQ81 (WO05035770, published Apr. 21, 2005) and the maize ZRP2.47 promoter (NCBI accession number: U38790; GI No. 1063664),

Recombinant DNA constructs of the present invention may also include other regulatory sequences, including but not limited to, translation leader sequences, introns, and polyadenylation recognition sequences. In another embodiment of the present invention, a recombinant DNA construct of the present invention further comprises an enhancer or silencer.

An intron sequence can be added to the 5 untranslated region, the protein-coding region or the 3′ untranslated region to increase the amount of the mature message that accumulates in the cytosol. Inclusion of a spliceable intron in the transcription unit in both plant and animal expression constructs has been shown to increase gene expression at both the mRNA and protein levels up to 1000-fold. Buchman and Berg, Mol. Cell Biol. 8:4395-4405 (1988); Callis et al., Genes Dev. 1:1183-1200 (1987).

Any plant can be selected for the identification of regulatory sequences and GSH1 polypeptide genes to be used in recombinant DNA constructs of the present invention. Examples of suitable plant targets for the isolation of genes and regulatory sequences would include but are not limited to alfalfa, apple, apricot, Arabidopsis, artichoke, arugula, asparagus, avocado, banana, barley, beans, beet, blackberry, blueberry, broccoli, brussels sprouts, cabbage, canola, cantaloupe, carrot, cassava, castorbean, cauliflower, celery, cherry, chicory, cilantro, citrus, clementines, clover, coconut, coffee, corn, cotton, cranberry, cucumber, Douglas fir, eggplant, endive, escarole, eucalyptus, fennel, figs, garlic, gourd, grape, grapefruit, honey dew, jicama, kiwifruit, lettuce, leeks, lemon, lime, Loblolly pine, linseed, mango, melon, mushroom, nectarine, nut, oat, oil palm, oil seed rape, okra, olive, onion, orange, an ornamental plant, palm, papaya, parsley, parsnip, pea, peach, peanut, pear, pepper, persimmon, pine, pineapple, plantain, plum, pomegranate, poplar, potato, pumpkin, quince, radiate pine, radicchio, radish, rapeseed, raspberry, rice, rye, sorghum, Southern pine, soybean, spinach, squash, strawberry, sugarbeet, sugarcane, sunflower, sweet potato, sweetgum, switchgrass, tangerine, tea, tobacco, tomato, triticale, turf, turnip, a vine, watermelon, wheat, yams, and zucchini.

Compositions:

A composition of the present invention is a plant comprising in its genome any of the recombinant DNA constructs (including any of the suppression DNA constructs) of the present invention (such as any of the constructs discussed above). Compositions also include any progeny of the plant, and any seed obtained from the plant or its progeny, wherein the progeny or seed comprises within its genome the recombinant DNA construct (or suppression DNA construct). Progeny includes subsequent generations obtained by self-pollination or out-crossing of a plant. Progeny also includes hybrids and inbreds.

In hybrid seed propagated crops, mature transgenic plants can be self-pollinated to produce a homozygous inbred plant. The inbred plant produces seed containing the newly introduced recombinant DNA construct (or suppression DNA construct). These seeds can be grown to produce plants that would exhibit an altered agronomic characteristic (e.g., an increased agronomic characteristic optionally under water limiting or nitrogen limiting conditions), or used in a breeding program to produce hybrid seed, which can be grown to produce plants that would exhibit such an altered agronomic characteristic. The seeds may be maize or rice seeds.

The plant is a monocotyledonous or dicotyledonous plant, for example, a maize, rice or soybean plant. The plant may be a maize hybrid plant, a rice hybrid plant, a maize inbred plant or a rice inbred plant. The plant may also be sunflower, sorghum, canola, wheat, alfalfa, cotton, barley, millet, sugar cane or switchgrass.

The recombinant DNA construct may be stably integrated into the genome of the plant.

Particularly embodiments include but are not limited to the following embodiments:

1. A plant (for example, a maize, rice or soybean plant) comprising in its genome a recombinant DNA construct comprising a polynucleotide operably linked to at least one regulatory sequence, wherein said polynucleotide encodes a polypeptide having an amino acid sequence of at least 50%, 51%, 52%, 53%, 54%, 55%, 56%, 57%, 58%, 59%, 60%, 56%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO:2, 4, 6, 8, 10, 12, 14, 16, 18, 20, 22, 23, 24, 25, 26, 27, 28, 30, 32, 43, 45, 47, 48, 49 and 50, and wherein said plant exhibits increased drought tolerance when compared to a control plant not comprising said recombinant DNA construct. The plant further may exhibit an alteration of at least one agronomic characteristic when compared to the control plant.

2. A plant (for example, a maize, rice or soybean plant) comprising in its genome a recombinant DNA construct comprising a polynucleotide operably linked to at least one regulatory sequence, wherein said polynucleotide encodes a GSH1 polypeptide, and wherein said plant exhibits increased drought tolerance when compared to a control plant not comprising said recombinant DNA construct. The plant further may exhibit an alteration of at least one agronomic characteristic when compared to the control plant.

3. A plant (for example, a maize, rice or soybean plant) comprising in its genome a recombinant DNA construct comprising a polynucleotide operably linked to at least one regulatory sequence, wherein said polynucleotide encodes a GSH1 polypeptide, and wherein said plant exhibits an alteration of at least one agronomic characteristic when compared to a control plant not comprising said recombinant DNA construct.

4. A plant (for example, a maize, rice or soybean plant) comprising in its genome a recombinant DNA construct comprising a polynucleotide operably linked to at least one regulatory element, wherein said polynucleotide encodes a polypeptide having an amino add sequence of at least 50%, 51%, 52%, 53%, 54%, 55%, 56%, 57%, 58%, 59%, 60%, 56%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO:2, 4, 6, 8, 10, 12, 14, 16, 18, 20, 22, 23, 24, 25, 26, 27, 28, 30, 32, 43, 45, 47, 48, 49 and 50, and wherein said plant exhibits an alteration of at least one agronomic characteristic when compared to a control plant not comprising said recombinant DNA construct.

5. A plant (for example, a maize, rice or soybean plant) comprising in its genome a suppression DNA construct comprising at least one regulatory element operably linked to a region derived from all or part of a sense strand or antisense strand of a target gene of interest, said region having a nucleic acid sequence of at least 50%, 51%, 52%, 53%, 54%, 55%, 56%, 57%, 58%, 59%, 60%, 56%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to said all or part of a sense strand or antisense strand from which said region is derived, and wherein said target gene of interest encodes a GSH1 polypeptide, and wherein said plant exhibits an alteration of at least one agronomic characteristic when compared to a control plant not comprising said suppression DNA construct.

6. A plant (for example, a maize, rice or soybean plant) comprising in its genome a suppression DNA construct comprising at least one regulatory element operably linked to all or part of (a) a nucleic acid sequence encoding a polypeptide having an amino acid sequence of at least 50%, 51%, 52%, 53%, 54%, 55%, 56%, 57%, 58%, 59%, 60%, 56%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO:2, 4, 6, 8, 10, 12, 14, 16, 18, 20, 22, 23, 24, 25, 26, 27, 28, 30, 32, 43, 45, 47, 48, 49 and 50, or (b) a full complement of the nucleic acid sequence of (a), and wherein said plant exhibits an alteration of at least one agronomic characteristic when compared to a control plant not comprising said suppression DNA construct.

7. Any progeny of the above plants in embodiments 1-6, any seeds of the above plants in embodiments 1-6, any seeds of progeny of the above plants in embodiments 1-6, and cells from any of the above plants in embodiments 1-6 and progeny thereof.

In any of the foregoing embodiments 1-7 or any other embodiments of the present invention, the GSH1 polypeptide may be from Arabidopsis thaliana, Zea mays, Oryza sativa, Glycine max, Glycine tabacina, Glycine soja or Glycine tomentella.

In any of the foregoing embodiments 1-7 or any other embodiments of the present invention, the recombinant DNA construct (or suppression DNA construct) may comprise at least a promoter functional in a plant as a regulatory sequence.

In any of the foregoing embodiments 1-7 or any other embodiments of the present invention, the alteration of at least one agronomic characteristic is either an increase or decrease.

In any of the foregoing embodiments 1-7 or any other embodiments of the present invention, the at least one agronomic characteristic may be selected from the group consisting of greenness, yield, growth rate, biomass, fresh weight at maturation, dry weight at maturation, fruit yield, seed yield, total plant nitrogen content, fruit nitrogen content, seed nitrogen content, nitrogen content in a vegetative tissue, total plant free amino acid content, fruit free amino acid content, seed free amino acid content, free amino acid content in a vegetative tissue, total plant protein content, fruit protein content, seed protein content, protein content in a vegetative tissue, drought tolerance, nitrogen uptake, root lodging, harvest index, stalk lodging, plant height, ear height, ear length, salt tolerance, early seedling vigor and seedling emergence under low temperature stress. For example, the alteration of at least one agronomic characteristic may be an increase in yield, greenness or biomass.

In any of the foregoing embodiments 1-7 or any other embodiments of the present invention, the plant may exhibit the alteration of at least one agronomic characteristic when compared, under water limiting conditions or nitrogen limiting conditions, or both, to a control plant not comprising said recombinant DNA construct (or said suppression DNA construct).

In any of the foregoing embodiments 1-7 or any other embodiments of the present invention, the plant may exhibit an alteration in root architecture when compared to said control plant.

“Nitrogen limiting conditions” refers to conditions where the amount of total available nitrogen (e.g., from nitrates, ammonia, or other known sources of nitrogen) is not sufficient to sustain optimal plant growth and development. One skilled in the art would recognize conditions where total available nitrogen is sufficient to sustain optimal plant growth and development. One skilled in the art would recognize what constitutes sufficient amounts of total available nitrogen, and what constitutes soils, media and fertilizer inputs for providing nitrogen to plants. Nitrogen limiting conditions will vary depending upon a number of factors, including but not limited to, the particular plant and environmental conditions. “Nitrogen stress tolerance” is a trait of a plant and refers to the ability of the plant to survive under nitrogen limiting conditions.

“Increased nitrogen stress tolerance” of a plant is measured relative to a reference or control plant, and means that the nitrogen stress tolerance of the plant is increased by any amount or measure when compared to the nitrogen stress tolerance of the reference or control plant.

A “nitrogen stress tolerant plant” is a plant that exhibits nitrogen stress tolerance. A nitrogen stress tolerant plant may be a plant that exhibits an increase in at least one agronomic characteristic relative to a control plant under nitrogen limiting conditions.

The term “root architecture” refers to the arrangement of the different parts that comprise the root. The terms “root architecture”, “root structure”, “root system” or “root system architecture” are used interchangeably herein.

In general, the first root of a plant that develops from the embryo is called the primary root. In most dicots, the primary root is called the taproot. This main root grows downward and gives rise to branch (lateral) roots. In monocots the primary root of the plant branches, giving rise to a fibrous root system.

The term “altered root architecture” refers to aspects of alterations of the different parts that make up the root system at different stages of its development compared to a reference or control plant. It is understood that altered root architecture encompasses alterations in one or more measurable parameters, including but not limited to, the diameter, length, number, angle or surface of one or more of the root system parts, including but not limited to, the primary root, lateral or branch root, adventitious root, and root hafts, all of which fall within the scope of this invention. These changes can lead to an overall alteration in the area or volume occupied by the root. The reference or control plant does not comprise in its genome the recombinant DNA construct or heterologous construct.

“Environmental conditions” refer to conditions under which the plant is grown, such as the availability of water, availability of nutrients (for example nitrogen), or the presence of insects or disease.

“Drought” refers to a decrease in water availability to a plant that, especially when prolonged, can cause damage to the plant or prevent its successful growth (e.g., limiting plant growth or seed yield).

“Drought tolerance” is a trait of a plant to survive under drought conditions over prolonged periods of time without exhibiting substantial physiological or physical deterioration.

“Drought tolerance activity” of a polypeptide indicates that over-expression of the polypeptide in a transgenic plant confers increased drought tolerance to the transgenic plant relative to a reference or control plant.

“Increased drought tolerance” of a plant is measured relative to a reference or control plant, and is a trait of the plant to survive under drought conditions over prolonged periods of time, without exhibiting the same degree of physiological or physical deterioration relative to the reference or control plant grown under similar drought conditions. Typically, when a transgenic plant comprising a recombinant DNA construct or suppression DNA construct in its genome exhibits increased drought tolerance relative to a reference or control plant, the reference or control plant does not comprise in its genome the recombinant DNA construct or suppression DNA construct.

One of ordinary skill in the an is familiar with protocols for simulating drought conditions and for evaluating drought tolerance of plants that have been subjected to simulated or naturally-occurring drought conditions. For example, one can simulate drought conditions by giving plants less water than normally required or no water over a period of time, and one can evaluate drought tolerance by looking for differences in physiological and/or physical condition, including (but not limited to) vigor, growth, size, or root length, or in particular, leaf color or leaf area size. Other techniques for evaluating drought tolerance include measuring chlorophyll fluorescence, photosynthetic rates and gas exchange rates.

A drought stress experiment may involve a chronic stress (i.e., slow dry down) and/or may involve two acute stresses (i.e., abrupt removal of water) separated by a day or two of recovery. Chronic stress may last 8-10 days. Acute stress may last 3-5 days. The following variables may be measured during drought stress and well watered treatments of transgenic plants and relevant control plants:

The variable “% area chg_start chronic—acute2” is a measure of the percent change in total area determined by remote visible spectrum imaging between the first day of chronic stress and the day of the second acute stress.

The variable “% area chg_start chronic—end chronic” is a measure of the percent change in total area determined by remote visible spectrum imaging between the first day of chronic stress and the last day of chronic stress.

The variable “% area chg_start chronic—harvest” is a measure of the percent change in total area determined by remote visible spectrum imaging between the first day of chronic stress and the day of harvest.

The variable “% area chg_start chronic—recovery24 hr” is a measure of the percent change in total area determined by remote visible spectrum imaging between the first day of chronic stress and 24 his into the recovery (24 hrs after acute stress 2).

The variable “psii_acute1” is a measure of Photosystem II (PSII) efficiency at the end of the first acute stress period. It provides an estimate of the efficiency at which light is absorbed by PSII antennae and is directly related to carbon dioxide assimilation within the leaf.

The variable “psii_acute2” is a measure of Photosystem II (PSII) efficiency at the end of the second acute stress period. It provides an estimate of the efficiency at which light is absorbed by PSII antennae and is directly related to carbon dioxide assimilation within the leaf.

The variable “fv/fm_acute1” is a measure of the optimum quantum yield (Fv/Fm) at the end of the first acute stress—(variable fluorescence difference between the maximum and minimum fluorescence/maximum fluorescence).

The variable “fv/fm_acute2” is a measure of the optimum quantum yield (Fv/Fm) at the end of the second acute stress—(variable flourescence difference between the maximum and minimum fluorescence maximum fluorescence).

The variable “leaf rolling_harvest” is a measure of the ratio of top image to side image on the day of harvest.

The variable “leaf rolling_recovery24 hr” is a measure of the ratio of top image to side image 24 hours into the recovery.

The variable “Specific Growth Rate (SGR)” represents the change in total plant surface area (as measured by Lemna Tec Instrument) over a single day (Y(t)=Y0*er*t), Y(t)=Y0*er*t is equivalent to % change in Y/Δ t where the individual terms are as follows: Y(t)=Total surface area at t; Y0=Initial total surface area (estimated); r=Specific Growth Rate day-1, and t=Days After Planting (“DAP”).

The variable “shoot dry weight” is a measure of the shoot weight 96 hours after being placed into a 104° C. oven.

The variable “shoot fresh weight” is a measure of the shoot weight immediately after being cut from the plant.

The Examples below describe some representative protocols and techniques for simulating drought conditions and/or evaluating drought tolerance.

One can also evaluate drought tolerance by the ability of a plant to maintain sufficient yield (for example, at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% yield) in field testing under simulated or naturally-occurring drought conditions (e.g., by measuring for substantially equivalent yield under drought conditions compared to non-drought conditions, or by measuring for less yield loss under drought conditions compared to a control or reference plant).

One of ordinary skill in the art would readily recognize a suitable control or reference plant to be utilized when assessing or measuring an agronomic characteristic or phenotype of a transgenic plant in any embodiment of the present invention in which a control or reference plant is utilized (e.g., compositions or methods as described herein). For example, by way of non-limiting illustrations:

1. Progeny of a transformed plant which is hemizygous with respect to a recombinant DNA construct (or suppression DNA construct), such that the progeny are segregating into plants either comprising or not comprising the recombinant DNA construct (or suppression DNA construct): the progeny comprising the recombinant DNA construct (or suppression DNA construct) would be typically measured relative to the progeny not comprising the recombinant DNA construct (or suppression DNA construct) (i.e., the progeny not comprising the recombinant DNA construct (or the suppression DNA construct) is the control or reference plant).

2. Introgression of a recombinant DNA construct (or suppression DNA construct) into an inbred line, such as in maize, or into a variety, such as in soybean: the introgressed line would typically be measured relative to the parent inbred or variety line (i.e., the parent inbred or variety line is the control or reference plant).

3. Two hybrid lines, where the first hybrid line is produced from two parent inbred lines, and the second hybrid line is produced from the same two parent inbred lines except that one of the parent inbred lines contains a recombinant DNA construct (or suppression DNA construct): the second hybrid line would typically be measured relative to the first hybrid line (i.e., the first hybrid line is the control or reference plant).

4. A plant comprising a recombinant DNA construct (or suppression DNA construct): the plant may be assessed or measured relative to a control plant not comprising the recombinant DNA construct (or suppression DNA construct) but otherwise having a comparable genetic background to the plant (e.g., sharing at least 90%, 91%, 92%, 93%, 94%, 95%, 96%, 97%, 98%, 99%, or 100% sequence identity of nuclear genetic material compared to the plant comprising the recombinant DNA construct (or suppression DNA construct)). There are many laboratory-based techniques available for the analysis, comparison and characterization of plant genetic backgrounds; among these are Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length Polymorphisms (AFLP®s), and Simple Sequence Repeats (SSRs) which are also referred to as Microsatellites.

Furthermore, one of ordinary skill in the art would readily recognize that a suitable control or reference plant to be utilized when assessing or measuring an agronomic characteristic or phenotype of a transgenic plant would not include a plant that had been previously selected, via mutagenesis or transformation, for the desired agronomic characteristic or phenotype.

Methods:

Methods include but are not limited to methods for increasing drought tolerance in a plant, methods for evaluating drought tolerance in a plant, methods for altering an agronomic characteristic in a plant, methods for determining an alteration of an agronomic characteristic in a plant, and methods for producing seed. The plant is a monocotyledonous or dicotyledonous plant, for example, a maize, rice or soybean plant. The plant may also be sunflower, sorghum, canola, wheat, alfalfa, cotton, barley, millet, sugar cane or sorghum. The seed may be a maize, rice or soybean seed, such as, a maize or rice hybrid seed or a maize or rice inbred seed.

Methods include but are not limited to the following:

A method for transforming a cell comprising transforming a cell with any of the isolated polynucleotides of the present invention. The cell transformed by this method is also included. In particular embodiments, the cell is eukaryotic cell, e.g., a yeast, insect or plant cell, or prokaryotic, e.g., a bacterial cell.

A method for producing a transgenic plant comprising transforming a plant cell with any of the isolated polynucleotides or recombinant DNA constructs (including suppression DNA constructs) of the present invention and regenerating a transgenic plant from the transformed plant cell. The invention is also directed to the transgenic plant produced by this method, and transgenic seed obtained from this transgenic plant. The transgenic plant obtained by this method may be used in other methods of the present invention.

A method for isolating a polypeptide of the invention from a cell or culture medium of the cell, wherein the cell comprises a recombinant DNA construct comprising a polynucleotide of the invention operably linked to at least one regulatory sequence, and wherein the transformed host cell is grown under conditions that are suitable for expression of the recombinant DNA construct.

A method of altering the level of expression of a polypeptide of the invention in a host cell comprising: (a) transforming a host cell with a recombinant DNA construct of the present invention; and (b) growing the transformed host cell under conditions that are suitable for expression of the recombinant DNA construct wherein expression of the recombinant DNA construct results in production of altered levels of the polypeptide of the invention in the transformed host cell.

A method of increasing drought tolerance in a plant, comprising: (a) introducing into a regenerable plant cell a recombinant DNA construct comprising a polynucleotide operably linked to at least one regulatory sequence (for example, a promoter functional in a plant), wherein the polynucleotide encodes a polypeptide having an amino acid sequence of at least 50%, 51%, 52%, 53%, 54%, 55%, 56%, 57%, 58%, 59%, 60%, 56%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO:2, 4, 6, 8, 10, 12, 14, 16, 18, 20, 22, 23, 24, 25, 26, 27, 28, 30, 32, 43, 45, 47, 48, 49 and 50; and (b) regenerating a transgenic plant from the regenerable plant cell after step (a), wherein the transgenic plant comprises in its genome the recombinant DNA construct and exhibits increased drought tolerance when compared to a control plant not comprising the recombinant DNA construct. The method may further comprise (c) obtaining a progeny plant derived from the transgenic plant, wherein said progeny plant comprises in its genome the recombinant DNA construct and exhibits increased drought tolerance when compared to a control plant not comprising the recombinant DNA construct.

A method of evaluating drought tolerance in a plant, comprising (a) obtaining a transgenic plant, wherein the transgenic plant comprises in its genome a recombinant DNA construct comprising a polynucleotide operably linked to at least one regulatory sequence (for example, a promoter functional in a plant), wherein said polynucleotide encodes a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO:2, 4, 6, 8, 10, 12, 14, 16, 18, 20, 22, 23, 24, 25, 26, 27, 28, 30, 32, 43, 45, 47, 48, 49 and 50; (b) obtaining a progeny plant derived from said transgenic plant, wherein the progeny plant comprises in its genome the recombinant DNA construct; and (c) evaluating the progeny plant for drought tolerance compared to a control plant not comprising the recombinant DNA construct.

A method of evaluating drought tolerance in a plant, comprising (a) obtaining a transgenic plant, wherein the transgenic plant comprises in its genome a suppression DNA construct comprising at least one regulatory sequence (for example, a promoter functional in a plant) operably linked to all or part of (i) a nucleic acid sequence encoding a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO:2, 4, 6, 8, 10, 12, 14, 16, 18, 20, 22, 23, 24, 25, 26, 27, 28, 30, 32, 43, 45, 47, 48, 49 and 50, or (ii) a full complement of the nucleic acid sequence of (a)(i); (b) obtaining a progeny plant derived from said transgenic plant, wherein the progeny plant comprises in its genome the suppression DNA construct; and (c) evaluating the progeny plant for drought tolerance compared to a control plant not comprising the suppression DNA construct.

A method of evaluating drought tolerance in a plant, comprising (a) obtaining a transgenic plant, wherein the transgenic plant comprises in its genome a suppression DNA construct comprising at least one regulatory sequence (for example, a promoter functional in a plant) operably linked to a region derived from all or part of a sense strand or antisense strand of a target gene of interest, said region having a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to said all or part of a sense strand or antisense strand from which said region is derived, and wherein said target gene of interest encodes a GSH1 polypeptide; (b) obtaining a progeny plant derived from the transgenic plant, wherein the progeny plant comprises in its genome the suppression DNA construct; and (c) evaluating the progeny plant for drought tolerance compared to a control plant not comprising the suppression DNA construct.

A method of determining an alteration of an agronomic characteristic in a plant, comprising (a) obtaining a transgenic plant, wherein the transgenic plant comprises in its genome a recombinant DNA construct comprising a polynucleotide operably linked to at least one regulatory sequence (for example, a promoter functional in a plant), wherein said polynucleotide encodes a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO:2, 4, 6, 8, 10, 12, 14, 16, 18, 20, 22, 23, 24, 25, 26, 27, 28, 30, 32, 43, 45, 47, 48, 49 and 50; (b) obtaining a progeny plant derived from said transgenic plant, wherein the progeny plant comprises in its genome the recombinant DNA construct; and (c) determining whether the progeny plant exhibits an alteration in at least one agronomic characteristic when compared, optionally under water limiting conditions, to a control plant not comprising the recombinant DNA construct.

A method of determining an alteration of an agronomic characteristic in a plant, comprising (a) obtaining a transgenic plant, wherein the transgenic plant comprises in its genome a suppression DNA construct comprising at least one regulatory sequence (for example, a promoter functional in a plant) operably linked to all or part of (i) a nucleic acid sequence encoding a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO:2, 4, 6, 8, 10, 12, 14, 16, 18, 20, 22, 23, 24, 25, 26, 27, 28, 30, 32, 43, 45, 47, 48, 49 and 50, or (ii) a full complement of the nucleic acid sequence of (i); (b) obtaining a progeny plant derived from said transgenic plant, wherein the progeny plant comprises in its genome the suppression DNA construct; and (c) determining whether the progeny plant exhibits an alteration in at least one agronomic characteristic when compared, optionally under water limiting conditions, to a control plant not comprising the suppression DNA construct.

A method of determining an alteration of an agronomic characteristic in a plant, comprising (a) obtaining a transgenic plant, wherein the transgenic plant comprises in its genome a suppression DNA construct comprising at least one regulatory sequence (for example, a promoter functional in a plant) operably linked to a region derived from all or part of a sense strand or antisense strand of a target gene of interest, said region having a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to said all or part of a sense strand or antisense strand from which said region is derived, and wherein said target gene of interest encodes a GSH1 polypeptide; (b) obtaining a progeny plant derived from said transgenic plant, wherein the progeny plant comprises in its genome the suppression DNA construct; and (c) determining whether the progeny plant exhibits an alteration in at least one agronomic characteristic when compared, optionally under water limiting conditions, to a control plant not comprising the suppression DNA construct.

A method of producing seed (for example, seed that can be sold as a drought tolerant product offering) comprising any of the preceding methods, and further comprising obtaining seeds from said progeny plant, wherein said seeds comprise in their genome said recombinant DNA construct (or suppression DNA construct).

In any of the preceding methods or any other embodiments of methods of the present invention, in said introducing step said regenerable plant cell may comprise a callus cell, an embryogenic callus cell, a gametic cell, a meristematic cell, or a cell of an immature embryo. The regenerable plant cells may be from an inbred maize plant or an inbred rice plant.

In any of the preceding methods or any other embodiments of methods of the present invention, said regenerating step may comprise the following: (i) culturing said transformed plant cells in a media comprising an embryogenic promoting hormone until callus organization is observed; (ii) transferring said transformed plant cells of step (i) to a first media which includes a tissue organization promoting hormone; and (iii) subculturing said transformed plant cells after step (ii) onto a second media, to allow for shoot elongation, root development or both.

In any of the preceding methods or any other embodiments of methods of the present invention, the at least one agronomic characteristic may be selected from the group consisting of greenness, yield, growth rate, biomass, fresh weight at maturation, dry weight at maturation, fruit yield, seed yield, total plant nitrogen content, fruit nitrogen content, seed nitrogen content, nitrogen content in a vegetative tissue, total plant free amino acid content, fruit free amino acid content, seed free amino acid content, amino acid content in a vegetative tissue, total plant protein content, fruit protein content, seed protein content, protein content in a vegetative tissue, drought tolerance, nitrogen uptake, root lodging, harvest index, stalk lodging, plant height, ear height, ear length, salt tolerance, early seedling vigor and seedling emergence under low temperature stress. The alteration of at least one agronomic characteristic may be an increase in yield, greenness or biomass.

In any of the preceding methods or any other embodiments of methods of the present invention, the plant may exhibit the alteration of at least one agronomic characteristic when compared, under water limiting conditions or nitrogen limiting conditions, to a control plant not comprising said recombinant DNA construct (or said suppression DNA construct).

In any of the preceding methods or any other embodiments of methods of the present invention, alternatives exist for introducing into a regenerable plant cell a recombinant DNA construct comprising a polynucleotide operably linked to at least one regulatory sequence. For example, one may introduce into a regenerable plant cell a regulatory sequence (such as one or more enhancers, for example, as part of a transposable element), and then screen for an event in which the regulatory sequence is operably linked to an endogenous gene encoding a polypeptide of the instant invention.

The introduction of recombinant DNA constructs of the present invention into plants may be carried out by any suitable technique, including but not limited to direct DNA uptake, chemical treatment, electroporation, microinjection, cell fusion, infection, vector-mediated DNA transfer, bombardment, or Agrobacterium-mediated transformation. Techniques for plant transformation and regeneration have been described in International Patent Publication WO 20091006276, the contents of which are herein incorporated by reference.

The development or regeneration of plants containing the foreign, exogenous isolated nucleic acid fragment that encodes a protein of interest is well known in the art. The regenerated plants may be self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants. A transgenic plant of the present invention containing a desired polypeptide is cultivated using methods well known to one skilled in the art.

### EXAMPLES

The present invention is further illustrated in the following Examples, in which parts and percentages are by weight and degrees are Celsius, unless otherwise stated. It should be understood that these Examples, while indicating embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

### Example 1

### Preparation of cDNA Libraries and Isolation and Sequencing of cDNA Clones

cDNA libraries may be prepared by any one of many methods available. For example, the cDNAs may be introduced into plasmid vectors by first preparing the cDNA libraries in UNI-ZAP™ XR vectors according to the manufacturer's protocol (Stratagene Cloning Systems, La Jolla, Calif.). The UNI-ZAP™ XR libraries are converted into plasmid libraries according to the protocol provided by Stratagene. Upon conversion, cDNA inserts will be contained in the plasmid vector pBLUESCRIPT®. In addition, the cDNAs may be introduced directly into precut BLUESCRIPT® II SK(+) vectors (Stratagene) using T4 DNA ligase (New England Biolabs), followed by transfection into DH10B cells according to the manufacturer's protocol (GIBCO BRL Products). Once the cDNA inserts are in plasmid vectors, plasmid DNAs are prepared from randomly picked bacterial colonies containing recombinant pBLUESCRIPT® plasmids, or the insert cDNA sequences are amplified via polymerase chain reaction using primers specific for vector sequences flanking the inserted cDNA sequences. Amplified insert DNAs or plasmid DNAs are sequenced in dye-primer sequencing reactions to generate partial cDNA sequences (expressed sequence tags or “ESTs”; see Adams et al., (1991) Science 252:1651-1656). The resulting ESTs are analyzed using a Perkin Elmer Model 377 fluorescent sequencer.

Full-insert sequence (FIS) data is generated utilizing a modified transposition protocol. Clones identified for FIS are recovered from archived glycerol stocks as single colonies, and plasmid DNAs are isolated via alkaline lysis. Isolated DNA templates are reacted with vector primed M13 forward and reverse oligonucleotides in a PCR-based sequencing reaction and loaded onto automated sequencers. Confirmation of clone identification is performed by sequence alignment to the original EST sequence from which the FIS request is made.

Confirmed templates are transposed via the Primer Island transposition kit (PE Applied Biosystems, Foster City, Calif.) which is based upon the Saccharomyces cerevisiae Ty1 transposable element (Devine and Boeke (1994) Nucleic Acids Res. 22:3765-3772). The in vitro transposition system places unique binding sites randomly throughout a population of large DNA molecules. The transposed DNA is then used to transform DH10B electro-competent cells (Gibco BRL/Life Technologies, Rockville, Md.) via electroporation. The transposable element contains an additional selectable marker (named DHFR; Fling and Richards (1983) Nucleic Acids Res. 11:5147-5158), allowing for dual selection on agar plates of only those subclones containing the integrated transposition. Multiple subclones are randomly selected from each transposition reaction, plasmid DNAs are prepared via alkaline lysis, and templates are sequenced (ABI PRISM® dye-terminator ReadyReaction mix) outward from the transposition event site, utilizing unique primers specific to the binding sites within the transposon.

Sequence data is collected (ABI PRISM® Collections) and assembled using Phred and Phrap (Ewing et al. (1998) Genome Res. 8:175-185; Ewing and Green (1998) Genome Res. 8:186-194). Phred is a public domain software program which re-reads the ABI sequence data, re-calls the bases, assigns quality values, and writes the base calls and quality values into editable output files. The Phrap sequence assembly program uses these quality values to increase the accuracy of the assembled sequence contigs. Assemblies are viewed by the Consed sequence editor (Gordon at al. (1998) Genome Res. 8:195-202).

In some of the clones the cDNA fragment may correspond to a portion of the 3′-terminus of the gene and does not cover the entire open reading frame. In order to obtain the upstream information one of two different protocols is used. The first of these methods results in the production of a fragment of DNA containing a portion of the desired gene sequence while the second method results in the production of a fragment containing the entire open reading frame. Both of these methods use two rounds of PCR amplification to obtain fragments from one or more libraries. The libraries some times are chosen based on previous knowledge that the specific gene should be found in a certain tissue and some times are randomly-chosen. Reactions to obtain the same gene may be performed on several libraries in parallel or on a pool of libraries. Library pools are normally prepared using from 3 to 5 different libraries and normalized to a uniform dilution. In the first round of amplification both methods use a vector-specific (forward) primer corresponding to a portion of the vector located at the 5′-terminus of the clone coupled with a gene-specific (reverse) primer. The first method uses a sequence that is complementary to a portion of the already known gene sequence while the second method uses a gene-specific primer complementary to a portion of the 3′-untranslated region (also referred to as UTR). In the second round of amplification a nested set of primers is used for both methods. The resulting DNA fragment is ligated into a pBLUESCRIPT® vector using a commercial kit and following the manufacturer's protocol. This kit is selected from many available from several vendors including INVITROGEN™ (Carlsbad, Calif.), Prornega Biotech (Madison, Wis.), and Gibco-BRL (Gaithersburg, Md.). The plasmid DNA is isolated by alkaline lysis method and submitted for sequencing and assembly using Phred/Phrap, as above.

### Example 2

### Identification of cDNA Clones

cDNA clones encoding GSH1 polypeptides can be identified by conducting BLAST (Basic Local Alignment Search Tool; Altschul et al. (1993) J. Biol. 215:403-410; see also the explanation of the BLAST algorithm on the world wide web site for the National Center for Biotechnology Information at the National Library of Medicine of the National Institutes of Health) searches for similarity to amino acid sequences contained in the BLAST “nr” database (comprising all non-redundant GenBank CDS translations, sequences derived from the 3-dimensional structure Brookhaven Protein Data Bank, the last major release of the SWISS-PROT protein sequence database, EMBL, and DDBJ databases). The DNA sequences from clones can be translated in all reading frames and compared for similarity to all publicly available protein sequences contained in the “nr” database using the BLASTX algorithm (Gish and States (1993) Nat. Genet. 3:266-272) provided by the NCBI. The polypeptides encoded by the cDNA sequences can be analyzed for similarity to all publicly available amino acid sequences contained in the “nr” database using the BLASTP algorithm provided by the National Center for Biotechnology Information (NCBI). For convenience, the P-value (probability) or the E-value (expectation) of observing a match of a cDNA-encoded sequence to a sequence contained in the searched databases merely by chance as calculated by BLAST are reported herein as “pLog” values, which represent the negative of the logarithm of the reported P-value or E-value. Accordingly, the greater the pLog value, the greater the likelihood that the cDNA-encoded sequence and the BLAST “hit” represent homologous proteins.

ESTs sequences can be compared to the Genbank database as described above. ESTs that contain sequences more 5- or 3-prime can be found by using the BLASTn algorithm (Altschul et al (1997) Nucleic Acids Res. 25:3389-3402.) against the Du Pont proprietary database comparing nucleotide sequences that share common or overlapping regions of sequence homology. Where common or overlapping sequences exist between two or more nucleic acid fragments, the sequences can be assembled into a single contiguous nucleotide sequence, thus extending the original fragment in either the 5 or 3 prime direction. Once the most 5-prime EST is identified, its complete sequence can be determined by Full Insert Sequencing as described above. Homologous genes belonging to different species can be found by comparing the amino acid sequence of a known gene (from either a proprietary source or a public database) against an EST database using the tBLASTn algorithm. The tBLASTn algorithm searches an amino acid query against a nucleotide database that is translated in all 6 reading frames. This search allows for differences in nucleotide codon usage between different species, and for codon degeneracy.

### Example 3

### Characterization of cDNA Clones

### Encoding GSH1 Polypeptides

cDNA libraries representing mRNAs from various tissues of maize, soybean and sunflower were prepared and cDNA clones encoding GSH1 polypeptides were identified. The characteristics of the cDNA libraries are described below.

TABLE 1
cDNA Libraries from Maize, Soybean and Sunflower

| Library | Description | Clone |
|---|---|---|
| sr1 | Soybean (Glycine max L.) root library | sr1.pk0076.f7 |
| | Soybean (Glycine max L.) two week old | |
| sl2 | developing seedlings treated with 2.5 ppm | sl2.pk0035.d12 |
| | chlorimuron | |
| ssl | Soybean (Glycine max L.) seedling 5-10 day | ssl.pk0035.b9 |
| — | Contig assembled from 19 maize sequences | PCO664734 |
| — | Contig assembled from 44 maize sequences | PCO664735 |
| hss1c | Sclerotinia infected sunflower plants | hss1c.pk021.l4 |
| hls1c | Sclerotinia infected sunflower plants | hls1c.pk008.e8 |
| hso1c | oxalate oxidase-transgenic sunflower plants | hso1c.pk021.k15 |

The BLAST search using the sequences from clones listed in Table 1 revealed similarity of the polypeptides encoded by the cDNAs to the GSH1 polypeptides from various organisms. As shown in Table 2 and FIGS. 1A-1E, certain cDNAs encoded polypeptides similar to GSH1 polypeptides from Arabidopsis (NCBI GI No. 1742963; SEQ ID NO:20), Phaseolus vulgaris (NCBI GI No, 6651029; SEQ ID NO:23), maize (NCBI GI No. 162464176; SEQ ID NO:24), Zinnia violacea (NCBI GI No. 50058088; SEQ ID NO:25), soybean (US Patent Publication No. US20040031072; SEQ ID NO:26) and rice (Japanese Patent Publication No. JP2005185101; SEQ ID NO:27). The published maize GSH1 polypeptide (SEQ ID NO:24; Gomez et al, 2004, Plant Physiol. 134:1662-1671) is lacking sixty-five N-terminal amino acids relative to a full-length precursor polypeptide that is targeted to the chloroplast (SEQ ID NO:8).

Shown in Tables 2 and 4 (non-patent literature) and Tables 3 and 5 (patent literature) are the BLASTP results for GSH1 precursor polypeptides (Tables 2 and 3) or GSH1 mature polypeptides (Tables 4 and 5). Also shown in Tables 2-5 are the percent sequence identity values for each pair of amino acid sequences using the Clustal V method of alignment with default parameters:

TABLE 2
Non-Patent Literature BLASTP Results for
GSH1 Precursor Polypeptides

| | | | BLASTP | Percent |
|---|---|---|---|---|
| | | Reference | pLog of | Sequence |
| Sequence | Plant | (SEQ ID NO) | E-value | Identity |
| SEQ ID NO: 2 | Soybean | GI No. 6651029 | >180 | 90.3% |
| | | (SEQ ID NO: 23) | | |
| SEQ ID NO: 8 | Corn | GI No. 162464176 | >180 | 100% |
| | | (SEQ ID NO: 24) | | |
| SEQ ID NO: | Corn | GI No. 162464176 | >180 | 96.6% |
| 12 | | (SEQ ID NO: 24) | | |
| SEQ ID NO: | Corn | GI No. 162464176 | >180 | 98.6% |
| 30 | | (SEQ ID NO: 24) | | |
| SEQ ID NO: | Sunflower | GI No. 50058088 | >180 | 93.3% |
| 16 | | (SEQ ID NO: 25) | | |

TABLE 3
Patent Literature BLASTP Results for
GSH1 Precursor Polypeptides

| | | | BLASTP | Percent |
|---|---|---|---|---|
| | | Reference | pLog of | Sequence |
| Sequence | Plant | (SEQ ID NO) | E-value | Identity |
| SEQ ID NO: 2 | Soybean | SEQ ID NO: | >180 | 96.0 |
| | | 252666 of | | |
| | | US20040031072 | | |
| | | (SEQ ID NO: 26) | | |
| SEQ ID NO: 8 | Corn | SEQ ID NO: 56195 | >180 | 89.8 |
| | | of JP2005185101 | | |
| | | (SEQ ID NO: 27) | | |
| SEQ ID NO: | Corn | SEQ ID NO: 56195 | >180 | 88.2% |
| 12 | | of JP2005185101 | | |
| | | (SEQ ID NO: 27) | | |
| SEQ ID NO: | Corn | SEQ ID NO: 56195 | >180 | 90.7% |
| 30 | | of JP2005185101 | | |
| | | (SEQ ID NO: 27) | | |
| SEQ ID NO: | Sunflower | SEQ ID NO: 2265 | >180 | 76.4% |
| 16 | | of WO2002010210 | | |
| | | (SEQ ID NO: 28) | | |

TABLE 4
Non-Patent Literature BLASTP Results for
GSH1 Mature Polypeptides

| | | | BLASTP | Percent |
|---|---|---|---|---|
| | | Reference | pLog of | Sequence |
| Sequence | Plant | (SEQ ID NO) | E-value | Identity |
| SEQ ID NO: 4 | Soybean | GI No. 6651029 | >180 | 96.2% |
| | | (SEQ ID NO: 23) | | |
| SEQ ID NO: | Corn | GI No. 162464176 | >180 | 100% |
| 10 | | (SEQ ID NO: 24) | | |
| SEQ ID NO: | Corn | GI No. 162464176 | >180 | 96.6% |
| 14 | | (SEQ ID NO: 24) | | |
| SEQ ID NO: | Corn | GI No. 162464176 | >180 | 98.6% |
| 32 | | (SEQ ID NO: 24) | | |
| SEQ ID NO: | Sunflower | GI No. 50058088 | >180 | 95.8% |
| 18 | | (SEQ ID NO: 25) | | |

TABLE 5
Patent Literature BLASTP Results for
GSH1 Mature Polypeptides

| | | | BLASTP | Percent |
|---|---|---|---|---|
| | | Reference | pLog of | Sequence |
| Sequence | Plant | (SEQ ID NO) | E-value | Identity |
| SEQ ID NO: 4 | Soybean | SEQ ID NO: | >180 | 95.3% |
| | | 252666 of | | |
| | | US20040031072 | | |
| | | (SEQ ID NO: 26) | | |
| SEQ ID NO: | Corn | SEQ ID NO: 56195 | >180 | 92.9% |
| 10 | | of JP2005185101 | | |
| | | (SEQ ID NO: 27) | | |
| SEQ ID NO: | Corn | SEQ ID NO: 56195 | >180 | 92.0% |
| 14 | | of JP2005185101 | | |
| | | (SEQ ID NO: 27) | | |
| SEQ ID NO: | Corn | SEQ ID NO: 56195 | >180 | 93.8% |
| 32 | | of JP2005185101 | | |
| | | (SEQ ID NO: 27) | | |
| SEQ ID NO: | Sunflower | SEQ ID NO: 2265 | >180 | 84.7% |
| 18 | | of WO2002010210 | | |
| | | (SEQ ID NO: 28) | | |

FIGS. 1A-1E present an alignment of the amino acid sequences of the GSH1 precursor polypeptides set forth in SEQ ID NOs:2, 8, 12, 30, 16, 20, 23, 25, 26, 27, 28 and the maize GSH1 polypeptide of SEQ ID NO:24 that lacks a transit peptide. FIG. 2 presents the percent sequence identities and divergence values for each sequence pair presented in FIGS. 1A-1E.

FIGS. 3A-3C present an alignment of the amino acid sequences of the GSH1 mature polypeptides set forth in SEQ ID NOs:4, 10, 14, 32, 18, 22 and the maize GSH1 polypeptide of SEQ ID NO:24 that lacks a transit peptide. FIG. 4 presents the percent sequence identities and divergence values for each sequence pair presented in FIGS. 3A-3C.

Sequence alignments and percent identity calculations were performed using the MEGALIGN® program of the LASERGENE® bioinformatics computing suite (DNASTAR® Inc., Madison, Wis.). Multiple alignment of the sequences was performed using the Clustal V method of alignment (Higgins and Sharp (1989) CABIOS. 5:151-153) with the default parameters (GAP PENALTY=10, GAP LENGTH PENALTY=10), Default parameters for pairwise alignments using the Clustal method were KTUPLE=1, GAP PENALTY=3, WINDOW=5 and DIAGONALS SAVED=5.

A soybean cDNA clone, ssl.pk0035.b9, was identified that encodes a full-length soybean precursor GSH1 polypeptide, designated “GM-GSH1b”. Primers PHN_{—}131845 (SEQ ID NO:39) and PHN_{—}131846 (SEQ ID NO:40) were designed and a PCR product was amplified from clone ssl.pk0035.b9 (SEQ ID NO:41). The nucleotide sequence of the protein-coding region for the precursor GM-GSH1b polypeptide is presented in SEQ ID NO:42. The corresponding amino acid sequence is presented in SEQ ID NO:43. The amino acid sequence of SEQ ID NO:43 differs from that of SEQ ID NO:2 by a single amino acid; there is a R-to-K change at amino acid position 249 (R in SEQ ID NO:2; K in SEQ ID NO:43.

Sequence alignments and BLAST scores and probabilities indicate that the nucleic acid fragments comprising the instant cDNA clones encode GSH1 polypeptides.

### Example 4

### Preparation of a Plant Expression Vector

### Containing a GSH1 Polypeptide Gene

Sequences homologous to the GSH1 polypeptide encoded by Arabidopsis can be identified using sequence comparison algorithms such as BLAST (Basic Local Alignment Search Tool; Altschul et al., J. Mol. Biol. 215:403-410 (1993); see also the explanation of the BLAST algorithm on the world wide web site for the National Center for Biotechnology Information at the National Library of Medicine of the National Institutes of Health). Sequences encoding homologous lead gene polypeptides can be PCR-amplified by either of the following methods.

Method 1 (RNA-based): If the 5′ and 3′ sequence information for the protein-coding region of a gene encoding a GSH1 polypeptide is available, gene-specific primers can be designed. RT-PCR can be used with plant RNA to obtain a nucleic acid fragment containing the protein-coding region flanked by attB1 (SEQ ID NO:33) and attB2 (SEQ ID NO:34) sequences. The primer may contain a consensus Kozak sequence (CAACA) upstream of the start codon.

Method 2 (DNA-based): Alternatively, if a cDNA clone is available for a gene encoding a homolog to a GSH1 polypeptide, the entire cDNA insert (containing 5′ and 3′ non-coding regions) can be PCR amplified, Forward and reverse primers can be designed that contain either the attB1 sequence and vector-specific sequence that precedes the cDNA insert or the attB2 sequence and vector-specific sequence that follows the cDNA insert, respectively. For a cDNA insert cloned into the vector pBulescript SK+, the forward primer VC062 (SEC) ID NO:35) and the reverse primer VC063 (SEQ ID NO:36) can be used.

Methods 1 and 2 can be modified according to procedures known by one skilled in the art. For example, the primers of Method 1 may contain restriction sites instead of attB1 and attB2 sites, for subsequent cloning of the FOR product into a vector containing attB1 and attB2 sites. Additionally, Method 2 can involve amplification from a cDNA clone, a lambda clone, a BAC clone or genomic DNA.

A PCR product obtained by either method above can be combined with the GATEWAY® donor vector, such as pDONR™/Zeo (INVITROGEN™) or pDONR™221 (INVITROGEN™), using a BP Recombination Reaction. This process removes the bacteria lethal ccdB gene, as well as the chloramphenicol resistance gene (CAM) from pDONR™221 and directionally clones the PCR product with flanking attB1 and attB2 sites to create an entry clone. Using the INVITROGEN™ GATEWAY® CLONASE™ technology, the sequence from the entry clone encoding the homologous lead gene polypeptide can then be transferred to a suitable destination vector to obtain a plant expression vector for transformation.

Alternatively a MultiSite GATEWAY® LR recombination reaction between multiple entry clones and a suitable destination vector can be performed to create an expression vector.

### Example 5

### Transformation of Soybean

Soybean plants can be transformed to overexpress an Arabidopsis GSH1 polypeptide gene or the corresponding homologs from various species in order to examine the resulting phenotype.

To induce somatic embryos, cotyledons, 3-5 mm in length dissected from surface sterilized, immature seeds of the soybean cultivar A2872, can be cultured in the light or dark at 26° C. on an appropriate agar medium for 6-10 weeks. Somatic embryos, which produce secondary embryos, are then excised and placed into a suitable liquid medium. After repeated selection for clusters of somatic embryos which multiply as early, globular staged embryos, the suspensions are maintained as described below.

Soybean embryogenic suspension cultures can be maintained in 35 mL liquid media on a rotary shaker, 150 rpm, at 26° C. with florescent lights on a 16:8 hour day/night schedule. Cultures are subcultured every two weeks by inoculating approximately 35 mg of tissue into 35 mL of liquid medium. Soybean embryogenic suspension cultures may then be transformed by the method of particle gun bombardment (Klein et al. (1987) Nature (London) 327:70-73, U.S. Pat. No. 4,945,050). A DUPONT™ BIOLISTIC™ PDS1000/HE instrument (helium retrofit) can be used for these transformations.

A selectable marker gene which can be used to facilitate soybean transformation is a chimeric gene composed of the 35S promoter from cauliflower mosaic virus (Odell et al. (1985) Nature 313:810-812), the hygromycin phosphotransferase gene from plasmid pJR225 (from E. coil; Gritz et al. (1983) Gene 25:179-188) and the 3 region of the nopaline synthase gene from the T-DNA of the Ti plasmid of Agrobacterium tumefaciens. Another selectable marker gene which can be used to facilitate soybean transformation is an herbicide-resistant acetolactate synthase (ALS) gene from soybean or Arabidopsis. ALS is the first common enzyme in the biosynthesis of the branched-chain amino acids valine, leucine and isoleucine. Mutations in ALS have been identified that convey resistance to some or all of three classes of inhibitors of ALS (U.S. Pat. No. 5,013,659; the entire contents of which are herein incorporated by reference). Expression of the herbicide-resistant ALS gene can be under the control of a SAM synthetase promoter (U.S. patent application No. US-2003-0226166-A1; the entire contents of which are herein incorporated by reference).

To 50 μL of a 60 mg/mL 1 μm gold particle suspension is added (in order): 5 μL DNA (1 μg/μL), 20 μL spermidine (0.1 M), and 50 μL CaCl₂(2.5 M). The particle preparation is then agitated for three minutes, spun in a microfuge for 10 seconds and the supernatant removed. The DNA-coated particles are then washed once in 400 μL 70% ethanol and resuspended in 40 μL of anhydrous ethanol. The DNA/particle suspension can be sonicated three times for one second each. Five μL of the DNA-coated gold particles are then loaded on each macro carrier disk.

Approximately 300-400 mg of a two-week-old suspension culture is placed in an empty 60×15 mm petri dish and the residual liquid removed from the tissue with a pipette. For each transformation experiment, approximately 5-10 plates of tissue are normally bombarded. Membrane rupture pressure is set at 1100 psi and the chamber is evacuated to a vacuum of 28 inches mercury. The tissue is placed approximately 3.5 inches away from the retaining screen and bombarded three times. Following bombardment, the tissue can be divided in half and placed back into liquid and cultured as described above.

Five to seven days post bombardment, the liquid media may be exchanged with fresh media, and eleven to twelve days post bombardment with fresh media containing 50 mg/mL hygromycin. This selective media can be refreshed weekly. Seven to eight weeks post bombardment, green, transformed tissue may be observed growing from untransformed, necrotic embryogenic clusters. Isolated green tissue is removed and inoculated into individual flasks to generate new, clonally propagated, transformed embryogenic suspension cultures. Each new line may be treated as an independent transformation event. These suspensions can then be subcultured and maintained as clusters of immature embryos or regenerated into whole plants by maturation and germination of individual somatic embryos.

### Example 6

### Transformation of Maize Using Particle Bombardment

Maize plants can be transformed to overexpress an Arabidopsis GSH1 polypeptide gene or the corresponding homologs from various species in order to examine the resulting phenotype.

Expression of the gene in a maize transformation vector can be under control of a constitutive promoter such as the maize ubiquitin promoter (Christensen et al., (1989) Plant Mol. Biol. 12:619-632 and Christensen et al., (1992) Plant Mol. Biol. 18:675-689)

The recombinant DNA construct can be introduced into corn cells by the following procedure. Immature corn embryos can be dissected from developing caryopses derived from crosses of the inbred corn lines H99 and LH132. The embryos are isolated 10 to 11 days after pollination when they are 1.0 to 1.5 mm long. The embryos are then placed with the axis-side facing down and in contact with agarose-solidified N6 medium (Chu et al. (1975)Sci. Sin. Peking 18:659-668). The embryos are kept in the dark at 27° C. Friable embryogenic callus consisting of undifferentiated masses of cells with somatic proembryoids and embryoids borne on suspensor structures proliferates from the scutellum of these immature embryos. The embryogenic callus isolated from the primary explant can be cultured on N6 medium and sub-cultured on this medium every 2 to 3 weeks.

The plasmid, p35S/Ac (obtained from Dr. Peter Eckes, Hoechst Ag, Frankfurt, Germany) may be used in transformation experiments in order to provide for a selectable marker. This plasmid contains the Pat gene (see European Patent Publication 0 242 236) which encodes phosphinothricin acetyl transferase (PAT). The enzyme PAT confers resistance to herbicidal glutamine synthetase inhibitors such as phosphinothricin. The pat gene in p35S/Ac is under the control of the 35S promoter from cauliflower mosaic virus (Odell et al. (1985) Nature 313:810-812) and the 3′ region of the nopaline synthase gene from the T-DNA of the Ti plasmid of Agrobacterium tumefaciens.

The particle bombardment method (Klein et al. (1987) Nature 327:70-73) may be used to transfer genes to the callus culture cells. According to this method, gold particles (1 μm in diameter) are coated with DNA using the following technique. Ten μg of plasmid DNAs are added to 50 μL of a suspension of gold particles (60 mg per mL). Calcium chloride (50 μL of a 2.5 M solution) and spermidine free base (20 μL of a 1.0 M solution) are added to the particles. The suspension is vortexed during the addition of these solutions. After 10 minutes, the tubes are briefly centrifuged (5 sec at 15,000 rpm) and the supernatant removed. The particles are resuspended in 200 μL of absolute ethanol, centrifuged again and the supernatant removed. The ethanol rinse is performed again and the particles resuspended in a final volume of 30 μL of ethanol. An aliquot (5 μL) of the DNA-coated gold particles can be placed in the center of a KAPTON™ flying disc (Bio-Rad Labs). The particles are then accelerated into the corn tissue with a DUPONT™ BIOLISTIC™ PDS-1000/He (Bio-Rad Instruments, Hercules Calif.), using a helium pressure of 1000 psi, a gap distance of 0.5 cm and a flying distance of 1.0 cm.

For bombardment, the embryogenic tissue is placed on filter paper over agarose-solidified N6 medium. The tissue is arranged as a thin lawn and covers a circular area of about 5 cm in diameter. The petri dish containing the tissue can be placed in the chamber of the PDS-1000/He approximately 8 cm from the stopping screen. The air in the chamber is then evacuated to a vacuum of 28 inches of Hg. The macrocarrier is accelerated with a helium shock wave using a rupture membrane that bursts when the He pressure in the shock tube reaches 1000 psi.

Seven days after bombardment the tissue can be transferred to N6 medium that contains bialaphos (5 mg per liter) and lacks casein or praline. The tissue continues to grow slowly on this medium. After an additional 2 weeks the tissue can be transferred to fresh N6 medium containing bialaphos. After 6 weeks, areas of about 1 cm in diameter of actively growing callus can be identified on some of the plates containing the bialaphos-supplemented medium. These calli may continue to grow when sub-cultured on the selective medium.

Plants can be regenerated from the transgenic callus by first transferring dusters of tissue to N6 medium supplemented with 0.2 mg per liter of 2,4-D. After two weeks the tissue can be transferred to regeneration medium (Fromm et al. (1990) Bio/Technology 8:833-839). Transgenic T0 plants can be regenerated and their phenotype determined following high throughput (“HTP”) procedures. T1 seed can be collected.

### Example 7

Electroporation of Agrobacterium tumefaciens LBA4404

Electroporation competent cells (40 μL), such as Agrobacterium tumefaciens LBA4404 containing a superbinary vir plasmid PHP10523 (pSB1; U.S. Pat. No. 5,731,179A; Komari et al., 1996, Plant J. 10:165-174), are thawed on ice (20-30 min). PHP10523 contains VIR genes for T-DNA transfer, an Agrobacterium low copy number plasmid origin of replication, a tetracycline resistance gene, and a Cos site for in vivo DNA bimolecular recombination. Meanwhile the electroporation cuvette is chilled on ice. The electroporator settings are adjusted to 2.1 kV. A DNA aliquot (0.5 μL parental DNA at a concentration of 0.2 μg-1.0 μg in low salt buffer or twice distilled H₂O) is mixed with the thawed Agrobacterium tumefaciens LBA4404 cells while still on ice. The mixture is transferred to the bottom of electroporation cuvette and kept at rest on ice for 1-2 min. The cells are electroporated (Eppendorf electroporator 2510) by pushing the “pulse” button twice (ideally achieving a 4.0 millisecond pulse). Subsequently, 0.5 mL of room temperature 2xYT medium (or SOC medium) are added to the cuvette and transferred to a 15 mL snap-cap tube (e.g., FALCON™ tube). The cells are incubated at 28-30° C., 200-250 rpm for 3 h.

Aliquots of 250 μL are spread onto plates containing YM medium and 50 μg/mL spectinomycin and incubated three days at 28-30° C. To increase the number of transformants one of two optional steps can be performed:

Option 1: Overlay plates with 30 μL of 15 mg/mL rifampicin. LBA4404 has a chromosomal resistance gene for rifampicin. This additional selection eliminates some contaminating colonies observed when using poorer preparations of LBA4404 competent cells.

Option 2: Perform two replicates of the electroporation to compensate for poorer electrocompetent cells.

Identification of Transformants:

Four independent colonies are picked and streaked on plates containing AB minimal medium and 50 μg/mL spectinomycin for isolation of single colonies. The plates are incubated at 28° C. for two to three days. A single colony for each putative co-integrate is picked and inoculated with 4 mL of 10 g/L bactopeptone, 10 g/L yeast extract, 5 g/L sodium chloride and 50 mg/L spectinomycin. The mixture is incubated for 24 h at 28° C. with shaking. Plasmid DNA from 4 mL of culture is isolated using QIAGEN® Miniprep and an optional Buffer PB wash. The DNA is eluted in 30 μL. Aliquots of 2 μL are used to electroporate 20 μL of DH10b+20 μL of twice distilled H₂O as per above. Optionally a 15 μL aliquot can be used to transform 75-100 μL of INVITROGEN™ Library Efficiency DH5α. The cells are spread on plates containing LB medium and 50 μg/mL spectinomycin and incubated at 37° C. overnight.

Three to four independent colonies are picked for each putative co-integrate and inoculated 4 mL of 2xYT medium (10 g/L bactopeptone, 10 g/L yeast extract, 5 g/L sodium chloride) with 50 μg/mL spectinomycin. The cells are incubated at 37° C. overnight with shaking. Next, isolate the plasmid DNA from 4 mL of culture using QIAPREP® Miniprep with optional Buffer PB wash (elute in 50 μL). Use 8 μL for digestion with Sall (using parental DNA and PHP10523 as controls). Three more digestions using restriction enzymes BamHI, EcoRI, and HindIII are performed for 4 plasmids that represent 2 putative co-integrates with correct Sall digestion pattern (using parental DNA and PHP10523 as controls). Electronic gels are recommended for comparison.

### Example 8

### Transformation of Maize Using Agrobacterium

Maize plants can be transformed to overexpress an Arabidopsis GSH1 polypeptide gene or the corresponding homologs from various species in order to examine the resulting phenotype.

Agrobacterium-mediated transformation of maize is performed essentially as described by Zhao et al. in Meth. Mot. Biol. 318:315-323 (2006) (see also Zhao et al., Mol. Breed. 8:323-333 (2001) and U.S. Pat. No. 5,981,840 issued Nov. 9, 1999, incorporated herein by reference). The transformation process involves bacterium innoculation, co-cultivation, resting, selection and plant regeneration.

1. Immature Embryo Preparation:

Immature maize embryos are dissected from caryopses and placed in a 2 mL microtube containing 2 mL PHI-A medium.

2. Agrobacterium Infection and Co-Cultivation of Immature Embryos:

2.1 infection Step:

PHI-A medium of (1) is removed with 1 mL micropipettor, and 1 mL of Agrobacterium suspension is added. The tube is gently inverted to mix. The mixture is incubated for 5 min at room temperature.

2.2 Co-Culture Step:

The Agrobacterium suspension is removed from the infection step with a 1 mL micropipettor. Using a sterile spatula the embryos are scraped from the tube and transferred to a plate of PHI-B medium in a 100×15 mm Petri dish. The embryos are oriented with the embryonic axis down on the surface of the medium. Plates with the embryos are cultured at 20° C., in darkness, for three days, L-Cysteine can be used in the co-cultivation phase. With the standard binary vector, the co-cultivation medium supplied with 100-400 mg/L L-cysteine is critical for recovering stable transgenic events.

3. Selection of Putative Transgenic Events:

To each plate of PHI-D medium in a 100×15 mm Petri dish, 10 embryos are transferred, maintaining orientation and the dishes are sealed with parafilm. The plates are incubated in darkness at 28° C. Actively growing putative events, as pale yellow embryonic tissue, are expected to be visible in six to eight weeks. Embryos that produce no events may be brown and necrotic, and lithe friable tissue growth is evident. Putative transgenic embryonic tissue is subcultured to fresh PHI-plates at two-three week intervals, depending on growth rate. The events are recorded.

4. Regeneration of T0 Plants:

Embryonic tissue propagated on PHI-D medium is subcultured to PHI-E medium (somatic embryo maturation medium), in 100×25 mm Petri dishes and incubated at 28° C., in darkness, until somatic embryos mature, for about ten to eighteen days. Individual, matured somatic embryos with well-defined scutellum and coleoptile are transferred to PHI-F embryo germination medium and incubated at 28° C. in the light (about 80 μE from cool white or equivalent fluorescent lamps). In seven to ten days, regenerated plants, about 10 cm tall, are potted in horticultural mix and hardened-off using standard horticultural methods.

Media for Plant Transformation:
1. PHI-A: 4 g/L CHU basal salts, 1.0 mL/L 1000×Eriksson's vitamin mix, 0.5 mg/L thiamin HCl, 1.5 mg/L 2,4-D, 0.69 g/L L-proline, 68.5 g/L sucrose, 36 g/L glucose, pH 5.2. Add 100 μM acetosyringone (filter-sterilized).
2. PHI-B: PHI-A without glucose, increase 2,4-D to 2 mg/L, reduce sucrose to 30 g/L and supplemente with 0.85 mg/L silver nitrate (filter-sterilized), 3.0 g/L GELRITE®, 100 μM acetosyringone (filter-sterilized), pH 5.8.
3. PHI-C: PHI-B without GELRITE® and acetosyringonee, reduce 2,4-D to 1.5 mg/L and supplemente with 8.0 g/L agar, 0.5 g/L 2-[N-morpholino]ethane-sulfonic acid (MES) buffer, 100 mg/L carbenicillin (filter-sterilized).
4. PHI-D: PHI-C supplemented with 3 mg/L bialaphos (filter-sterilized).
5. PHI-E: 4.3 g/L of Murashige and Skoog (MS) salts, (Gibco, BRL 11117-074), 0.5 mg/L nicotinic acid, 0.1 mg/L thiamine HCl, 0.5 mg/L pyridoxine HCl, 2.0 mg/L glycine, 0.1 g/L myo-inositol, 0.5 mg/L zeatin (Sigma, Cat. No. Z-0164), 1 mg/L indole acetic acid (IAA), 26.4 μg/L abscisic acid (ABA), 60 g/L sucrose, 3 mg/L bialaphos (filter-sterilized), 100 mg/L carbenicillin (filter-sterilized), 8 g/L agar, pH 5.6.
6. PHI-E without zeatin, IAA, ABA; reduce sucrose to 40 g/L; replacing agar with 1.5 g/L GELRITE®; pH 5.6.

Plants can be regenerated from the transgenic callus by first transferring dusters of tissue to N6 medium supplemented with 0.2 mg per liter of 2,4-D. After two weeks the tissue can be transferred to regeneration medium (Fromm et al., Bio/Technology 8:833-839 (1990)).

Transgenic T0 plants can be regenerated and their phenotype determined. T1 seed can be collected.

Furthermore, a recombinant DNA construct containing a GSH1 polypeptide gene can be introduced into an elite maize inbred line either by direct transformation or introgression from a separately transformed line.

### Example 9

### Transformation of Gaspe Flint Derived Maize Lines

Maize plants can be transformed to overexpress the Arabidopsis GSH1 polypeptide gene or the corresponding homologs from other species in order to examine the resulting phenotype.

Recipient Plants:

Recipient plant cells can be from a uniform maize line having a short life cycle (“fast cycling”), a reduced size, and high transformation potential. Typical of these plant cells for maize are plant cells from any of the publicly available Gaspe Flint (GBF) line varieties. One possible candidate plant line variety is the F1 hybrid of GBF×QTM (Quick Turnaround Maize, a publicly available form of Gaspe Flint selected for growth under greenhouse conditions) disclosed in Tomes et al. U.S. Patent Application Publication No. 2003/0221212. Transgenic plants obtained from this line are of such a reduced size that they can be grown in four inch pots (¼ the space needed for a normal sized maize plant) and mature in less than 2.5 months. (Traditionally 3.5 months is required to obtain transgenic T0 seed once the transgenic plants are acclimated to the greenhouse.) Another suitable line is a double haploid line of GS3 (a highly transformable line) X Gaspe Hint. Yet another suitable line is a transformable elite inbred line carrying a transgene which causes early flowering, reduced stature, or both.

Transformation Protocol:

Any suitable method may be used to introduce the transgenes into the maize cells, including but not limited to inoculation type procedures using Agrobacterium based vectors. Transformation may be performed on immature embryos of the recipient (target) plant.

Precision Growth and Plant Tracking:

The event population of transgenic (T0) plants resulting from the transformed maize embryos is grown in a controlled greenhouse environment using a modified randomized block design to reduce or eliminate environmental error. A randomized block design is a plant layout in which the experimental plants are divided into groups (e.g., thirty plants per group), referred to as blocks, and each plant is randomly assigned a location with the block.

For a group of thirty plants, twenty-four transformed, experimental plants and six control plants (plants with a set phenotype) (collectively, a “replicate group”) are placed in pots which are arranged in an array (a.k.a. a replicate group or block) on a table located inside a greenhouse. Each plant, control or experimental, is randomly assigned to a location with the block which is mapped to a unique, physical greenhouse location as well as to the replicate group. Multiple replicate groups of thirty plants each may be grown in the same greenhouse in a single experiment. The layout (arrangement) of the replicate groups should be determined to minimize space requirements as well as environmental effects within the greenhouse. Such a layout may be referred to as a compressed greenhouse layout.

An alternative to the addition of a specific control group is to identify those transgenic plants that do not express the gene of interest. A variety of techniques such as RT-PCR can be applied to quantitatively assess the expression level of the introduced gene. T0 plants that do not express the transgene can be compared to those which do.

Each plant in the event population is identified and tracked throughout the evaluation process, and the data gathered from that plant is automatically associated with that plant so that the gathered data can be associated with the transgene carried by the plant. For example, each plant container can have a machine readable label (such as a Universal Product Code (UPC) bar code) which includes information about the plant identity, which in turn is correlated to a greenhouse location so that data obtained from the plant can be automatically associated with that plant.

Alternatively any efficient, machine readable, plant identification system can be used, such as two-dimensional matrix codes or even radio frequency identification tags (RFID) in which the data is received and interpreted by a radio frequency receiver/processor. See U.S. Published Patent Application No. 2004/0122592, incorporated herein by reference.

Phenotypic Analysis Using Three-Dimensional Imaging:

Each greenhouse plant in the T0 event population, including any control plants, is analyzed for agronomic characteristics of interest, and the agronomic data for each plant is recorded or stored in a manner so that it is associated with the identifying data (see above) for that plant. Confirmation of a phenotype (gene effect) can be accomplished in the T1 generation with a similar experimental design to that described above.

The T0 plants are analyzed at the phenotypic level using quantitative, non-destructive imaging technology throughout the plant's entire greenhouse life cycle to assess the traits of interest. For example, a digital imaging analyzer is used for automatic multi-dimensional analyzing of total plants. The imaging may be done inside the greenhouse. Two camera systems, located at the top and side, and an apparatus to rotate the plant, are used to view and image plants from all sides. Images are acquired from the top, front and side of each plant. All three images together provide sufficient information to evaluate the biomass, size and morphology of each plant.

Due to the change in size of the plants from the time the first leaf appears from the soil to the time the plants are at the end of their development, the early stages of plant development are best documented with a higher magnification from the top. This may be accomplished by using a motorized zoom lens system that is fully controlled by the imaging software.

In a single imaging analysis operation, the following events occur: (1) the plant is conveyed inside the analyzer area, rotated 360 degrees so its machine readable label can be read, and left at rest until its leaves stop moving; (2) the side image is taken and entered into a database; (3) the plant is rotated 90 degrees and again left at rest until its leaves stop moving, and (4) the plant is transported out of the analyzer.

Plants are allowed at least six hours of darkness per twenty four hour period in order to have a normal day/night cycle.

Imaging Instrumentation:

Any suitable imaging instrumentation may be used, including but not limited to light spectrum digital imaging instrumentation commercially available from LemnaTec GmbH of Wurselen, Germany. The images are taken and analyzed with a LemnaTec Scanalyzer HTS LT-0001-2 having a ½″ IT Progressive Scan IEE CCD imaging device. The imaging cameras may be equipped with a motor zoom, motor aperture and motor focus. All camera settings may be made using LemnaTec software. For example, the instrumental variance of the imaging analyzer is less than about 5% for major components and less than about 10% for minor components.

Software:

The imaging analysis system comprises a LemnaTec HTS Bonit software program for color and architecture analysis and a server database for storing data from about 500,000 analyses, including the analysis dates. The original images and the analyzed images are stored together to allow the user to do as much reanalyzing as desired. The database can be connected to the imaging hardware for automatic data collection and storage. A variety of commercially available software systems (e.g. Matlab, others) can be used for quantitative interpretation of the imaging data, and any of these software systems can be applied to the image data set.

Conveyor System:

A conveyor system with a plant rotating device may be used to transport the plants to the imaging area and rotate them during imaging. For example, up to four plants, each with a maximum height of 1.5 m, are loaded onto cars that travel over the circulating conveyor system and through the imaging measurement area. In this case the total footprint of the unit (imaging analyzer and conveyor loop) is about 5 m×5 m.

The conveyor system can be enlarged to accommodate more plants at a time. The plants are transported along the conveyor loop to the imaging area and are analyzed for up to 50 seconds per plant. Three views of the plant are taken. The conveyor system, as well as the imaging equipment, should be capable of being used in greenhouse environmental conditions.

Illumination:

Any suitable mode of illumination may be used for the image acquisition. For example, a top light above a black background can be used. Alternatively, a combination of top- and backlight using a white background can be used. The illuminated area should be housed to ensure constant illumination conditions. The housing should be longer than the measurement area so that constant light conditions prevail without requiring the opening and closing or doors. Alternatively, the illumination can be varied to cause excitation of either transgene (e.g., green fluorescent protein (GFP), red fluorescent protein (RFP)) or endogenous (e.g. Chlorophyll) fluorophores.

Biomass Estimation Based on Three-Dimensional Imaging:

For best estimation of biomass the plant images should be taken from at least three axes, for example, the top and two side (sides 1 and 2) views. These images are then analyzed to separate the plant from the background, pot and pollen control bag (if applicable). The volume of the plant can be estimated by the calculation:

Volume(voxels)=\sqrt{TopArea(pixels)}×\sqrt{Side1Area(pixels)}×\sqrt{Side2area(pixels)}

In the equation above the units of volume and area are “arbitrary units”. Arbitrary units are entirely sufficient to detect gene effects on plant size and growth in this system because what is desired is to detect differences (both positive-larger and negative-smaller) from the experimental mean, or control mean. The arbitrary units of size (e.g. area) may be trivially converted to physical measurements by the addition of a physical reference to the imaging process. For instance, a physical reference of known area can be included in both top and side imaging processes. Based on the area of these physical references a conversion factor can be determined to allow conversion from pixels to a unit of area such as square centimeters (cm²). The physical reference may or may not be an independent sample. For instance, the pot, with a known diameter and height, could serve as an adequate physical reference.

Color Classification:

The imaging technology may also be used to determine plant color and to assign plant colors to various color classes. The assignment of image colors to color classes is an inherent feature of the LemnaTec software. With other image analysis software systems color classification may be determined by a variety of computational approaches.

For the determination of plant size and growth parameters, a useful classification scheme is to define a simple color scheme including two or three shades of green and, in addition, a color class for chlorosis, necrosis and bleaching, should these conditions occur. A background color class which includes non plant colors in the image (for example pot and soil colors) is also used and these pixels are specifically excluded from the determination of size. The plants are analyzed under controlled constant illumination so that any change within one plant over time, or between plants or different batches of plants (e.g. seasonal differences) can be quantified.

In addition to its usefulness in determining plant size growth, color classification can be used to assess other yield component traits. For these other yield component traits additional color classification schemes may be used. For instance, the trait known as “staygreen”, which has been associated with improvements in yield, may be assessed by a color classification that separates shades of green from shades of yellow and brown (which are indicative of senescing tissues). By applying this color classification to images taken toward the end of the T0 or T1 plants' life cycle, plants that have increased amounts of green colors relative to yellow and brown colors (expressed, for instance, as Green/Yellow Ratio) may be identified. Plants with a significant difference in this Green/Yellow ratio can be identified as carrying transgenes which impact this important agronomic trait.

The skilled plant biologist will recognize that other plant colors arise which can indicate plant health or stress response (for instance anthocyanins), and that other color classification schemes can provide further measures of gene action in traits related to these responses.

Plant Architecture Analysis:

Transgenes which modify plant architecture parameters may also be identified using the present invention, including such parameters as maximum height and width, internodal distances, angle between leaves and stem, number of leaves starting at nodes and leaf length. The LemnaTec system software may be used to determine plant architecture as follows. The plant is reduced to its main geometric architecture in a first imaging step and then, based on this image, parameterized identification of the different architecture parameters can be performed. Transgenes that modify any of these architecture parameters either singly or in combination can be identified by applying the statistical approaches previously described.

Pollen Shed Date:

Pollen shed date is an important parameter to be analyzed in a transformed plant, and may be determined by the first appearance on the plant of an active male flower. To find the male flower object, the upper end of the stem is classified by color to detect yellow or violet anthers. This color classification analysis is then used to define an active flower, which in turn can be used to calculate pollen shed date.

Alternatively, pollen shed date and other easily visually detected plant attributes (e.g. pollination date, first silk date) can be recorded by the personnel responsible for performing plant care. To maximize data integrity and process efficiency this data is tracked by utilizing the same barcodes utilized by the LemnaTec light spectrum digital analyzing device. A computer with a barcode reader, a palm device, or a notebook PC may be used for ease of data capture recording time of observation, plant identifier, and the operator who captured the data.

Orientation of the Plants:

Mature maize plants grown at densities approximating commercial planting often have a planar architecture. That is, the plant has a clearly discernable broad side, and a narrow side. The image of the plant from the broadside is determined. To each plant a well defined basic orientation is assigned to obtain the maximum difference between the broadside and edgewise images. The top image is used to determine the main axis of the plant, and an additional rotating device is used to turn the plant to the appropriate orientation prior to starting the main image acquisition.

### Example 10

### Screening of Gaspe Flint Derived

### Maize Lines for Drought Tolerance

Transgenic Gaspe Flint derived maize lines containing the candidate gene can be screened for tolerance to drought stress in the following manner.

Transgenic maize plants are subjected to well-watered conditions (control) and to drought-stressed conditions. Transgenic maize plants are screened at the Ti stage or later.

For plant growth, the soil mixture consists of ⅓ TURFACE®, ⅓ SB3300 and ⅓ sand. All pots are filled with the same amount of soil±10 grams. Pots are brought up to 100% field capacity (“FC”) by hand watering. All plants are maintained at 60% FC using a 20-10-20 (N—P-K) 125 ppm N nutrient solution. Throughout the experiment pH is monitored at least three times weekly for each table. Starting at 13 days after planting (DAP), the experiment can be divided into two treatment groups, well watered and reduce watered. All plants comprising the reduced watered treatment are maintained at 40% FC while plants in the well watered treatment are maintained at 80% FC. Reduced watered plants are grown for 10 days under chronic drought stress conditions (40% FC). All plants are imaged daily throughout chronic stress period. Plants are sampled for metabolic profiling analyses at the end of chronic drought period, 22 DAP. At the conclusion of the chronic stress period all plants are imaged and measured for chlorophyll fluorescence. Reduced watered plants are subjected to a severe drought stress period followed by a recovery period, 23-31 DAP and 32-34 DAP respectively. During the severe drought stress, water and nutrients are withheld until the plants reached 8% FC. At the conclusion of severe stress and recovery periods all plants are again imaged and measured for chlorophyll fluorescence. The probability of a greater Student's t Test is calculated for each transgenic mean compared to the appropriate null mean (either segregant null or construct null). A minimum (P<t) of 0.1 is used as a cut off for a statistically significant result.

### Example 11

### Yield Analysis of Maize Lines Containing

### Genes Encoding GSH1 Polypeptides

A recombinant DNA construct containing a GSH1 polypeptide gene can be introduced into an elite maize inbred line either by direct transformation or introgression from a separately transformed line.

Transgenic plants, either inbred or hybrid, can undergo more vigorous field-based experiments to study yield enhancement and/or stability under well-watered and water-limiting conditions.

Subsequent yield analysis can be done to determine whether plants that contain the validated Arabidopsis lead gene have an improvement in yield performance under water-limiting conditions, when compared to the control plants that do not contain the validated Arabidopsis lead gene. Specifically, drought conditions can be imposed during the flowering and/or grain fill period for plants that contain the validated Arabidopsis lead gene and the control plants. Reduction in yield can be measured for both. Plants containing the validated Arabidopsis lead gene have less yield loss relative to the control plants, for example, 25% less yield loss.

The above method may be used to select transgenic plants with increased yield, under water-limiting conditions and/or well-watered conditions, when compared to a control plant not comprising said recombinant DNA construct.

1. An isolated polynucleotide comprising:
(a) a nucleotide sequence encoding a polypeptide with GSH1 activity, wherein the polypeptide has an amino acid sequence of at least 97% sequence identity, based on the Clustal V method of alignment with pairwise alignment default parameters of KTUPLE=1, GAP PENALTY=3, WINDOW=5 and DIAGONALS SAVED=5, when compared to SEQ ID NO:2, 4, 6, 12, 14, 16, 18, 30, 32, 43 or 45; or
(b) the full complement of the nucleotide sequence of (a).

2. The polynucleotide of claim 1, wherein the amino acid sequence of the polypeptide comprises SEQ ID NO:2, 4, 6, 12, 14, 16, 18, 30, 32, 43 or 45.

3. The polynucleotide of claim 1 wherein the nucleotide sequence comprises SEQ ID NO:1, 3, 5, 11, 13, 15, 17, 29, 31, 42 or 44.

4. A recombinant DNA construct comprising the isolated polynucleotide of claim 1 operably linked to at least one regulatory sequence.

5. A plant or seed comprising the recombinant DNA construct of claim 4.

6. A plant comprising in its genome a recombinant DNA construct comprising a polynucleotide operably linked to at least one regulatory element, wherein said polynucleotide encodes a polypeptide having an amino acid sequence of at least 50% sequence identity, based on the Clustal V method of alignment, when compared to SEQ ID NO:2, 4, 6, 8, 10, 12, 14, 16, 18, 20, 22, 23, 24, 25, 26, 27, 28, 30, 32, 43, 45, 47, 48, 49 and 50, and wherein said plant exhibits an alteration of at least one agronomic characteristic when compared to a control plant not comprising said recombinant DNA construct.

7. The plant of claim 6, wherein said plant exhibits said alteration of said at least one agronomic characteristic when compared, under water limiting conditions, to said control plant not comprising said recombinant DNA construct.

8. The plant of claim 6, wherein said plant exhibits said alteration of said at least one agronomic characteristic when compared, under nitrogen limiting conditions, to said control plant not comprising said recombinant DNA construct.

9. The plant of claim 6, wherein said plant exhibits said alteration of said at least one agronomic characteristic when cultivated at a planting density higher than that which allows sufficient increases in biomass quantity per unit area and in seed yield per unit area for said control plant not comprising said recombinant DNA construct.

10. The plant of claim 6, wherein said at least one agronomic characteristic is at least one selected from the group consisting of greenness, yield, growth rate, biomass, fresh weight at maturation, dry weight at maturation, fruit yield, seed yield, total plant nitrogen content, fruit nitrogen content, seed nitrogen content, whole plant free amino acid content, fruit free amino acid content, seed free amino acid content, fruit protein content, seed protein content, protein content in a vegetative tissue, drought tolerance, nitrogen uptake, root lodging, harvest index, stalk lodging, plant height, ear height, ear length, early seedling vigor, seedling emergence under low temperature stress and disease resistance.

11. The plant of claim 6, wherein said plant exhibits an increase in seed yield, biomass, or both when compared to said control plant.

12. The plant of claim 6, wherein said plant further comprises and alteration in root architecture when compared to said control plant.

13. The plant of claim 6, wherein said plant is selected from the group consisting of: maize, soybean, sunflower, sorghum, canola, wheat, alfalfa, cotton, rice, barley, millet, sugar cane and switchgrass.

14. Seed of the plant of claim 6, wherein said seed comprises in its genome a recombinant DNA construct comprising a polynucleotide operably linked to at least one regulatory element, wherein said polynucleotide encodes a polypeptide having an amino acid sequence of at least 50% sequence identity, based on the Clustal V method of alignment, when compared to SEQ ID NO:2, 4, 6, 8, 10, 12, 14, 16, 18, 20, 22, 23, 24, 25, 26, 27, 28, 30, 32, 43, 45, 47, 48, 49 and 50, and wherein a plant produced from said seed exhibits an increase in at least one trait selected from the group consisting of: drought tolerance, seed yield and biomass, when compared to a control plant not comprising said recombinant DNA construct.

15. A method of determining an alteration of at least one agronomic characteristic in a plant, comprising:
(a) obtaining a transgenic plant, wherein the transgenic plant comprises in its genome a recombinant DNA construct comprising a polynucleotide operably linked to at least one regulatory element, wherein said polynucleotide encodes a polypeptide having an amino acid sequence of at least 50% sequence identity, based on the Clustal V method of alignment, when compared to SEQ ID NO:2, 4, 6, 8, 10, 12, 14, 16, 18, 20, 22, 23, 24, 25, 26, 27, 28, 30, 32, 43, 45, 47, 48, 49 and 50;
(b) obtaining a progeny plant derived from the transgenic plant, wherein the progeny plant comprises in its genome the recombinant DNA construct; and
(c) determining whether the transgenic plant exhibits an alteration of at least one agronomic characteristic when compared to a control plant not comprising the recombinant DNA construct.

16. The method of claim 15, wherein said determining step (c) comprises determining whether the transgenic plant exhibits an alteration of at least one agronomic characteristic when compared, under water limiting conditions, to a control plant not comprising the recombinant DNA construct.

17. The method of claim 15, wherein said determining step (c) comprises determining whether the transgenic plant exhibits an alteration of at least one agronomic characteristic when compared, under nitrogen limiting conditions, to a control plant not comprising the recombinant DNA construct.

18. The method of claim 15, wherein said determining step (c) comprises determining whether the transgenic plant exhibits an alteration of at least one agronomic characteristic when cultivated at a planting density higher than that which allows sufficient increases in biomass quantity per unit area and in seed yield per unit area for said control plant not comprising said recombinant DNA construct.

19. The method of claim 15, wherein said at least one agronomic characteristic is at least one selected from the group consisting of greenness, yield, growth rate, biomass, fresh weight at maturation, dry weight at maturation, fruit yield, seed yield, total plant nitrogen content, fruit nitrogen content, seed nitrogen content, whole plant free amino acid content, fruit free amino acid content, seed free amino acid content, fruit protein content, seed protein content, protein content in a vegetative tissue, drought tolerance, nitrogen uptake, root lodging, harvest index, stalk lodging, plant height, ear height, ear length, early seedling vigor, seedling emergence under low temperature stress and disease resistance.

20. The method of claim 15, wherein said plant exhibits an increase in seed yield, biomass, or both when compared to said control plant.

21. The method of claim 15, wherein said plant further comprises and alteration in root architecture when compared to said control plant.

22. The method of claim 15, wherein said plant is selected from the group consisting of: maize, soybean, sunflower, sorghum, canola, wheat, alfalfa, cotton, rice, barley, millet, sugar cane and switchgrass.
